(19) United States
(12) Patent Application Publication
(10) Pub. No.: US 2013/0065857 A1
(43) Pub. Date: Mar. 14, 2013

(54) NOVEL DXR INHIBITORS FOR ANTIMICROBIAL THERAPY

(71) Applicant: Yongcheng Song, Pearland, TX (US)

(72) Inventor: Yongcheng Song, Pearland, TX (US)

(73) Assignee: Baylor College of Medicine, Houston, TX (US)

(21) Appl. No.: 13/501,636

(22) PCT Filed: Oct. 12, 2010
(86) PCT No.: PCT/US10/52296

§ 371 Date: Nov. 29, 2012

Related U.S. Application Data
(60) Provisional application No. 61/250,747, filed on Oct. 12, 2009.

(51) Int. Cl. A61K 31/675; A61P 31/00; A61K 31/683; C07F 9/58; C07F 9/60

(52) U.S. Cl. 514/82; 546/22; 514/89

(57) ABSTRACT

The present invention generally concerns particular methods and compositions for antimicrobial therapy. In particular embodiments, the compositions target DXR. In specific embodiments, the compositions are electron-deficient heterocyclic rings.

### CROSS-REFERENCE TO RELATED APPLICATIONS

The present invention claims priority to U.S. Provisional Patent Application Ser. No. 61/250,747, filed Oct. 12, 2009, which is incorporated by reference herein in its entirety.

### STATEMENT REGARDING FEDERALLY SPONSORED RESEARCH OR DEVELOPMENT

This invention was made with government support under R21AI088123 awarded by National Institute for Allergy and Infectious Diseases (NIAID). The government has certain rights in the invention.

### TECHNICAL FIELD

The present invention generally concerns at least the fields of cell biology, molecular biology, pathology, and medicine. In particular cases, the present invention concerns antimicrobial compositions and methods related thereto.

### BACKGROUND OF THE INVENTION

The development of antibiotics is considered the most successful story in drug discovery, having saved millions of people's lives since the widespread prescription of penicillin in 1940s. However, according to the World Health Organization (WHO), bacterial infections are still the number one cause of human death, killing ˜6 million people each year worldwide, mostly in developing countries. In addition, drug resistant bacteria have reached epidemic levels during the past few decades. For example, tuberculosis alone causes the deaths of ˜1.6 million people annually, with Mycobacterium tuberculosis, the causative agent, becoming more and more drug resistant: in many countries with a high incidence of tuberculosis (e.g., China), ˜20% cases of newly diagnosed tuberculosis are now resistant to the most widely used drug isoniazid, while the number increases to >45% among previously treated patients.

Even in the United States, bacterial infections have also become a serious threat and burden to public health mainly because of the rising drug resistance. For instance, P. aeruginosa infections account for ˜10 percent of hospital-acquired infections. This Gram-negative bacterium is notorious for its inherited resistance to antibiotics and is therefore a particularly dangerous and dreaded pathogen (Driscoll et al., 2007; Paterson, 2006). Only a few antibiotics are effective, including gentamicin, imipenem, and fluoroquinolones, and even these antibiotics are not effective against all strains. New strains resistant to these antibiotics have continued to emerge. For example, many strains of P. aeruginosa have now acquired metallo-β-lactamase genes and therefore become highly resistant to imipenem (Walsh, 2005; Walsh et al., 2005). Few options are available to treat infections caused by this multiple drug resistant bacterium. Pseudomonas infections are thus a life-threatening disease for patients with cystic fibrosis and severe burns, as well as cancer and AIDS patients who are immuno-compromised.

On the other hand, production of new antibacterial drugs by the pharmaceutical industry has decreased significantly since 1980 (Nathan, 2004). The reasons are complex but may be due mainly to a poor investment yield on anti-infective drugs (Christoffersen, 2006). There is, therefore, an urgent need to find new drugs to combat bacterial infections that are resistant to the current therapies (Nat. Rev. Drug Discov., 2007). In addition, one important strategy to overcome the rising drug resistance is to use combination therapy to treat bacterial infections (Walsh, 2003). The combination of two or more drugs without cross-resistance, which act on different targets, will significantly reduce the likelihood of resistance. However, unfortunately, the common antibiotics such as methicillin and vancomycin have not been used in combination therapy.

Another serious infectious disease is malaria, the so-called “most neglected disease”. Around 2.5 billion people or 40% of the world's population live at risk of malaria, which afflicts about 300-500 million people and kills ˜1.5 million per year. These dreadful numbers will be likely rising mainly because of the increased drug resistance of malaria parasites against commonly used, cheap drugs like chloroquine. In addition, because of the extreme poverty in affected areas, pharmaceutical industry has had little involvement in antimalarial drug discovery/development (Pecoul et al., 1999; Trouiller et al., 2002). For example, during the period 1975-1997 there were 1223 new chemical entities (NCEs) commercialized, of which only 4 (0.3%) are specifically for the treatment of malaria.

DXR is the 2nd enzyme in the non-mevalonate isoprene biosynthesis pathway, as shown in FIG. 1A (Hunter, 2007). This is used by most pathogenic bacteria (except Gram-positive cocci), such as M. tuberculosis, as well as malaria parasites, to make essential isopentenyl diphosphate (IPP) and dimethylallyl diphosphate (DMAPP), which are two common precursors for biosynthesis of all isoprenoids/terpenoids. DXR is essential for the growth of these species. On the other hand, humans and animals use the mevalonate pathway (FIG. 1A) to make IPP and DMAPP, making DXR an attractive drug target for novel anti-infectives. Although considerable progress has been achieved in understanding its biochemical and structural properties during the past decade, fosmidomycin, a naturally occurring antibiotic found in 1980 (Mine et al., 1980; Neu and Kamimura, 1981), together with its close analogs such as FR900098 (FIG. 1B), have been the only potent inhibitors of DXR (Hunter, 2007; Kuzuyama et al., 1998). Indeed, as a potent inhibitor (IC₅₀: 28 nM) of DXR from P. falciparum (Jomaa et al., 1999), fosmidomycin has potent anti-malarial activity, rapidly clearing the parasites from patients' blood (Missinou et al., 2002; Borrmann et al., 2004; Borrmann et al., 2006; Borrmann et al., 2005; Oyakhirome et al., 2007). However, it has a relatively poor pharmacokinetic profile, quickly eliminated from patients' body with a half live in plasma ranging from 0.5-1.5 h. High doses, i.e., 3.6 g/day for two weeks, are therefore required to achieve the desired pharmacological effects.

In addition, fosmidomycin is also a potent inhibitor of DXRs from bacterial species (Kuzuyama et al., 1998; Altincicek et al., 2000; dhiman et al., 2005) and has activity against most gram-negative bacteria such as P. aeruginosa (Mine et al., 1980; Neu and Kamimura, 1981). For example, it inhibits 50% of P. aeruginosa isolates at the minimal inhibition concentration (MIC₅₀) of 6.25 μg/mL, more active than clinically used gentamicin (MIC50: 12.5 μg/mL) (Neu and Kamimura, 1981). It has also excellent activity against many gram-negative bacteria such as E. coli, H. influenzae and Enterobacter sp. However, many strains of these bacteria are now resistant to fosmidomycin. Furthermore, Gram-positive bacteria, such as M. tuberculosis and B. cereus, are generally not sensitive to fosmidomycin. This is attributed to that fosmidomycin, a highly polar and non-lipophilic molecule, is excluded from resistant bacterial cells. Fosmidomycin is transported into the sensitive bacteria and parasites via a glycerol 3-phosphate transporter GlpT. Bacteria lacking GlpT or with a mutant/unfunctional glpT protein are therefore resistant to fosmidomycin by either limited uptake or effective efflux (Dhiman et al., 2005; Brown and Parish, 2008; sakamoto et al., 2003). Nonetheless, fosmidomycin remains a strong inhibitor of M. tuberculosis DXR with IC₅₀ of 310 nM (Dhiman et al., 2005). Thus, DXR is still a valid target for anti-bacterial drug discovery, so there is a need to find potent inhibitors of bacterial DXRs with good permeability into bacterial cells. These compounds would be a useful, novel class of antibiotics without cross drug resistance.

Another piece of evidence that indicates the need of a new type of DXR inhibitors comes from recent work with T. gondii, which is the causative agent of toxoplasmosis. This protozoan parasite infects most warm-blooded animals, including cats (the primary host) as well as humans. The infection is generally mild for healthy people but can have serious or even fatal effects on a fetus whose mother carries the parasite during pregnancy or on an immuno-compromised person (e.g., HIV, cancer and organ transplant patients). T. gondii is estimated to infect up to ⅓ of world population (Montoya and Liesenfeld, 2004) and the CDC reported that the prevalence of this disease in the US is 11%, including women of childbearing age who are particularly at risk (Jones et al., 2007). Recent study from the Moreno group showed that DXR is essential for the growth of T. gondii, but fosmidomycin has no activity on the parasite. This shows that either it cannot enter into the parasite cells or it is a poor inhibitor of T. gondii DXR. However, in any case, new DXR inhibitors are needed.

DXR catalyzes the isomerization and reduction of 1-deoxy-D-xylulose-5-phosphate (DXP) to 2-C-methyl-D-erythritol 4-phosphate (MEP) in the presence of Mg²⁺ and NADPH, which is a hydride donor (Takahashi et al., 1998), as shown in Scheme 1.

The structure and function of DXR have been actively studied during the past decade and about a dozen of x-ray structures of DXRs from several species (e.g., E. coli and M. tuberculosis), complexed with various combinations of the substrate, inhibitors and cofactors, have been published (Henriksson et al., 2007; Mac Sweeney et al., 2005; Ricagno et al., 2004; Yajima et al., 2007; Yajima et al., 2002; Yajima et al., 2002). The representative quaternary DXR crystal structure (Yajima et al., 2007) in complex with fosmidomycin, Mg²⁺ and NADPH, is shown in FIG. 2. The Mg²⁺ is coordinated in a distorted octahedral configuration with the two oxygen atoms of hydroxamate, Glu 152 and 231, Asp 150, and a water molecule. The substrate DXP binds to the enzyme at the same site as fosmidomycin, shown superimposed in FIG. 2. The phosph(on)ate group has H-bond and electrostatic interactions with the Lys228 and Ser185 residues. The nicotinamide ring of NADPH is located in a mainly hydrophobic pocket with an orientation that would allow the transfer of a C4 hydride to the substrate.

As a promising anti-infective drug target, much interest has been attracted to develop DXR inhibitors during the past decade (Shtannikov et al., 2007; Yajima et al., 2004; Gottlin et al., 2003; Kuntz et al., 2005; Merckle et al., 2005; Munos et al., 2008; Ortmann et al., 2007; Silber et al., 2005; Woo et al., 2006). Despite these efforts using either high-throughput screening or medicinal chemistry based on the structures of fosmidomycin/DXP, no other potent DXR inhibitors (IC₅₀s <1 μM) have been identified. This reflects the challenge in discovering potent DXR inhibitors. For example, a high-throughput screening of 32,000 compounds only yielded 30 hits with IC₅₀s of <20 μM (Gottlin et al., 2003). However, the structures of these hits were not disclosed and these compounds therefore cannot be confirmed and further developed.

There is a need in the art to provide additional anti-pathogenic compounds for the treatment of infections, including DXR inhibitors that are useful for antimicrobial therapy.

### BRIEF SUMMARY OF THE INVENTION

The present invention generally concerns methods and compositions for antimicrobial therapy. The antimicrobial therapy may be effective against any kind of microbe, but in specific embodiments the microbe is a bacterium, fungus, protozoan or virus, for example. In specific embodiments, the microbe is a bacterium. In particular cases, the microbe has the enzyme 1-Deoxy-D-xylulose-5-phosphate reductoisomerase (DXR) and the antimicrobial composition targets DXR, although in alternative embodiments the microbe lacks DXR but the composition is still effective against the microbe. In some cases, the antimicrobial composition is effective against one or more microbes that are resistant to one or more other antimicrobial therapies. In some cases, the antimicrobial agent comprises an electron-deficient hydrophobic group that has interacts with Trp211 of DXR. In specific embodiments, the compound contains electron-deficient heterocyclic rings that specifically interact with the electron-rich indole ring of Trp211, for example.

Exemplary microbes that have DXR include but are not limited to Mycobacterium tuberculosis, Helicobacter pylori, Listeria monozytogenes, Escherichia coli, Pseudomonas aeruginosa, Haemophilus influenzae, Bacillus cereus, and Bacillus subtilis.

In specific embodiments, the antimicrobial agent is effective against one or more bacteria selected from the group consisting of the following phyla: 1) Aquificae; 2) Xenobacteria; 3) Fibrobacter; 4) Bacteroids; 5) Firmicutes; 6) Planctomycetes; 7) Chrysogenetic; 8) Cyanobacteria; 9) Thermomicrobia; 10) Chlorobia; 11) Proteobacteria; 12) Spirochaetes; 13) Flavobacteria; 14) Fusobacteria; and 15) Verrucomicrobia. In specific cases, the disinfectants of the present invention are useful against Gram positive cocci; Gram negative cocci; Gram positive bacilli; Gram negative bacilli, Spirochaetes, Rickettsia, and Mycoplasma.

In certain cases, the disinfectants are useful against Staphylococcus, Streptococcus, Corynebacterium, Listeria, Bacillus, Clostridium, Neisseria, Enterobacteria, E. coli, Salmonella, Shigella, Campylobacter, Chlamydia, Borrelia, Francisella, Leptospira, Treponema, Proteus, Yersinia pestis, Vibrio, Helicobacter, Haemophila, Bordetella, Brucella, and Bacteriodes. In particular cases, the disinfectants are useful against Staphylococcus aureus, Listeria monocytogenes, Clostridium botulinum, Legionella pneumophila, E. coli, Salmonella enterica, Neisseria meningitides, Yersinia pestis, Mycobacterium tuberculosis, Vibrio cholera, Group A hemolytic streptococci, Diplococcus pneumonia, Moraxella catarrhalis, Neisseria gonorrhoeae, C. jeikeium, Mycobacterium avium complex, M. kansasii, M. leprae, M. tuberculosis, Nocardia sp, Acinetobacter calcoaceticus, Flavobacterium meningosepticum, Pseudomonas aeruginosa, P. alcaligenes, other Pseudomonas sp, Stenotrophomonas maltophilia, Brucella, Bordetella, Francisella, Legionella spp, Leptospira sp, Bacteroides fragilis, other Bacteroides sp, Fusobacterium sp, Prevotella sp, Veillonella sp, Peptococcus niger, Peptostreptococcus sp, Actinomyces, Bifidobacterium, Eubacterium, and Propionibacterium spp, Clostridium botulinum, C. perfringens, C. tetani, other Clostridium sp, Staphylococcus aureus (coagulase-positive), S. epidermidis (coagulase-negative), other coagulase-negative staphylococci, Enterococcus faecalis, E. faecium, Streptococcus agalactiae (group B streptococcus), S. Bovis, S. pneumoniae, S. pyogenes (group A streptococcus), viridans group streptococci (S. mutans, S. mitis, S. salivarius, S. sanguis), S. anginosus group (S. anginosus, S. milleri, S. constellatus), Gemella morbillorum. Bacillus anthracis, Erysipelothrix rhusiopathiae, Gardnerella vaginalis (gram-variable), Enterobacteriaceae (Citrobacter sp, Enterobacter aerogenes, Escherichia coli, Klebsiella sp, Morganella morganii, Proteus sp, Providencia rettgeri, Salmonella typhi, other Salmonella sp, Serratia marcescens, Shigella sp, Yersinia enterocolitica, Y. pestis), Aeromonas hydrophila, Chromobacterium violaceum, Pasturella multocida, Plesiomonas shigelloides, Actinobacillus actinomycetemcomitans, Bartonella bacilliformis, B. henselae, B. quintana, Eikenella corrodens, Haemophilus influenzae, other Haemophilus sp, Mycoplasma pneumonia, Borrelia burgdorferi, Treponema pallidum Campylobacter jejuni, Helicobacter pylori, Vibrio cholerae, V. vulnificus, Chlamydia trachomatis, Chlamydophila pneumoniae, C. psittaci, Coxiella burnetii, Rickettsia prowazekii, R. rickettsii, R. typhi, R. tsutsugamushi, R. africae, R. akari, Ehrlichia canis, Ehrlichia chaffeensis, and Anaplasma phagocytophilum.

In particular embodiments of the present invention, the antimicrobial agent is effective against one or more viruses, including one or more pathogenic viruses. In specific embodiments, the antimicrobial agent is effective against one or more viruses selected from the group consisting of Adenoviridae, Picornaviridae, Herpesviridae, Hepadnaviridae, Flaviviridae, Retroviridae, Orthomyxoviridae, Parvoviridae, Paramyxoviridae, Papovaviridae, Polyomavirus, Rhabdoviridae, and Togaviridae. Particular viruses include, for example, HIV, Adenovirus Influenza A, Rabies virus, Hepadnavirus, Varicella-zoster virus, Herpes simplex virus (types 1 and 2), Ebolavirus, Epstein Barr virus, Varicella-zoster virus, pox virus (including smallpox, copox, or monkey pox), human cytomegalovirus, poliovirus, coxsackievirus, Rubeola virus (paramyxovirus), Rubella virus, Variola virus, Avian flu virus (Influenza A virus), hepatitis A, B, and C viruses, parainfluenza, mumps virus, measles virus, respiratory syncitial virus, West Nile virus, Dengue fever virus, yellow fever virus, foot and mouth disease virus, and severe acute respiratory syndrome (SARS) coronavirus.

In particular embodiments of the present invention, the antimicrobial agent is effective against one or more fungi, including one or more pathogenic fungi. In specific embodiments, the antimicrobial agent is effective against one or more fungi selected from the group consisting of Histoplasma, Aspergillus and other common household molds, Candida, Cryptococcus, Stachybotrys, Zygomycosis, Fusarium, Blastomycosis, Coccidioides, Scedosporium, and Pneumocystis.

In some cases, the antimicrobial composition is employed against prions.

In certain embodiments of the invention, the antimicrobial therapy comprises one or more compositions encompassed by the invention. The antimicrobial composition may be formulated in a pharmaceutical composition. In specific embodiments, the composition is administered to an individual that has an infection of the microbe, has been exposed to the microbe, or that may be exposed to the microbe. In certain embodiments, the antimicrobial therapy of the invention is given to an individual that will receive, is receiving, or has received another therapy for the microbe. In specific cases, the effective composition is preventative of infection of the microbe.

In particular cases, the antimicrobial composition is delivered to a mammal, including a human, dog, cat, horse, goat, sheep, cow, or pig. In specific embodiments, the antimicrobial composition is delivered systemically or non-systemically. The composition may be delivered by injection, topically, or orally, for example. The composition may be delivered to the individual in a single dose or in multiple doses. Multiples doses may be delivered over the course of a single day, over the course of two or more days, one week, two weeks, or more.

Embodiments of the present invention include methods of producing the compositions of the invention and methods of treating and/or preventing infection in an individual.

Other and further objects, features, and advantages would be apparent and eventually more readily understood by reading the following specification and be reference to the accompanying drawings forming a part thereof, or any examples of the presently preferred embodiments of the invention given for the purpose of the disclosure.

### BRIEF DESCRIPTION OF THE DRAWINGS

FIG. 1A illustrates the non-mevalonate and the mevalonate isoprene pathways, together with inhibitors. FIG. 1B shows the structures of fosmidomycin and FR900098.

FIG. 2 shows crystal structure of DXR-fosmidomycin-Mg²⁺-NADPH complex, with superimposed DXP (in orange). Mg²⁺ is shown as a pink sphere.

FIG. 3 demonstrates molecular surface (50% transparency) of DXR, showing a large hydrophobic pocket occupied by the side chain of bisphosphonate inhibitor 1 (in orange). Together superimposed are fosmidomycin (with C atoms in green) and NADPH (with C atoms in blue).

FIG. 4 shows exemplary DXR inhibitors.

FIG. 5 provides dose response curves of compounds 3 and 8 of FIG. 4.

FIG. 6 provides a ClustalX alignment of DXR from E. coli (Ec), P. aeruginosa (Pa), M. tuberculosis (Mt) and P. falciparum (Pf), showing 23% identity as well as 52% similarity.

FIG. 7 illustrates (A) The active site of DXR with 20 docking structures of compound 8, which forms a tight cluster. The crystal structure of fosmidomycin (in yellow) is also superimposed. (B) The docking structure of 8 with the lowest docking score is shown.

FIG. 8 provides an exemplary plan for medicinal chemistry modifications of exemplary compound 8.

FIG. 9 shows that the tenofovir prodrug tenofovir disoproxil (Viread) is hydrolyzed in vivo or in cells by an esterase to give tenofovir. The esterase cleavage sites are marked with red arrows.

FIG. 10 illustrates the active site of crystal structure of the DXR-1 complex, superimposed with the crystal structure of DXP (carbon atoms in grey). Mg²⁺ is shown as a pink sphere.

FIG. 11 illustrates A) A docking structure of 4 (ball and stick model) in DXR, with superimposed crystal structure of 1. B) A docking structure of 6 in DXR, showing its phenyl group in a hydrophobic pocket. Mg²⁺ is shown as a pink sphere.

FIG. 12 shows the overall structures of (A) DXR:17, (B) DXR:18 and (C) DXR:19 complexes. The compounds bound to DXR were shown in stick models with Fo-Fc omit maps calculated by the program CNS. The maps were contoured at 3σ for 17 and 1.5 σ for Trp211 (D), 2.5 σ for 18 and 1 σ for Trp211 (E) and 3 σ for 19 and 2.5 σ for Trp211 (F), respectively.

FIG. 13 provides (A) superimposed structures of 17-19 and 1 (in cyan) in DXR with NADPH; (B) The active site of the DXR:17 complex; (C) The active site of the DXR:18 complex; (D) The active site of the DXR:19 complex.

FIG. 14 shows superposition of the DXR:17 (with green carbon atoms) and DXR:1 (in yellow) structures, showing sidechains of Asp149, Glu151 and Glu230 in the DXR:17 complex do not deviate considerably without a bound Mg²⁺. Mg²⁺ is shown as a pink sphere.

FIG. 15 shows (A) protein backbones of the superimposed structures of the DXR:17-19 complexes; (B) Tube models of the superimposed structures of the DXR:18 (in green), DXR:1 (in gray) and DXR:9 (in orange) complexes, showing major conformational changes for the flexible loop in the active site; (C) Close-up view of the superimposed active sites of the DXR:18 (in green), DXR:1 (in gray) and DXR:9 (in orange) complexes, showing the indole ring of Trp211 moves considerably to recognize different inhibitors bound to DXR. Mg2+ is shown as a pink sphere.

FIG. 16 demonstrates (A) 10 docking structures of 1 using the DXR:1 structure, superimposed with the crystal structure of 1 (in yellow), showing rms deviations of 0.65 to 1.5 Å. Mg²⁺ is shown as a pink sphere. (B) 10 docking structures of 10 using the DXR:10 structure, superimposed with the crystal structure of 10 (in yellow), showing rms deviations of 1.1 to 1.9 Å. (C) 10 docking structures of 17 using the DXR:17 structure, superimposed with the crystal structure of 17 (in yellow), showing rms deviations of 0.37 to 1.5 Å.

FIG. 17 illustrates (A) Docking result of (R)-8 using the DXR:17 structure with a Mg²⁺ (pink sphere); (B) The lowest energy docking structure of (R)-8, superimposed with 1 (in yellow), showing (R)-8 is predicted to bind favorably to DXR; (C) Docking result of (S)-8 using the DXR:17 structure, exhibiting a “reversed” binding mode; (D) Docking result of (R)-8 using the DXR:1 structure; (E) Docking result of (R)-8 using the DXR:10 structure; (F) The lowest-energy docking structure of (R)-3 using the DXR:17 structure, superimposed with the crystal structures of 1 (in yellow) and 17 (in green).

FIG. 18 illustrates (A) Docking result of (S)-8 using the DXR:1 structure; (B) Docking result of (S)-8 using the DXR:10 structure. All show a “reversed” binding mode.

FIG. 19 shows docking structures (ball and stick model) of compounds 4-7 (A-D, respectively) with the lowest energy using the DXR:17 structure. The crystal structures of 1 (in yellow) and 17 (in green) are superimposed. Mg²⁺ is shown as a pink sphere.

FIGS. 20 and 21 illustrate exemplary compositions of the invention.

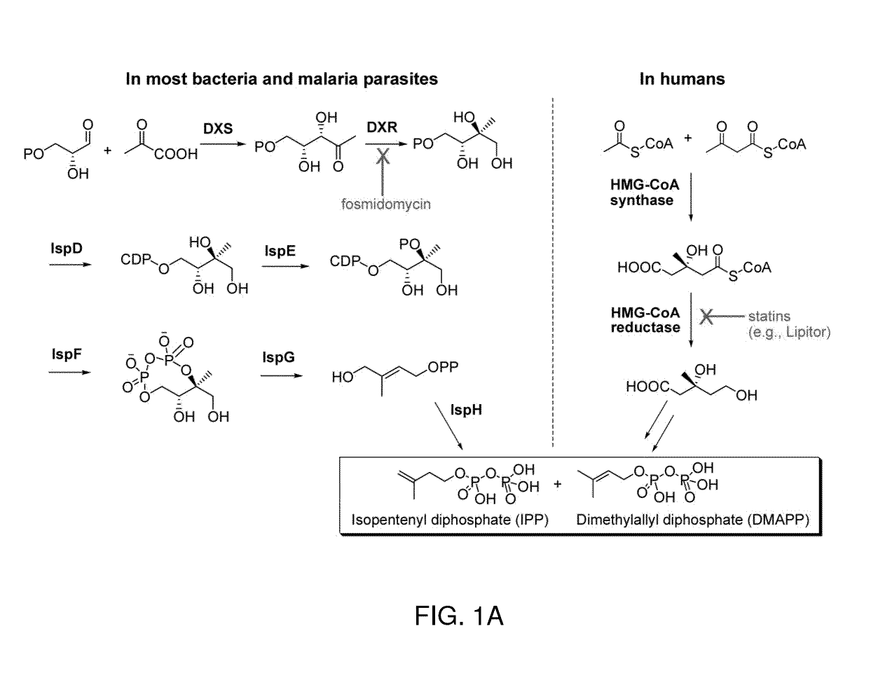

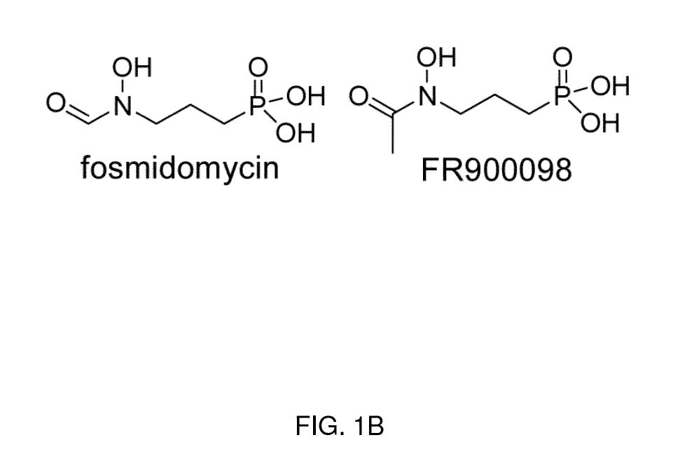

### DETAILED DESCRIPTION OF THE INVENTION

As used herein, the use of the word “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method or composition described herein can be implemented with respect to any other method or composition described herein.

The present invention has utilized a combination of traditional medicinal chemistry and computational, structure based drug design to develop novel small molecule inhibitors of 1-deoxy-D-xylulose-5-phosphate reductoisomerase (DXR) whose activity may be tested in vitro on pathogenic bacteria and parasites. DXR is a validated target for anti-infective drug discovery. The present invention provides novel inhibitors that are clinically useful anti-infective drugs to treat bacterial infections, malaria and other parasitic diseases, caused by, e.g., Pseudomonas aeruginosa, Mycobacterium tuberculosis, Plasmodium falciparum and Toxoplasma gondii.

Isoprene biosynthesis is essential to all organisms. Humans and animals use the mevalonate pathway to produce isopentenyl diphosphate (IPP) and dimethylallyl diphosphate (DMAPP), two common precursors for all isoprenoid biosynthesis; however, in most pathogenic bacteria, such as P. aeruginosa and M. tuberculosis, as well as apicomplexan parasites, such as P. falciparum and T. gondii, the non-mevalonate pathway, or 2C-methyl-D-erythritol-4-phosphate (MEP) pathway, is used to make IPP and DMAPP (Hunter, 2007). Since humans lack all the 7 enzymes in the non-mevalonate pathway, it has become an attractive target for anti-infective drug discovery (Rodriguez-Concepcion, 2004; Singh et al., 2007; Testa and Brown, 2003). Fosmidomycin has been found to be the only potent inhibitor of this pathway, blocking DXR, the 2nd enzyme, and has antibacterial activity against many Gram-negative bacteria (Mine et al., 1980; Neu and Kamimura, 1981) and antimalarial activity in recent clinical trials (Missinou et al., 2002; Borrmann et al., 2004; Borrmann et al., 2006; Borrmann et al., 2005; Oyakhirome et al., 2007). However, Gram-positive bacteria (e.g., M. tuberculosis) and some Gram-negative bacteria (Shtannikov et al., 2007) as well as certain pathogenic parasites (e.g., T. gondii) are resistant to fosmidomycin. In addition, it has a poor pharmacokinetic profile with a half-life in plasma of 0.5-1.5 h. Given the current devastating situation facing quickly rising drug resistance as well as shortage of new anti-infective drugs, there is a pressing need to find new weaponry for infectious diseases. Based on rational, structure based design, the present invention provides a submicromolar inhibitor of DXR with a distinct structure from that of fosmidomycin.

In specific aspects, a combination of traditional medicinal chemistry and computational, structure based drug design is used to develop novel inhibitors of 1-deoxy-D-xylulose-5-phosphate reductoisomerase (DXR). Since fosmidomycin is the only potent DXR inhibitor but, due to its very polar structure and poor pharmacokinetic properties, it has no activity against many bacteria and pathogenic parasites, novel, more lipophilic DXR inhibitors are now needed. Based on rational, structure based design, the inventors have found novel, drug-like lead inhibitors with Kᵢs as low as 310 nM against a recombinant E. coli DXR enzyme. The docking studies showed that they could bind to DXR in a different mode from that of fosmidomycin. In development of the present invention, there is 1) use of medicinal chemistry to make several series of compound libraries based on the scaffold of the lead inhibitor, to find compounds with improved activity; 2) carrying out of quantitative structure activity relationship (QSAR) studies of these compounds; 3) obtaining of x-ray crystal structures of DXR in complex with novel inhibitors; and 4) use of the results from the computational and crystallographic studies to characterize further drug design and synthesis.

One can test in vitro biological activities of lead inhibitors as well as potent inhibitors. In some cases, a recombinant E. coli DXR is used as a primary screen. Good inhibitors against the E. coli enzyme are further tested against DXRs from M. tuberculosis, P. falciparum and T. gondii, for example, in order to obtain an inhibition/selectivity profile of novel DXR inhibitors. Next, one can test the activities of these DXR inhibitors on a broad range of bacteria as well as apicomplexan parasites, including E. coli, P. aeruginosa, Haemophilus influenzae, Bacillus subtilis, Bacillus cereus, M. tuberculosis, P. falciparum and T. gondii, for example These species include 3 Gram-negative, 3 Gram-positive bacteria and 2 eukaryotic parasites, with several being notorious pathogens that are responsible for deaths of millions of people each year. Finally, one can also test the cytotoxicity of potent DXR inhibitors on mammalian cell lines (e.g., 3T3) to evaluate their potential toxicity.

### I. Exemplary Chemical Group Definitions

When used in the context of a chemical group, “hydrogen” means —H; “hydroxy” means —OH; “oxo” means ═o; “halo” means independently —F, —Cl, —Br or —I; “amino” means —NH₂ (see below for definitions of groups containing the term amino, e.g., alkylamino); “hydroxyamino” means —NHOH; “nitro” means —NO₂; imino means ═NH (see below for definitions of groups containing the term imino, e.g., alkylimino); “cyano” means —CN; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; “thio” means ═S; “thioether” means —S—; “sulfonamido” means —NHS(O)₂—(see below for definitions of groups containing the term sulfonamido, e.g., alkylsulfonamido); “sulfonyl” means —S(O)₂— (see below for definitions of groups containing the term sulfonyl, e.g., alkylsulfonyl); “sulfinyl” means —S(O)— (see below for definitions of groups containing the term sulfinyl, e.g., alkylsulfinyl); and “silyl” means —SiH₃ (see below for definitions of group(s) containing the term silyl, e.g., alkylsilyl).

The symbol “—” means a single bond, “═” means a double bond, and “≡” means triple bond. The symbol “ [symbol] ” represents a single bond or a double bond. The symbol “ [symbol] ”, when drawn perpendicularly across a bond indicates a point of attachment of the group. It is noted that the point of attachment is typically only identified in this manner for larger groups in order to assist the reader in rapidly and unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the conformation is unknown (e.g., either R or S), the geometry is unknown (e.g., either E or Z) or the compound is present as mixture of conformation or geometries (e.g., a 50%/50% mixture).

When a group “R” is depicted as a “floating group” on a ring system, for example, in the formula:

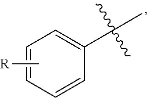

then R may replace any hydrogen atom attached to any of the ring atoms, including a depicted, implied, or expressly defined hydrogen, so long as a stable structure is formed.

When a group “R” is depicted as a “floating group” on a fused ring system, as for example in the formula:

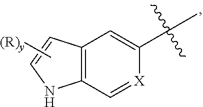

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

When y is 2 and “(R)_{y}” is depicted as a floating group on a ring system having one or more ring atoms having two replaceable hydrogens, e.g., a saturated ring carbon, as for example in the formula:

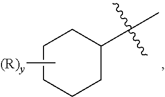

then each of the two R groups can reside on the same or a different ring atom. For example, when R is methyl and both R groups are attached to the same ring atom, a geminal dimethyl group results. Where specifically provided for, two R groups may be taken together to form a divalent group, such as one of the divalent groups further defined below. When such a divalent group is attached to the same ring atom, a spirocyclic ring structure will result.

When the point of attachment is depicted as “floating”, for example, in the formula:

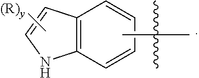

then the point of attachment may replace any replaceable hydrogen atom on any of the ring atoms of either of the fused rings unless specified otherwise.

In the case of a double-bonded R group (e.g., oxo, imino, thio, alkylidene, etc.), any pair of implicit or explicit hydrogen atoms attached to one ring atom can be replaced by the R group. This concept is exemplified below:

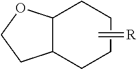

represents

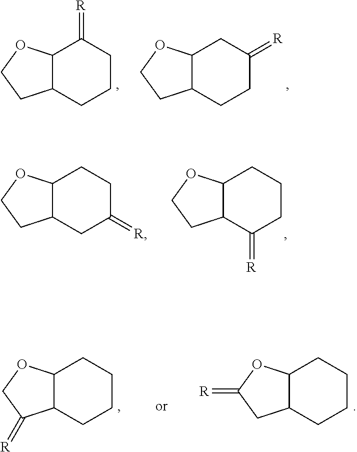

For the groups and classes below, the following parenthetical subscripts further define the group/class as follows: “(Cn)” defines the exact number (n) of carbon atoms in the group/class. “(C≦n)” defines the maximum number (n) of carbon atoms that can be in the group/class, with the minimum number as small as possible for the group in question, e.g., it is understood that the minimum number of carbon atoms in the group “alkenyl_{(C≦8)}” or the class “alkene_{(C≦8)}” is two. For example, “alkoxy_{(C≦10)}” designates those alkoxy groups having from 1 to 10 carbon atoms (e.g., 1, 2, 3, 4, 5, 6, 7, 8, 9, or 10, or any range derivable therein (e.g., 3 to 10 carbon atoms). (Cn-n′) defines both the minimum (n) and maximum number (n′) of carbon atoms in the group. Similarly, “alkyl_{(C2-10)}” designates those alkyl groups having from 2 to 10 carbon atoms (e.g., 2, 3, 4, 5, 6, 7, 8, 9, or 10, or any range derivable therein (e.g., 3 to 10 carbon atoms)).

The term “alkyl” when used without the “substituted” modifier refers to a non-aromatic monovalent group with a saturated carbon atom as the point of attachment, a linear or branched, cyclo, cyclic or acyclic structure, no carbon-carbon double or triple bonds, and no atoms other than carbon and hydrogen. The groups, —CH₃ (Me), —CH₂CH₃ (Et), —CH₂CH₂CH₃ (n-Pr), —CH(CH₃)₂ (iso-Pr), —CH(CH₂)₂ (cyclopropyl), —CH₂CH₂CH₂CH₃ (n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), cyclobutyl, cyclopentyl, cyclohexyl, and cyclohexylmethyl are non-limiting examples of alkyl groups. The term “substituted alkyl” refers to a non-aromatic monovalent group with a saturated carbon atom as the point of attachment, a linear or branched, cyclo, cyclic or acyclic structure, no carbon-carbon double or triple bonds, and at least one atom independently selected from the group consisting of N, O, F, Cl, Br, I, Si, P, and S. The following groups are non-limiting examples of substituted alkyl groups: —CH₂OH, —CH₂Cl, —CH₂Br, —CH₂SH, —CF₃, —CH₂CN, —CH₂C(O)H, —CH₂C(O)OH, —CH₂C(O)OCH₃, —CH₂C(O)NH₂, —CH₂C(O)NHCH₃, —CH₂C(O)CH₃, —CH₂OCH₃, —CH₂OCH₂CF₃, —CH₂OC(O)CH₃, —CH₂NH₂, —CH₂NHCH₃, —CH₂N(CH₃)₂, —CH₂CH₂Cl, —CH₂CH₂OH, —CH₂CF₃, —CH₂CH₂OC(O)CH₃, —CH₂CH₂NHCO₂C(CH₃)₃, and —CH₂Si(CH₃)₃.

The term “alkanediyl” when used without the “substituted” modifier refers to a non-aromatic divalent group, wherein the alkanediyl group is attached with two σ-bonds, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, —CH₂CH₂CH₂—, and

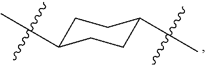 [SMILES: *C1CCC(*)CC1]

are non-limiting examples of alkanediyl groups. The term “substituted alkanediyl” refers to a non-aromatic monovalent group, wherein the alkynediyl group is attached with two σ-bonds, with one or two saturated carbon atom(s) as the point(s) of attachment, a linear or branched, cyclo, cyclic or acyclic structure, no carbon-carbon double or triple bonds, and at least one atom independently selected from the group consisting of N, O, F, Cl, Br, I, Si, P, and S. The following groups are non-limiting examples of substituted alkanediyl groups: —CH(F)—, —CF₂—, —CH(Cl)—, —CH(OH)—, —CH(OCH₃)—, and —CH₂CH(Cl)—.

The term “alkane” when used without the “substituted” modifier refers to a non-aromatic hydrocarbon consisting only of saturated carbon atoms and hydrogen and having a linear or branched, cyclo, cyclic or acyclic structure. Thus, as used herein cycloalkane is a subset of alkane. The compounds CH₄ (methane), CH₃CH₃ (ethane), CH₃CH₂CH₃ (propane), (CH₂)₃ (cyclopropane), CH₃CH₂CH₂CH₃ (n-butane), and CH₃CH(CH₃)CH₃ (isobutane), are non-limiting examples of alkanes. A “substituted alkane” differs from an alkane in that it also comprises at least one atom independently selected from the group consisting of N, O, F, Cl, Br, I, Si, P, and S. The following compounds are non-limiting examples of substituted alkanes: CH₃OH, CH₃Cl, nitromethane, CF₄, CH₃OCH₃ and CH₃CH₂NH₂.

The term “alkenyl” when used without the “substituted” modifier refers to a monovalent group with a nonaromatic carbon atom as the point of attachment, a linear or branched, cyclo, cyclic or acyclic structure, at least one nonaromatic carbon-carbon double bond, no carbon-carbon triple bonds, and no atoms other than carbon and hydrogen. Non-limiting examples of alkenyl groups include: —CH═CH₂ (vinyl), —CH═CHCH₃, —CH═CHCH₂CH₃, —CH₂CH═CH₂ (allyl), —CH₂CH═CHCH₃, and —CH═CH—C₆H₅. The term “substituted alkenyl” refers to a monovalent group with a nonaromatic carbon atom as the point of attachment, at least one nonaromatic carbon-carbon double bond, no carbon-carbon triple bonds, a linear or branched, cyclo, cyclic or acyclic structure, and at least one atom independently selected from the group consisting of N, O, F, Cl, Br, I, Si, P, and S. The groups, —CH═CHF, —CH═CHCl and —CH═CHBr, are non-limiting examples of substituted alkenyl groups.

The term “alkenediyl” when used without the “substituted” modifier refers to a non-aromatic divalent group, wherein the alkenediyl group is attached with two σ-bonds, with two carbon atoms as points of attachment, a linear or branched, cyclo, cyclic or acyclic structure, at least one nonaromatic carbon-carbon double bond, no carbon-carbon triple bonds, and no atoms other than carbon and hydrogen. The groups, —CH═CH—, —CH═C(CH₃)CH₂—, —CH═CHCH₂—, and

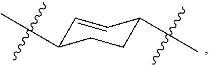 [SMILES: *C1C=CC(*)CC1]

are non-limiting examples of alkenediyl groups. The term “substituted alkenediyl” refers to a non-aromatic divalent group, wherein the alkenediyl group is attached with two σ-bonds, with two carbon atoms as points of attachment, a linear or branched, cyclo, cyclic or acyclic structure, at least one nonaromatic carbon-carbon double bond, no carbon-carbon triple bonds, and at least one atom independently selected from the group consisting of N, O, F, Cl, Br, I, Si, P, and S. The following groups are non-limiting examples of substituted alkenediyl groups: —CF═CH—, —C(OH)═CH—, and —CH₂CH═C(Cl)—.

The term “alkene” when used without the “substituted” modifier refers to a non-aromatic hydrocarbon having at least one carbon-carbon double bond and a linear or branched, cyclo, cyclic or acyclic structure. Thus, as used herein, cycloalkene is a subset of alkene. The compounds C₂H₄ (ethylene), CH₃CH═CH₂ (propene) and cylcohexene are non-limiting examples of alkenes. A “substituted alkene” differs from an alkene in that it also comprises at least one atom independently selected from the group consisting of N, O, F, Cl, Br, I, Si, P, and S.

The term “alkynyl” when used without the “substituted” modifier refers to a monovalent group with a nonaromatic carbon atom as the point of attachment, a linear or branched, cyclo, cyclic or acyclic structure, at least one carbon-carbon triple bond, and no atoms other than carbon and hydrogen. The groups, —C≡CH, —C≡CCH₃, —C≡CC₆H₅ and —CH₂C≡CCH₃, are non-limiting examples of alkynyl groups. The term “substituted alkynyl” refers to a monovalent group with a nonaromatic carbon atom as the point of attachment and at least one carbon-carbon triple bond, a linear or branched, cyclo, cyclic or acyclic structure, and at least one atom independently selected from the group consisting of N, O, F, Cl, Br, I, Si, P, and S. The group, —C≡CSi(CH₃)₃, is a non-limiting example of a substituted alkynyl group.

The term “alkynediyl” when used without the “substituted” modifier refers to a non-aromatic divalent group, wherein the alkynediyl group is attached with two σ-bonds, with two carbon atoms as points of attachment, a linear or branched, cyclo, cyclic or acyclic structure, at least one carbon-carbon triple bond, and no atoms other than carbon and hydrogen. The groups, —C≡C—, —C≡CCH₂—, and —C≡CCH(CH₃)— are non-limiting examples of alkynediyl groups. The term “substituted alkynediyl” refers to a non-aromatic divalent group, wherein the alkynediyl group is attached with two σ-bonds, with two carbon atoms as points of attachment, a linear or branched, cyclo, cyclic or acyclic structure, at least one carbon-carbon triple bond, and at least one atom independently selected from the group consisting of N, O, F, Cl, Br, I, Si, P, and S. The groups —C≡CCFH— and —C≡CHCH(Cl)— are non-limiting examples of substituted alkynediyl groups.

The term “alkyne” when used without the “substituted” modifier refers to a non-aromatic hydrocarbon having at least one carbon-carbon triple bond and a linear or branched, cyclo, cyclic or acyclic structure. Thus, as used herein, cycloalkene is a subset of alkene. The compounds C₂H₂ (acetylene), CH₃C≡CH (propene) and cylcooctyne are non-limiting examples of alkenes. A “substituted alkene” differs from an alkene in that it also comprises at least one atom independently selected from the group consisting of N, O, F, Cl, Br, I, Si, P, and S.

The term “aryl” when used without the “substituted” modifier refers to a monovalent group with an aromatic carbon atom as the point of attachment, said carbon atom forming part of one or more six-membered aromatic ring structure(s) wherein the ring atoms are all carbon, and wherein the monovalent group consists of no atoms other than carbon and hydrogen. Non-limiting examples of aryl groups include phenyl (Ph), methylphenyl, (dimethyl)phenyl, —C₆H₄CH₂CH₃ (ethylphenyl), —C₆H₄CH₂CH₂CH₃ (propylphenyl), —C₆H₄CH(CH₃)₂, —C₆H₄CH(CH₂)₂, —C₆H₃(CH₃)CH₂CH₃ (methylethylphenyl), —C₆H₄CH═CH₂ (vinylphenyl), —C₆H₄CH═CHCH₃, —C₆H₄C≡CH, —C₆H₄C≡CCH₃, naphthyl, and the monovalent group derived from biphenyl. The term “substituted aryl” refers to a monovalent group with an aromatic carbon atom as the point of attachment, said carbon atom forming part of one or more six-membered aromatic ring structure(s) wherein the ring atoms are all carbon, and wherein the monovalent group further has at least one atom independently selected from the group consisting of N, O, F, Cl, Br, I, Si, P, and S. Non-limiting examples of substituted aryl groups include the groups: —C₆H₄F, —C₆H₄Cl, —C₆H₄Br, —C₆H₄I, —C₆H₄OH, —C₆H₄OCH₃, —C₆H₄OCH₂CH₃, —C₆H₄OC(O)CH₃, —C₆H₄NH₂, —C₆H₄NHCH₃, —C₆H₄N(CH₃)₂, —C₆H₄CH₂OH, —C₆H₄CH₂OC(O)CH₃, —C₆H₄CH₂NH₂, —C₆H₄CF₃, —C₆H₄CN, —C₆H₄CHO, —C₆H₄CHO, —C₆H₄C(O)CH₃, —C₆H₄C(O)C₆H₅, —C₆H₄CO₂H, —C₆H₄CO₂CH₃, —C₆H₄CONH₂, —C₆H₄CONHCH₃, and —C₆H₄CON(CH₃)₂.

The term “arenediyl” when used without the “substituted” modifier refers to a divalent group, wherein the arenediyl group is attached with two σ-bonds, with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. Non-limiting examples of arenediyl groups include:

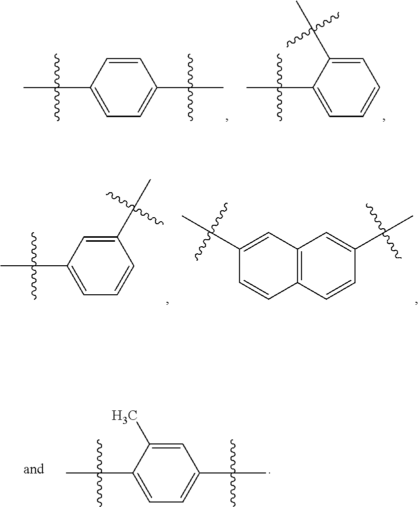

The term “substituted arenediyl” refers to a divalent group, wherein the arenediyl group is attached with two σ-bonds, with two aromatic carbon atoms as points of attachment, said carbon atoms forming part of one or more six-membered aromatic rings structure(s), wherein the ring atoms are carbon, and wherein the divalent group further has at least one atom independently selected from the group consisting of N, O, F, Cl, Br, I, Si, P, and S.

The term “arene” when used without the “substituted” modifier refers to an hydrocarbon having at least one six-membered aromatic ring. One or more alkyl, alkenyl or alkynyl groups may be optionally attached to this ring. Also this ring may optionally be fused with other rings, including non-aromatic rings. Benzene, toluene, naphthalene, and biphenyl are non-limiting examples of arenes. A “substituted arene” differs from an arene in that it also comprises at least one atom independently selected from the group consisting of N, O, F, Cl, Br, I, Si, P, and S. Phenol and nitrobenzene are non-limiting examples of substituted arenes.

The term “aralkyl” when used without the “substituted” modifier refers to the monovalent group -alkanediyl-aryl, in which the terms alkanediyl and aryl are each used in a manner consistent with the definitions provided above. Non-limiting examples of aralkyls are: phenylmethyl (benzyl, Bn), 1-phenyl-ethyl, 2-phenyl-ethyl, indenyl and 2,3-dihydro-indenyl, provided that indenyl and 2,3-dihydro-indenyl are only examples of aralkyl in so far as the point of attachment in each case is one of the saturated carbon atoms. When the term “aralkyl” is used with the “substituted” modifier, either one or both the alkanediyl and the aryl is substituted. Non-limiting examples of substituted aralkyls are: (3-chlorophenyl)-methyl, 2-oxo-2-phenyl-ethyl (phenylcarbonylmethyl), 2-chloro-2-phenyl-ethyl, chromanyl where the point of attachment is one of the saturated carbon atoms, and tetrahydroquinolinyl where the point of attachment is one of the saturated atoms.

The term “heteroaryl” when used without the “substituted” modifier refers to a monovalent group with an aromatic carbon atom or nitrogen atom as the point of attachment, said carbon atom or nitrogen atom forming part of an aromatic ring structure wherein at least one of the ring atoms is nitrogen, oxygen or sulfur, and wherein the monovalent group consists of no atoms other than carbon, hydrogen, aromatic nitrogen, aromatic oxygen and aromatic sulfur. Non-limiting examples of aryl groups include acridinyl, furanyl, imidazoimidazolyl, imidazopyrazolyl, imidazopyridinyl, imidazopyrimidinyl, indolyl, indazolinyl, methylpyridyl, oxazolyl, phenylimidazolyl, pyridyl, pyrrolyl, pyrimidyl, pyrazinyl, quinolyl, quinazolyl, quinoxalinyl, tetrahydroquinolinyl, thienyl, triazinyl, pyrrolopyridinyl, pyrrolopyrimidinyl, pyrrolopyrazinyl, pyrrolotriazinyl, pyrroloimidazolyl, chromenyl (where the point of attachment is one of the aromatic atoms), and chromanyl (where the point of attachment is one of the aromatic atoms). The term “substituted heteroaryl” refers to a monovalent group with an aromatic carbon atom or nitrogen atom as the point of attachment, said carbon atom or nitrogen atom forming part of an aromatic ring structure wherein at least one of the ring atoms is nitrogen, oxygen or sulfur, and wherein the monovalent group further has at least one atom independently selected from the group consisting of non-aromatic nitrogen, non-aromatic oxygen, non aromatic sulfur F, Cl, Br, I, Si, and P.

The term “heteroarenediyl” when used without the “substituted” modifier refers to a divalent group, wherein the heteroarenediyl group is attached with two σ-bonds, with an aromatic carbon atom or nitrogen atom as the point of attachment, said carbon atom or nitrogen atom forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. Non-limiting examples of heteroarenediyl groups include:

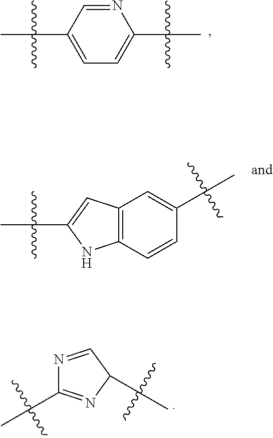 [SMILES: *c1ccc(*)nc1 ; *c1ccc2[nH]c(*)cc2c1 ; *C1=NC(*)C=N1]

Specific examples of heteroarenediyl groups contemplated by the present disclosure include, but are not limited to purine, quinoline, quninolinium, pyridine, pyridinium, pyrimidine, imidazole, pyrazine, triazole, 1,2,3-triazole, 1,2,4-triazone and derivatives thereof.

The term “substituted heteroarenediyl” refers to a divalent group, wherein the heteroarenediyl group is attached with two σ-bonds, with an aromatic carbon atom or nitrogen atom as points of attachment, said carbon atom or nitrogen atom forming part of one or more six-membered aromatic ring structure(s), wherein at least one of the ring atoms is nitrogen, oxygen or sulfur, and wherein the divalent group further has at least one atom independently selected from the group consisting of non-aromatic nitrogen, non-aromatic oxygen, non aromatic sulfur F, Cl, Br, I, Si, and P. Specific examples of substituted heteroarenediyl groups contemplated by the present disclosure include, but are not limited to purine, quinoline, quninolinium, pyridine, pyridinium, pyrimidine, imidazole, pyrazine, triazole, 1,2,3-triazole, 1,2,4-triazone and derivatives thereof. In some examples, the substituted heteroarenediyl is functionalized by an electron withdrawing group. Particular examples of electron withdrawing groups include, but are not limited to —Cl, —F, —Br, —NO₂, —COOR (carboxylate), —COR (acyl), —CN, —SO₂R (sulfone), —SO₂NR₁R₂ (sulfamide), —P(O)(OR)₂ wherein R, R₁, and R₂ are independently selected from alkyl, alkoxy, alkene, etc.

The term “heteroaralkyl” when used without the “substituted” modifier refers to the monovalent group -alkanediyl-heteroaryl, in which the terms alkanediyl and heteroaryl are each used in a manner consistent with the definitions provided above. Non-limiting examples of aralkyls are: pyridylmethyl, and thienylmethyl. When the term “heteroaralkyl” is used with the “substituted” modifier, either one or both the alkanediyl and the heteroaryl is substituted.

The term “acyl” when used without the “substituted” modifier refers to a monovalent group with a carbon atom of a carbonyl group as the point of attachment, further having a linear or branched, cyclo, cyclic or acyclic structure, further having no additional atoms that are not carbon or hydrogen, beyond the oxygen atom of the carbonyl group. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)C₆H₄CH₂CH₃, —COC₆H₃(CH₃)₂, and —C(O)CH₂C₆H₅, are non-limiting examples of acyl groups. The term “acyl” therefore encompasses, but is not limited to groups sometimes referred to as “alkyl carbonyl” and “aryl carbonyl” groups. The term “substituted acyl” refers to a monovalent group with a carbon atom of a carbonyl group as the point of attachment, further having a linear or branched, cyclo, cyclic or acyclic structure, further having at least one atom, in addition to the oxygen of the carbonyl group, independently selected from the group consisting of N, O, F, Cl, Br, I, Si, P, and S. The groups, —C(O)CH₂CF₃, —CO₂H (carboxyl), —CO₂CH₃ (methylcarboxyl), —CO₂CH₂CH₃, —CO₂CH₂CH₂CH₃, —CO₂C₆H₅, —CO₂CH(CH₃)₂, —CO₂CH(CH₂)₂, —C(O)NH₂ (carbamoyl), —C(O)NHCH₃, —C(O)NHCH₂CH₃, —CONHCH(CH₃)₂, —CONHCH(CH₂)₂, —CON(CH₃)₂, —CONHCH₂CF₃, —CO-pyridyl, —CO-imidazoyl, and —C(O)N₃, are non-limiting examples of substituted acyl groups. The term “substituted acyl” encompasses, but is not limited to, “heteroaryl carbonyl” groups.

The term “alkylidene” when used without the “substituted” modifier refers to the divalent group ═CRR′, wherein the alkylidene group is attached with one σ-bond and one π-bond, in which R and R′ are independently hydrogen, alkyl, or R and R′ are taken together to represent alkanediyl. Non-limiting examples of alkylidene groups include: ═CH₂, ═CH(CH₂CH₃), and ═C(CH₃)₂. The term “substituted alkylidene” refers to the group ═CRR′, wherein the alkylidene group is attached with one σ-bond and one π-bond, in which R and R′ are independently hydrogen, alkyl, substituted alkyl, or R and R′ are taken together to represent a substituted alkanediyl, provided that either one of R and R′ is a substituted alkyl or R and R′ are taken together to represent a substituted alkanediyl.

The term “alkoxy” when used without the “substituted” modifier refers to the group —OR, in which R is an alkyl, as that term is defined above. Non-limiting examples of alkoxy groups include: —OCH₃, —OCH₂CH₃, —OCH₂CH₂CH₃, —OCH(CH₃)₂, —OCH(CH₂)₂, —O-cyclopentyl, and —O-cyclohexyl. The term “substituted alkoxy” refers to the group —OR, in which R is a substituted alkyl, as that term is defined above. For example, —OCH₂CF₃ is a substituted alkoxy group.

The term “alcohol” when used without the “substituted” modifier corresponds to an alkane, as defined above, wherein at least one of the hydrogen atoms has been replaced with a hydroxy group. Alcohols have a linear or branched, cyclo, cyclic or acyclic structure. The compounds methanol, ethanol and cyclohexanol are non-limiting examples of alcohols. A “substituted alkane” differs from an alcohol in that it also comprises at least one atom independently selected from the group consisting of N, F, Cl, Br, I, Si, P, and S.

Similarly, the terms “alkenyloxy”, “alkynyloxy”, “aryloxy”, “aralkoxy”, “heteroaryloxy”, “heteroaralkoxy” and “acyloxy”, when used without the “substituted” modifier, refers to groups, defined as —OR, in which R is alkenyl, alkynyl, aryl, aralkyl, heteroaryl, heteroaralkyl and acyl, respectively, as those terms are defined above. When any of the terms alkenyloxy, alkynyloxy, aryloxy, aralkyloxy and acyloxy is modified by “substituted,” it refers to the group —OR, in which R is substituted alkenyl, alkynyl, aryl, aralkyl, heteroaryl, heteroaralkyl and acyl, respectively.

The term “alkylamino” when used without the “substituted” modifier refers to the group —NHR, in which R is an alkyl, as that term is defined above. Non-limiting examples of alkylamino groups include: —NHCH₃, —NHCH₂CH₃, —NHCH₂CH₂CH₃, —NHCH(CH₃)₂, —NHCH(CH₂)₂, —NHCH₂CH₂CH₂CH₃, —NHCH(CH₃)CH₂CH₃, —NHCH₂CH(CH₃)₂, —NHC(CH₃)₃, —NH-cyclopentyl, and —NH-cyclohexyl. The term “substituted alkylamino” refers to the group —NHR, in which R is a substituted alkyl, as that term is defined above. For example, —NHCH₂CF₃ is a substituted alkylamino group.

The term “dialkylamino” when used without the “substituted” modifier refers to the group —NRR′, in which R and R′ can be the same or different alkyl groups, or R and R′ can be taken together to represent an alkanediyl having two or more saturated carbon atoms, at least two of which are attached to the nitrogen atom. Non-limiting examples of dialkylamino groups include: —NHC(CH₃)₃, —N(CH₃)CH₂CH₃, —N(CH₂CH₃)₂, N-pyrrolidinyl, and N-piperidinyl. The term “substituted dialkylamino” refers to the group —NRR′, in which R and R′can be the same or different substituted alkyl groups, one of R or R′ is an alkyl and the other is a substituted alkyl, or R and R′ can be taken together to represent a substituted alkanediyl with two or more saturated carbon atoms, at least two of which are attached to the nitrogen atom.

The terms “alkoxyamino”, “alkenylamino”, “alkynylamino”, “arylamino”, “aralkylamino”, “heteroarylamino”, “heteroaralkylamino”, and “alkylsulfonylamino” when used without the “substituted” modifier, refers to groups, defined as —NHR, in which R is alkoxy, alkenyl, alkynyl, aryl, aralkyl, heteroaryl, heteroaralkyl and alkylsulfonyl, respectively, as those terms are defined above. A non-limiting example of an arylamino group is —NHC₆H₅. When any of the terms alkoxyamino, alkenylamino, alkynylamino, arylamino, aralkylamino, heteroarylamino, heteroaralkylamino and alkylsulfonylamino is modified by “substituted,” it refers to the group —NHR, in which R is substituted alkoxy, alkenyl, alkynyl, aryl, aralkyl, heteroaryl, heteroaralkyl and alkylsulfonyl, respectively.

The term “amido” (acylamino), when used without the “substituted” modifier, refers to the group —NHR, in which R is acyl, as that term is defined above. A non-limiting example of an acylamino group is —NHC(O)CH₃. When the term amido is used with the “substituted” modifier, it refers to groups, defined as —NHR, in which R is substituted acyl, as that term is defined above. The groups —NHC(O)OCH₃ and —NHC(O)NHCH₃ are non-limiting examples of substituted amido groups.

The term “alkylimino” when used without the “substituted” modifier refers to the group ═NR, wherein the alkylimino group is attached with one σ-bond and one π-bond, in which R is an alkyl, as that term is defined above. Non-limiting examples of alkylimino groups include: ═NCH₃, ═NCH₂CH₃ and ═N-cyclohexyl. The term “substituted alkylimino” refers to the group ═NR, wherein the alkylimino group is attached with one σ-bond and one π-bond, in which R is a substituted alkyl, as that term is defined above. For example, ═NCH₂CF₃ is a substituted alkylimino group.

Similarly, the terms “alkenylimino”, “alkynylimino”, “arylimino”, “aralkylimino”, “heteroarylimino”, “heteroaralkylimino” and “acylimino”, when used without the “substituted” modifier, refers to groups, defined as ═NR, wherein the alkylimino group is attached with one σ-bond and one π-bond, in which R is alkenyl, alkynyl, aryl, aralkyl, heteroaryl, heteroaralkyl and acyl, respectively, as those terms are defined above. When any of the terms alkenylimino, alkynylimino, arylimino, aralkylimino and acylimino is modified by “substituted,” it refers to the group ═NR, wherein the alkylimino group is attached with one π-bond and one π-bond, in which R is substituted alkenyl, alkynyl, aryl, aralkyl, heteroaryl, heteroaralkyl and acyl, respectively.

The term “fluoroalkyl” when used without the “substituted” modifier refers to an alkyl, as that term is defined above, in which one or more fluorines have been substituted for hydrogens. The groups, —CH₂F, —CF₂H, —CF₃, and —CH₂CF₃ are non-limiting examples of fluoroalkyl groups. The term “substituted fluoroalkyl” refers to a non-aromatic monovalent group with a saturated carbon atom as the point of attachment, a linear or branched, cyclo, cyclic or acyclic structure, at least one fluorine atom, no carbon-carbon double or triple bonds, and at least one atom independently selected from the group consisting of N, O, Cl, Br, I, Si, P, and S. The following group is a non-limiting example of a substituted fluoroalkyl: —CFHOH.

The term “alkylphosphate” when used without the “substituted” modifier refers to the group —OP(O)(OH)(OR), in which R is an alkyl, as that term is defined above. Non-limiting examples of alkylphosphate groups include: —OP(O)(OH)(OMe) and —OP(O)(OH)(OEt). The term “substituted alkylphosphate” refers to the group —OP(O)(OH)(OR), in which R is a substituted alkyl, as that term is defined above.

The term “dialkylphosphate” when used without the “substituted” modifier refers to the group —OP(O)(OR)(OR′), in which R and R′ can be the same or different alkyl groups, or R and R′ can be taken together to represent an alkanediyl having two or more saturated carbon atoms, at least two of which are attached via the oxygen atoms to the phosphorus atom. Non-limiting examples of dialkylphosphate groups include: —OP(O)(OMe)₂, —OP(O)(OEt)(OMe) and —OP(O)(OEt)₂. The term “substituted dialkylphosphate” refers to the group —OP(O)(OR)(OR′), in which R and R′ can be the same or different substituted alkyl groups, one of R or R′ is an alkyl and the other is a substituted alkyl, or R and R′ can be taken together to represent a substituted alkanediyl with two or more saturated carbon atoms, at least two of which are attached via the oxygen atoms to the phosphorous.

The term “alkylthio” when used without the “substituted” modifier refers to the group —SR, in which R is an alkyl, as that term is defined above. Non-limiting examples of alkylthio groups include: —SCH₃, —SCH₂CH₃, —SCH₂CH₂CH₃, —SCH(CH₃)₂, —SCH(CH₂)₂, —S-cyclopentyl, and —S-cyclohexyl. The term “substituted alkylthio” refers to the group —SR, in which R is a substituted alkyl, as that term is defined above. For example, —SCH₂CF₃ is a substituted alkylthio group.

Similarly, the terms “alkenylthio”, “alkynylthio”, “arylthio”, “aralkylthio”, “heteroarylthio”, “heteroaralkylthio”, and “acylthio”, when used without the “substituted” modifier, refers to groups, defined as —SR, in which R is alkenyl, alkynyl, aryl, aralkyl, heteroaryl, heteroaralkyl and acyl, respectively, as those terms are defined above. When any of the terms alkenylthio, alkynylthio, arylthio, aralkylthio, heteroarylthio, heteroaralkylthio, and acylthio is modified by “substituted,” it refers to the group —SR, in which R is substituted alkenyl, alkynyl, aryl, aralkyl, heteroaryl, heteroaralkyl and acyl, respectively.

The term “thioacyl” when used without the “substituted” modifier refers to a monovalent group with a carbon atom of a thiocarbonyl group as the point of attachment, further having a linear or branched, cyclo, cyclic or acyclic structure, further having no additional atoms that are not carbon or hydrogen, beyond the sulfur atom of the carbonyl group. The groups, —CHS, —C(S)CH₃, —C(S)CH₂CH₃, —C(S)CH₂CH₂CH₃, —C(S)CH(CH₃)₂, —C(S)CH(CH₂)₂, —C(S)C₆H₅, —C(S)C₆H₄CH₃, —C(S)C₆H₄CH₂CH₃, —C(S)C₆H₃(CH₃)₂, and —C(S)CH₂C₆H₅, are non-limiting examples of thioacyl groups. The term “thioacyl” therefore encompasses, but is not limited to, groups sometimes referred to as “alkyl thiocarbonyl” and “aryl thiocarbonyl” groups. The term “substituted thioacyl” refers to a radical with a carbon atom as the point of attachment, the carbon atom being part of a thiocarbonyl group, further having a linear or branched, cyclo, cyclic or acyclic structure, further having at least one atom, in addition to the sulfur atom of the carbonyl group, independently selected from the group consisting of N, O, F, Cl, Br, I, Si, P, and S. The groups, —C(S)CH₂CF₃, —C(S)O₂H, —C(S)OCH₃, —C(S)OCH₂CH₃, —C(S)OCH₂CH₂CH₃, —C(S)OC₆H₅, —C(S)OCH(CH₃)₂, —C(S)OCH(CH₂)₂, —C(S)NH₂, and —C(S)NHCH₃, are non-limiting examples of substituted thioacyl groups. The term “substituted thioacyl” encompasses, but is not limited to, “heteroaryl thiocarbonyl” groups.

The term “alkylsulfonyl” when used without the “substituted” modifier refers to the group —S(O)₂R, in which R is an alkyl, as that term is defined above. Non-limiting examples of alkylsulfonyl groups include: —S(O)₂CH₃, —S(O)₂CH₂CH₃, —S(O)₂CH₂CH₂CH₃, —S(O)₂CH(CH₃)₂, —S(O)₂CH(CH₂)₂, —S(O)₂— cyclopentyl, and —S(O)₂— cyclohexyl. The term “substituted alkylsulfonyl” refers to the group —S(O)₂R, in which R is a substituted alkyl, as that term is defined above. For example, —S(O)₂CH₂CF₃ is a substituted alkylsulfonyl group.

Similarly, the terms “alkenylsulfonyl”, “alkynylsulfonyl”, “arylsulfonyl”, “aralkylsulfonyl”, “heteroarylsulfonyl”, and “heteroaralkylsulfonyl” when used without the “substituted” modifier, refers to groups, defined as —S(O)₂R, in which R is alkenyl, alkynyl, aryl, aralkyl, heteroaryl, and heteroaralkyl, respectively, as those terms are defined above. When any of the terms alkenylsulfonyl, alkynylsulfonyl, arylsulfonyl, aralkylsulfonyl, heteroarylsulfonyl, and heteroaralkylsulfonyl is modified by “substituted,” it refers to the group —S(O)₂R, in which R is substituted alkenyl, alkynyl, aryl, aralkyl, heteroaryl and heteroaralkyl, respectively.

The term “alkylsulfinyl” when used without the “substituted” modifier refers to the group —S(O)R, in which R is an alkyl, as that term is defined above. Non-limiting examples of alkylsulfinyl groups include: —S(O)CH₃, —S(O)CH₂CH₃, —S(O)CH₂CH₂CH₃, —S(O)CH(CH₃)₂, —S(O)CH(CH₂)₂, —S(O)— cyclopentyl, and —S(O)— cyclohexyl. The term “substituted alkylsulfinyl” refers to the group —S(O)R, in which R is a substituted alkyl, as that term is defined above. For example, —S(O)CH₂CF₃ is a substituted alkylsulfinyl group.

Similarly, the terms “alkenylsulfinyl”, “alkynylsulfinyl”, “arylsulfinyl”, “aralkylsulfinyl”, “heteroarylsulfinyl”, and “heteroaralkylsulfinyl” when used without the “substituted” modifier, refers to groups, defined as —S(O)R, in which R is alkenyl, alkynyl, aryl, aralkyl, heteroaryl, and heteroaralkyl, respectively, as those terms are defined above. When any of the terms alkenylsulfinyl, alkynylsulfinyl, arylsulfinyl, aralkylsulfinyl, heteroarylsulfinyl, and heteroaralkylsulfinyl is modified by “substituted,” it refers to the group —S(O)R, in which R is substituted alkenyl, alkynyl, aryl, aralkyl, heteroaryl and heteroaralkyl, respectively.

The term “alkylammonium” when used without the “substituted” modifier refers to a group, defined as —NH₂R⁺, —NHRR′⁺, or —NRR′R″⁺, in which R, R′ and R″ are the same or different alkyl groups, or any combination of two of R, R′ and R″ can be taken together to represent an alkanediyl. Non-limiting examples of alkylammonium cation groups include: —NH₂(CH₃)⁺, —NH₂(CH₂CH₃)⁺, —NH₂(CH₂CH₂CH₃)⁺, —NH(CH₃)₂⁺, —NH(CH₂CH₃)₂⁺, —NH(CH₂CH₂CH₃)₂⁺, —N(CH₃)₃⁺, —N(CH₃)(CH₂CH₃)₂⁺, —N(CH₃)₂(CH₂CH₃)⁺, —NH₂C(CH₃)₃⁺, —NH(cyclopentyl)₂⁺, and —NH₂(cyclohexyl)⁺. The term “substituted alkylammonium” refers —NH₂R⁺, —NHRR′⁺, or —NRR′R″⁺, in which at least one of R, R′ and R″ is a substituted alkyl or two of R, R′ and R″ can be taken together to represent a substituted alkanediyl. When more than one of R, R′ and R″ is a substituted alkyl, they can be the same of different. Any of R, R′ and R″ that are not either substituted alkyl or substituted alkanediyl, can be either alkyl, either the same or different, or can be taken together to represent a alkanediyl with two or more carbon atoms, at least two of which are attached to the nitrogen atom shown in the formula.

The term “alkylsulfonium” when used without the “substituted” modifier refers to the group —SRR′, in which R and R′ can be the same or different alkyl groups, or R and R′ can be taken together to represent an alkanediyl. Non-limiting examples of alkylsulfonium groups include: —SH(CH₃)⁺, —SH(CH₂CH₃)⁺, —SH(CH₂CH₂CH₃)⁺, —S(CH₃)₂⁺, —S(CH₂CH₃)₂⁺, —S(CH₂CH₂CH₃)₂⁺, —SH(cyclopentyl)⁺, and —SH(cyclohexyl)⁺. The term “substituted alkylsulfonium” refers to the group —SRR′, in which R and R′ can be the same or different substituted alkyl groups, one of R or R′ is an alkyl and the other is a substituted alkyl, or R and R′ can be taken together to represent a substituted alkanediyl. For example, —SH(CH₂CF₃)⁺ is a substituted alkylsulfonium group.

The term “alkylsilyl” when used without the “substituted” modifier refers to a monovalent group, defined as —SiH₂R, —SiHRR′, or —SiRR′R″, in which R, R′ and R″ can be the same or different alkyl groups, or any combination of two of R, R′ and R″ can be taken together to represent an alkanediyl. The groups, —SiH₂CH₃, —SiH(CH₃)₂, —Si(CH₃)₃ and —Si(CH₃)₂C(CH₃)₃, are non-limiting examples of unsubstituted alkylsilyl groups. The term “substituted alkylsilyl” refers to —SiH₂R, —SiHRR′, or —SiRR′R″, in which at least one of R, R′ and R″ is a substituted alkyl or two of R, R′ and R″ can be taken together to represent a substituted alkanediyl. When more than one of R, R′ and R″ is a substituted alkyl, they can be the same of different. Any of R, R′ and R″ that are not either substituted alkyl or substituted alkanediyl, can be either alkyl, either the same or different, or can be taken together to represent a alkanediyl with two or more saturated carbon atoms, at least two of which are attached to the silicon atom.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C. Similarly, it is contemplated that one or more carbon atom(s) of a compound of the present invention may be replaced by a silicon atom(s). Furthermore, it is contemplated that one or more oxygen atom(s) of a compound of the present invention may be replaced by a sulfur or selenium atom(s).

A compound having a formula that is represented with a dashed bond is intended to include the formulae optionally having zero, one or more double bonds. Thus, for example, the structure

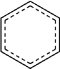

includes the structures

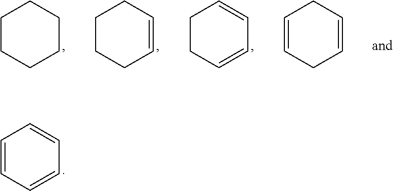

As will be understood by a person of skill in the art, no one such ring atom forms part of more than one double bond.

Any undefined valency on an atom of a structure shown in this application implicitly represents a hydrogen atom bonded to the atom.

As used herein, a “chiral auxiliary” refers to a removable chiral group that is capable of influencing the stereoselectivity of a reaction. Persons of skill in the art are familiar with such compounds, and many are commercially available.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human subjects are adults, juveniles, infants and fetuses.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

As used herein, “predominantly one enantiomer” means that a compound contains at least about 85% of one enantiomer, or more preferably at least about 90% of one enantiomer, or even more preferably at least about 95% of one enantiomer, or most preferably at least about 99% of one enantiomer. Similarly, the phrase “substantially free from other optical isomers” means that the composition contains at most about 15% of another enantiomer or diastereomer, more preferably at most about 10% of another enantiomer or diastereomer, even more preferably at most about 5% of another enantiomer or diastereomer, and most preferably at most about 1% of another enantiomer or diastereomer.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an inhibitor according to the present invention. The prodrug itself may or may not also have activity with respect to a given target protein. For example, a compound comprising a hydroxy group may be administered as an ester that is converted by hydrolysis in vivo to the hydroxy compound. Suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methanesulfonates, ethanesulfonates, benzene sulfonates, p-toluenesulfonates, cyclohexylsulfamates, quinates, esters of amino acids, and the like. Similarly, a compound comprising an amine group may be administered as an amide that is converted by hydrolysis in vivo to the amine compound.

A “repeat unit” is the simplest structural entity of certain materials, for example, frameworks and/or polymers, whether organic, inorganic or metal-organic. In the case of a polymer chain, repeat units are linked together successively along the chain, like the beads of a necklace. For example, in polyethylene, —[—CH₂CH₂—]ₙ—, the repeat unit is —CH₂CH₂—. The subscript “n” denotes the degree of polymerisation, that is, the number of repeat units linked together. When the value for “n” is left undefined, it simply designates repetition of the formula within the brackets as well as the polymeric nature of the material. The concept of a repeat unit applies equally to where the connectivity between the repeat units extends three dimensionally, such as in metal organic frameworks, cross-linked polymers, thermosetting polymers, etc.

The term “saturated” when referring to an atom means that the atom is connected to other atoms only by means of single bonds.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are minor images of each other, like left and right hands. “Diastereomers” are stereoisomers of a given compound that are not enantiomers.

The invention contemplates that for any stereocenter or axis of chirality for which stereochemistry has not been defined, that stereocenter or axis of chirality can be present in its R form, S form, or as a mixture of the R and S forms, including racemic and non-racemic mixtures.

“Substituent convertible to hydrogen in vivo” means any group that is convertible to a hydrogen atom by enzymological or chemical means including, but not limited to, hydrolysis and hydrogenolysis. Examples include hydrolyzable groups, such as acyl groups, groups having an oxycarbonyl group, amino acid residues, peptide residues, o-nitrophenylsulfenyl, trimethylsilyl, tetrahydropyranyl, diphenylphosphinyl, and the like. Examples of acyl groups include formyl, acetyl, trifluoroacetyl, and the like. Examples of groups having an oxycarbonyl group include ethoxycarbonyl, tert-butoxycarbonyl (—C(O)OC(CH₃)₃), benzyloxycarbonyl, p-methoxybenzyloxycarbonyl, vinyloxycarbonyl, β-(p-toluenesulfonyl)ethoxycarbonyl, and the like. Suitable amino acid residues include, but are not limited to, residues of Gly (glycine), Ala (alanine), Arg (arginine), Asn (asparagine), Asp (aspartic acid), Cys (cysteine), Glu (glutamic acid), His (histidine), Ile (isoleucine), Leu (leucine), Lys (lysine), Met (methionine), Phe (phenylalanine), Pro (proline), Ser (serine), Thr (threonine), Trp (tryptophan), Tyr (tyrosine), Val (valine), Nva (norvaline), Hse (homoserine), 4-Hyp (4-hydroxyproline), 5-Hyl (5-hydroxylysine), Orn (ornithine) and β-Ala. Examples of suitable amino acid residues also include amino acid residues that are protected with a protecting group. Examples of suitable protecting groups include those typically employed in peptide synthesis, including acyl groups (such as formyl and acetyl), arylmethyloxycarbonyl groups (such as benzyloxycarbonyl and p-nitrobenzyloxycarbonyl), tert-butoxycarbonyl groups (—C(O)OC(CH₃)₃), and the like. Suitable peptide residues include peptide residues comprising two to five amino acid residues. The residues of these amino acids or peptides can be present in stereochemical configurations of the D-form, the L-form or mixtures thereof. In addition, the amino acid or peptide residue may have an asymmetric carbon atom. Examples of suitable amino acid residues having an asymmetric carbon atom include residues of Ala, Leu, Phe, Trp, Nva, Val, Met, Ser, Lys, Thr and Tyr. Peptide residues having an asymmetric carbon atom include peptide residues having one or more constituent amino acid residues having an asymmetric carbon atom. Examples of suitable amino acid protecting groups include those typically employed in peptide synthesis, including acyl groups (such as formyl and acetyl), arylmethyloxycarbonyl groups (such as benzyloxycarbonyl and p-nitrobenzyloxycarbonyl), tert-butoxycarbonyl groups (—C(O)OC(CH₃)₃), and the like. Other examples of substituents “convertible to hydrogen in vivo” include reductively eliminable hydrogenolyzable groups. Examples of suitable reductively eliminable hydrogenolyzable groups include, but are not limited to, arylsulfonyl groups (such as o-toluenesulfonyl); methyl groups substituted with phenyl or benzyloxy (such as benzyl, trityl and benzyloxymethyl); arylmethoxycarbonyl groups (such as benzyloxycarbonyl and o-methoxy-benzyloxycarbonyl); and haloethoxycarbonyl groups (such as β,β,β-trichloroethoxycarbonyl and β-iodoethoxycarbonyl).

“Effective amount,” “Therapeutically effective amount” or “pharmaceutically effective amount” means that amount which, when administered to a subject or patient for treating a disease, is sufficient to effect such treatment for the disease.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

As used herein, the term “water soluble” means that the compound dissolves in water at least to the extent of 0.010 mole/liter or is classified as soluble according to literature precedence.

Other abbreviations used herein are as follows: DMSO, dimethyl sulfoxide; NO, nitric oxide; iNOS, inducible nitric oxide synthase; COX-2, cyclooxygenase-2; NGF, nerve growth factor; IBMX, isobutylmethylxanthine; FBS, fetal bovine serum; GPDH, glycerol 3-phosphate dehydrogenase; RXR, retinoid X receptor; TGF-β, transforming growth factor-β; IFNγ or IFN-γ, interferon-γ; LPS, bacterial endotoxic lipopolysaccharide; TNFα or TNF-α, tumor necrosis factor-α; IL-1β, interleukin-1β; GAPDH, glyceraldehyde-3-phosphate dehydrogenase; MTT, 3-[4,5-dimethylthiazol-2-yl]-2,5-diphenyltetrazolium bromide; TCA, trichloroacetic acid; HO-1, inducible heme oxygenase.

The above definitions supersede any conflicting definition in any of the reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### II. Pharmaceutical Preparations

Pharmaceutical compositions of the present invention comprise an effective amount of one or more antimicrobial compositions dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one antimicrobial composition will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The antimicrobial composition may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The antimicrobial composition may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include the antimicrobial composition, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the antimicrobial composition may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

A. Alimentary Compositions and Formulations

In preferred embodiments of the present invention, the antimicrobial composition is formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792,451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

B. Parenteral Compositions and Formulations

In further embodiments, the antimicrobial composition may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

C. Miscellaneous Pharmaceutical Compositions and Formulations

In other preferred embodiments of the invention, the active compound antimicrobial composition may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### III. Exemplary Chemical Compositions

In certain embodiments of the invention, the tryptophan residue Trp211 plays an important role in recognizing DXR inhibitors. Therefore, in at least certain cases it is desirous for the disclosed inhibitors to have an electron-deficient hydrophobic group that has strong interactions with Trp211. Since, Trp211 is conserved across the species using the MEP pathway, in certain embodiments inhibitors having strong interactions with Trp211 would exhibit a broad spectrum of activity.

In some embodiments, the structures described herein are useful to probe the hydrophobic binding site of DXR. In particular embodiments, the DXR inhibitor contains an electron-deficient heterocyclic rings that is designed specifically to interact with the electron-rich indole ring of Trp211. Examples of an electron-deficient heterocyclic rings include, but are not limited to pyridine, quinoline, pyridinium, quinolinium, pyrimidine, purine, imidazolium, pyrazine, triazole, and in an even more general sense aromatic rings containing at least one nitrogen atom. In some examples, the aromatic ring contains at least two nitrogen atoms.

For example, embodiments of the invention encompass the structure having the general formula

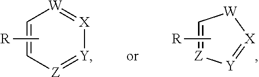

In some examples, W is either a nitrogen atom or CRi. In some examples, X is either a nitrogen atom or CR₂. In some examples, Y is either a nitrogen atom or CR₃. In some examples, Z is either a nitrogen atom or CR₄. In general the heterocyclic electron deficient ring is a heteroarenediyl group that includes, but is not limited to purine, quinoline, quninolinium, pyridine, pyridinium, pyrimidine, imidazole, pyrazine, triazole, 1,2,3-triazole, 1,2,4-triazone and derivatives thereof. In certain examples, W, X, Y and Z form a heteroarenediyl group. In particular examples, W, X, Y and Z form a purine, a quinoline, a quninolinium, a pyridine, a pyridinium, a pyrimidine, am imidazole, a pyrazine, a triazole, a 1,2,3-triazole, a 1,2,4-triazone or a derivative thereof.

In general the DXR inhibitor can have any one of the following general structures.

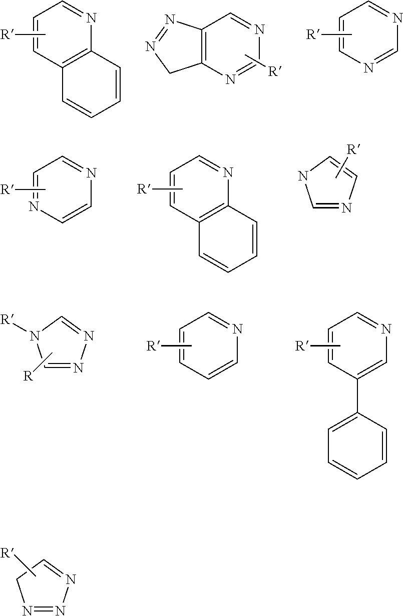

In the above examples, R′ is one of the following:

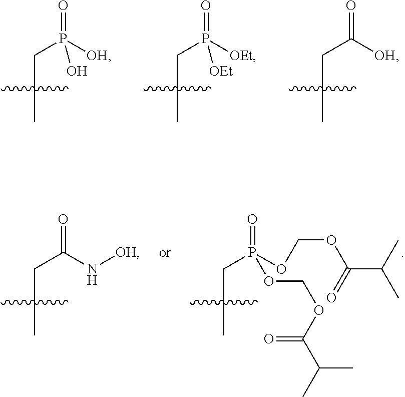 [SMILES: *CP(=O)(O)O ; *CP(=O)(OCC)OCC ; *CC(=O)O ; *CC(=O)NO ; *CP(=O)(OCOC(=O)C(C)C)OCOC(=O)C(C)C]

As a specific example, in the following structures, W, X, Y and Z form a heteroarenediyl group with R′ having the formula

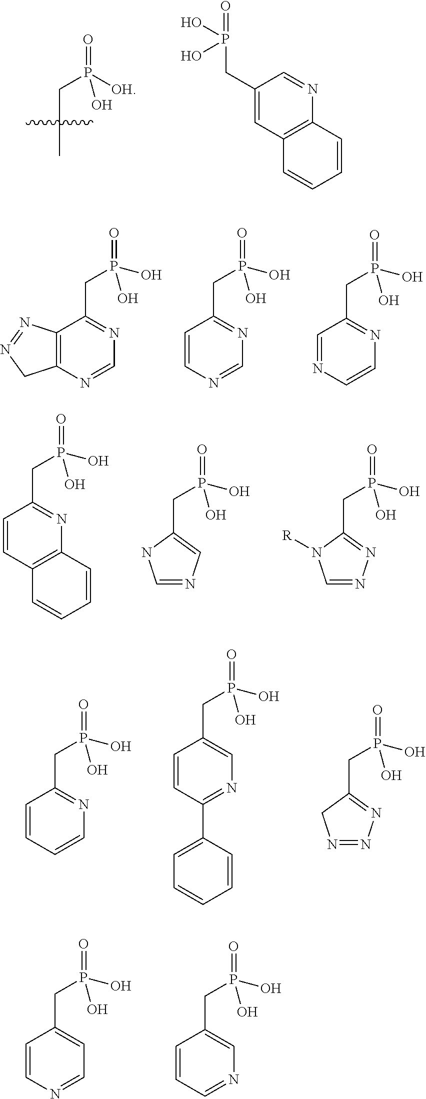

In some examples, the heteroarenediyl group is further functionalized with an electron withdrawing group. Specific examples of an electron withdrawing group include, but are not limited to —Cl, —F, —Br, —NO₂, —COOR (carboxylate), —COR (acyl), —CN, —SO₂R (sulfone), —SO₂NR₁R₂ (sulfamide), —P(O)(OR)₂.

### IV. Kits of the Invention

Any of the compositions described herein may be comprised in a kit. The kits will thus comprise, in suitable container means, an antimicrobial composition of the present invention.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the antimicrobial composition and any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred. The compositions may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit. However, the components of the kit may be provided as dried powder(s). When reagents and/or components are provided as a dry powder, the powder can be reconstituted by the addition of a suitable solvent. It is envisioned that the solvent may also be provided in another container means.

### EXAMPLES

The following examples are offered by way of example and are not intended to limit the scope of the invention in any manner.

### Example 1

### Exemplary DXR Inhibitor Development

### Exemplary Studies

### Background

Synthesis and QSAR studies of bisphosphonates as antiparasitic agents (Hudock et al., 2006; Kotsikorou et al., 2005) had been performed. A panel of 82 bisphosphonates were made and tested on two new targets, which are Trypanosoma brucei (causing sleeping sickness) vacuolar pyrophosphatase (Kotsikorou et al., 2005) and Trypanosoma cruzi (causing Chagas disease) hexokinase (Hudock et al., 2006). Several of these compounds showed potent activities against these two enzymes and they were active against these two parasites in vitro and in vivo. Good 3D-QSAR models (using comparative molecular field and comparative molecular similarity index, or CoMFA and CoMSIA, methods) were also successfully built, which are able to predict the experimental results well. In addition, active bisphosphonates showed no obvious toxicities against human cell lines as well as mice, indicating that development of such compounds is useful.

Synthesis and QSAR studies of bisphosphonates as anti-HIV agents (Song et al., 2008), which can restore the activity of AZT (3′-azido, 3′-deoxythymidine) on resistant HIV strains, had been performed. Computational design and synthesis of novel pyridinium bisphosphonates, targeting multiple proteins in isoprenoid biosynthesis pathways (Chen et al., 2008; Guo et al., 2007; Zhang et al., 2009) had been performed. These compounds are not only potent inhibitors of farnesyl diphosphate synthase, as conventional bisphosphonate drugs are, but they potently inhibit other longer prenyl diphosphate synthases, e.g., geranylgeranyl diphosphate synthase.

Phosphonosulfonates potently inhibit Staphylococcus aureus virulence (Liu et al., 2008; Song et al., 2008; Song et al., 2009). Continuing emergence of highly virulent, drug resistant S. aureus has prompted consideration of unconventional approaches to anti-infective therapy, including neutralization of specific bacterial virulence factors. One such virulence factor in S. aureus is the golden carotenoid pigment known as staphyloxanthin. The inventors have found similarities between the early enzymatic steps of staphyloxanthin production and the pathway for human cholesterol biosynthesis provide a new window for anti-infective development. Specifically, the two key enzymes in these two pathways, i.e., S. aureus dehydrosqualene synthase (CrtM) and human squalene synthase (SQS) were found to catalyze similar reactions. The inventors have solved the first crystal structures of CrtM (Liu et al., 2008) and found it resembles that of human SQS. In specific embodiments, known human SQS inhibitors inhibit S. aureus CrtM, and this has turn out to be the case (Liu et al., 2008). A small library of SQS inhibitors were made, and a phosphonosulfonate compound, BPH-652, was found for the first time to be a potent CrtM inhibitor with a Kᵢ value of ˜20 nM. It also effectively blocked staphyloxanthin biosynthesis in vitro (IC₅₀-100 nM), resulting in colorless S. aureus which had significantly enhanced susceptibility to reactive oxygen species (e.g., H₂O₂ and human blood) mediated killings. More importantly, BPH-652 also effectively protected mice from systemic staphylococcal infections, reducing ˜98% bacterial burden.

With this lead compound, the inventors carried out SAR/QSAR studies and found that 4′-substituted analogs of BPH-652 are selective CrtM inhibitors with essentially no activity against human SQS (Song et al., 2009). Next, they designed and found a phosphonoacetamide (which could be more bioavailable) is also a potent inhibitor of CrtM as well as staphyloxanthin biosynthesis (Song et al., 2009). Its crystal structure in CrtM was also solved to understand how it binds to the protein.

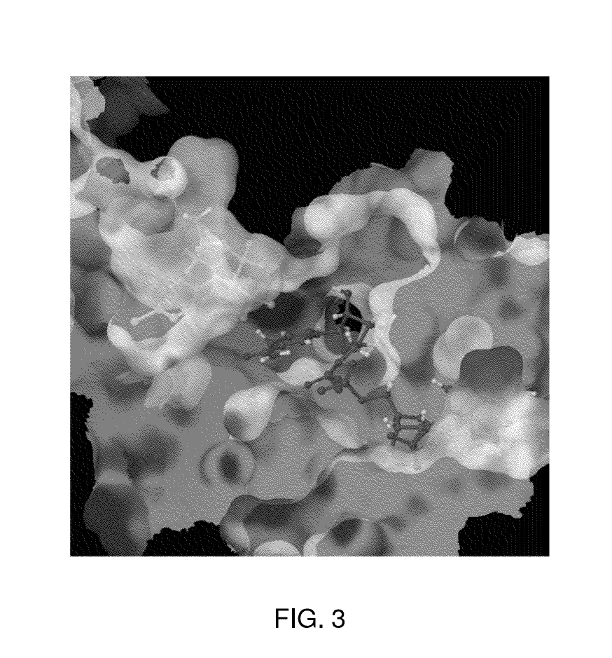

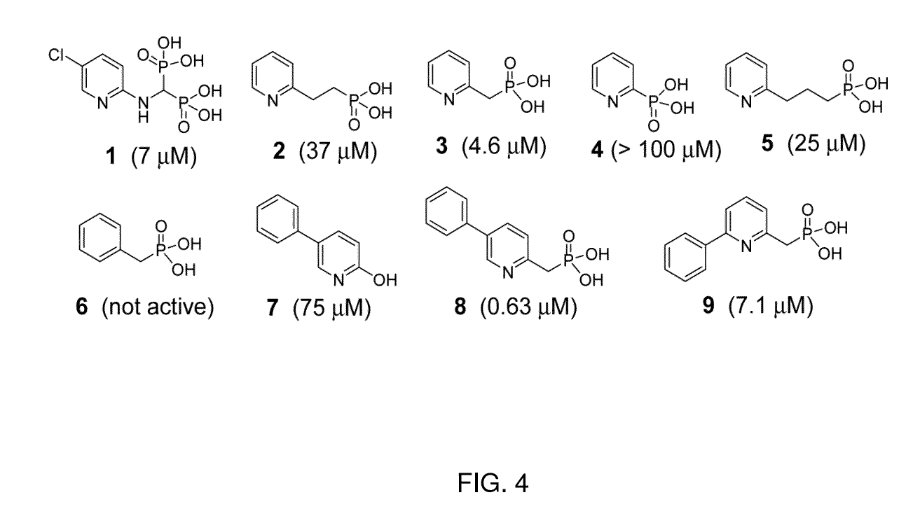

Structure based rational design leads to novel, potent DXR inhibitors. To complement the activity of fosmidomycin and overcome its limitations, in certain embodiments of the invention lipophilicity is an important feature for the design of a new generation DXR inhibitors, as it provides good cell membrane permeability, which is essential for killing bacteria and parasites. However, previous work on developing more lipophilic derivatives of fosmidomycin failed to yield potent inhibitors (Merckle et al., 2005; Ortmann et al., 2007). Nevertheless, from the crystal structures of DXR, it was possible for a hydrophobic molecule to inhibit DXR, although the substrate DXP and fosmidomycin are all highly polar molecules. As shown in FIG. 3, the substrate DXP, as well as fosmidomycin, only occupies a fraction of the active site. There is a large and mainly hydrophobic channel beside the substrate/fosmidomycin binding site, which connects the NADPH binding pocket to the exterior of the protein. In certain aspects of the invention this feature is exploited to design novel inhibitors that are more lipophilic (not fosmidomycin-like). In fact, a bisphosphonate compound 1 (shown in FIG. 4), which is also a DXR inhibitor (IC₅₀=7 μM) (43), is a good example. Also shown in FIG. 3, the bisphosphonate group binds to the Mg²⁺, with the lipophilic chloropyridine (in orange) extending to the hydrophobic side-channel. However, in certain cases one drawback for this inhibitor is that its bisphosphonate group is also extremely polar, which makes the whole molecule to have a logP value (octanol/water partition coefficient, calculated with QikProp in Schrödinger of −1.95. This shows that 1 is even more polar and water-soluble than fosmidomycin (logP: −1.69).

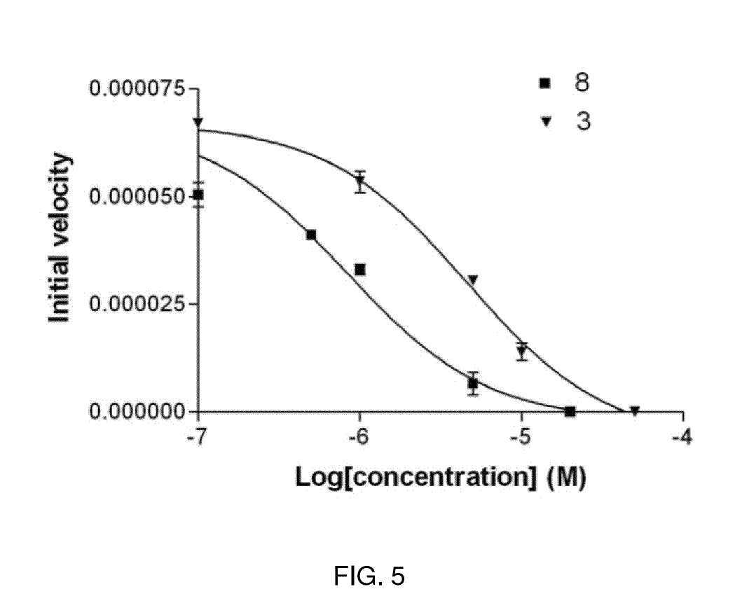

Upon further analysis of the binding mode of compound 1, the inventors found only one of its phosphonates bind to the Mg²⁺, with the other phosphonate group facing the NADPH binding pocket. This indicates that in some embodiments only one of the phosphonates is important and a mono-phosphonate compound, which is significantly more lipophilic, is also active, in some cases. The inventors thus made an mono-phosphonate compound 2, as shown in FIG. 4, and tested it against a recombinant E. coli DXR enzyme. Compound 2 was found to be also a DXR inhibitor with an IC₅₀ value of 37 μM. Although it is ˜5× weaker than 1, compound 2 has a significantly improved lipophilicity, with a calculated logP value of 0.88, predicted to be ˜670× more lipophilic than 1. The inventors then carried out a small-scale structure activity relationship (SAR) study based on this compound and identified compound 3, with one methylene shorter than 2, which is a good DXR inhibitor with an IC₅₀ value of 4.6 μM, being 150% and 8× more active than compounds 1 and 2, respectively. The dose response curve of 3 that was used to calculate the IC₅₀ value is shown in FIG. 5. Its calculated logP value is 0.58, predicted to have an improved lipophilicity (compared to fosmidomycin and 1). Compound 4 with a phosphonate directly linked to a pyridine ring, is a very weak inhibitor with an IC₅₀ value of >100 μM. Compound 5, which is one CH₂ longer than 2, has an IC₅₀ value of 25 μM. In addition, in certain embodiments it was found that the pyridine ring is very important for the activity, as compound 6 with a benzene ring instead, has essentially no activity.

On the other hand, a random screen of the inventors own compound library (containing ˜120 compounds at the time), resulted in the identification of compound 7 (FIG. 4) as a relatively weak DXR inhibitor with an IC₅₀ value of 75 μM. Interestingly, similar to 2-4, compound 7 (2-hydroxy-5-phenyl-pyridine) features a pyridine ring bearing a polar substituent at 2-position. The activity of 7, albeit weaker, does show that a hydrophobic compound (logP value of 7 is 2.45) can still inhibit DXR. In fact, given the Kₘ value of the substrate DXP is ˜100 μM (Koppisch et al., 2002; Kuzuyama et al., 2000), compound 7 binds to the protein even stronger than DXP. This also indicates that DXR can accommodate a bigger molecule having at least two aromatic rings. The inventors thus made compounds 8 and 9. Compound 8 was found to be indeed a potent DXR inhibitor with an IC₅₀ value of 0.63 μM, or a Kᵢ of 310 nM, as shown in FIG. 5. It should be noted that compound 8 is the first non-fosmidomycin-like inhibitor of DXR that has a submicromolar activity. Introduction of a phenyl group into the 5-position of compound 3 not only leads to a 7-fold activity increase, but also results in a logP value of 1.92. Compound 8 is thus predicted to be 22× more lipophilic than 3 and ˜4,000× than fosmidomycin. Compound 9 with a 6-phenyl is also a good DXR inhibitor, having an IC₅₀ value of 7.1 μM.

### Exemplary Research Design and Methods

The present invention utilizes traditional medicinal chemistry as well as computational, structure based drug design to develop novel small molecule inhibitors of 1-deoxy-D-xylulose-5-phosphate reductoisomerase (DXR), and they are tested on their in vitro biological activities against the growth of pathogenic bacteria and parasites. DXR is a validated target for the development of anti-infective drugs against a broad spectrum of pathogenic bacteria and protozoan parasites, such as P. aeruginosa, M. tuberculosis, P. falciparum and T. gondii. Fosmidomycin has been the only potent DXR inhibitor and has potent antibacterial and anti-malaria activity against certain pathogens. However, as a very polar molecule, it cannot penetrate into cells of many pathogenic species (e.g., M. tuberculosis). In addition, presumably also due to its hydrophilicity, it has a short half-life in plasma.

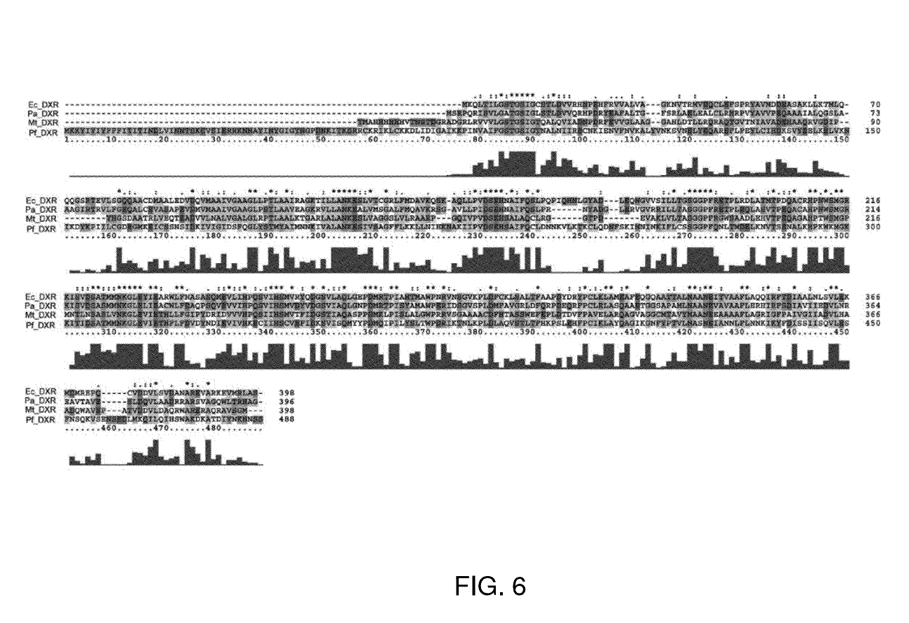

During the past few decades, drug resistant infective agents have become more and more predominant, which makes physicians short of weaponry to treat these infections. On the other hand, the pharmaceutical industry has produced fewer and fewer new anti-infective drugs during the same period, mainly because of a low investment return on this class of drugs. There is therefore an urgent need to find new anti-infectives that work on a distinct pathway from current drugs. A potent DXR inhibitor with a more lipophilic property is a useful compound for this purpose. Moreover, DXR is a particularly valued target, as it has been found to be highly conserved across species. FIG. 6 shows a ClustalX alignment of DXR enzymes from four different species, i.e., E. coli, P. aeruginosa, M. tuberculosis and P. falciparum, including three major human pathogens. The enzymes from the first three bacteria show a high degree of homology, with 32% identity and 63% similarity. Although P. falciparum DXR is larger and shows a little more difference, the overall degree of homology is still high, especially with respect to the residues at the active site. Thus, DXR inhibitors has broad activities, in certain embodiments of the invention. Indeed, fosmidomycin potently inhibit DXRs from all species cloned and expressed to date (Kuzuyama et al., 1998; Jomaa et al., 1999; Altincicek et al., 2000; Dhiman et al., 2005; Woo et al., 2006; Carretero-Paulet et al., 2002; Giessmann et al., 2008; Grolle, 2000; Mueller et al., 2000; Schwender et al., 1999; Rohdich et al., 2006).

The inventors have used structure based rational design and identified a novel class of potent, more lipophilic DXR inhibitors (e.g., compound 8). In certain aspects of the invention, other compounds are identified, and their activities are characterized using a variety of in vitro biological assays, for example.

### Rational Drug Design and Synthesis

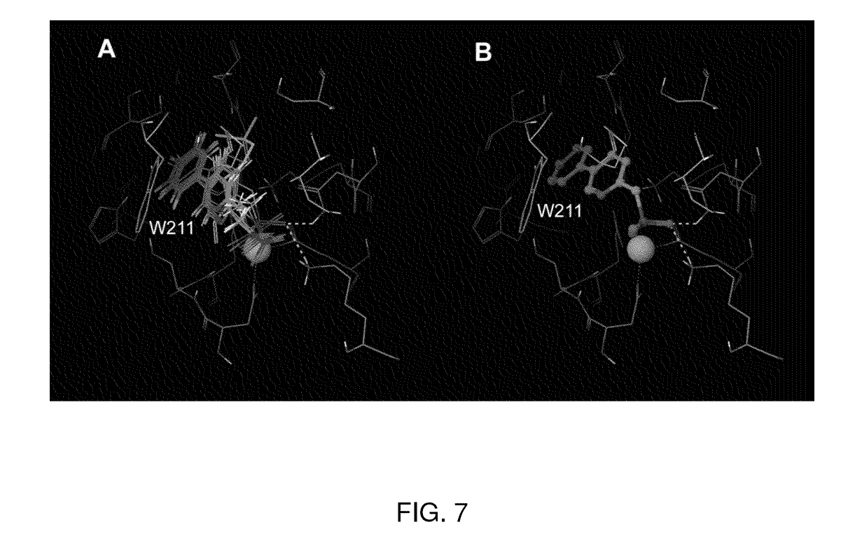

In certain embodiments, there is rational design and synthesis of potent DXR inhibitors based on the scaffold of the lead inhibitor 8 (or 3), for example. Structure-based computational methods may be employed to guide drug design. Since compound 8 is a potent DXR inhibitor but has a distinct structure from that of fosmidomycin, the binding mode of 8 is thus of interest and are useful to design compounds with improved activity. A reliable method is to solve the x-ray crystal structure of DXR-8 (or 3) complex. In addition, the inventors have performed a docking study, using Glide (Englebienne et al., 2007; Friesner et al., 2004; Kontoyianni et al., 2004; Perola et al., 2004) in Schrödinger suite 2008 (61). FIG. 7A shows 20 docking structures of compound 8 in DXR crystal structure (PDB code: 2egh), which are tightly clustered with each other, showing a good docking result. Their phosphonate groups all bind to the Mg²⁺ and have H-bonds and electrostatic interacts with protein sidechains. It is noteworthy that the phosphonate group of fosmidomycin does not bind to the Mg ion and is located in a different position, also superimposed in FIG. 7A. The hydrophobic phenyl and pyridine rings of 8 are located in a mainly hydrophobic pocket. It is remarkable that the pyridine ring seems to have a π-π interaction with Trp211, which is fully conserved across species (FIG. 6).

The docking result indicates that in certain aspects of the invention: 1) an aromatic ring, e.g., the pyridine rings in 3 and 8, is useful for the activity, as it interacts with the indole ring of a conserved residue Trp 211 through a π-π stacking (at least in some cases); 2) a magnesium-binding group that can interact with Mg²⁺ is also important; 3) a hydrophobic group enhances the activity. Based on this information together with the structure activity relationship (SAR) analysis of compound 3 analogs in FIG. 4, the inventors use medicinal chemistry to synthesize several series of analogs of compounds 8 and 3 and carry out a 3D-QSAR study of this class of compounds. The QSAR results and the x-ray crystal structure of DXR with the novel inhibitors (e.g., 3 or 8) facilitates drug design and development.

### Medicinal Chemistry Modifications of Compound 8 (or 3).

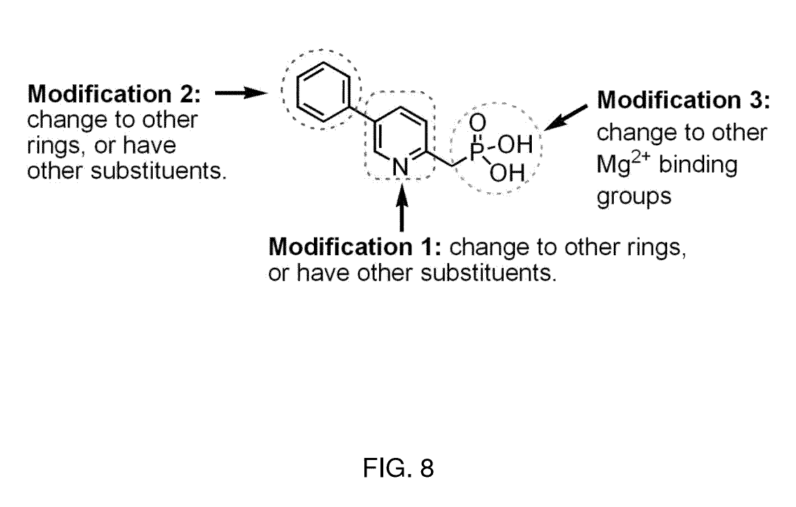

One embodiments for medicinal chemistry modifications of compound 8 is illustrated in FIG. 8.

Modification 1. Since the docking result indicates the usefulness of an aromatic ring, the following exemplary compounds are made to characterize the SAR of the ring:

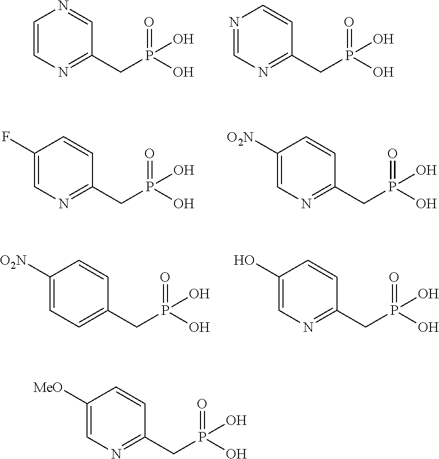

The first two compounds contain a pyrazine and a pyrimidine ring. The 3rd and 4th compounds have electron withdrawing groups —F and —NO₂. The pyridine rings of these compounds should be more electron-deficient. In addition, comparison of the activities of the 5th compound and compound 6 (FIG. 8) further demonstrate how an electron-withdrawing group (e.g., —NO₂) affects the activity. The oxygen containing groups in the last two compounds are expected to have a different property, as they can provide electrons to the pyridine ring.

Next, one can make the following compounds to optimize the substituent of the pyridine ring:

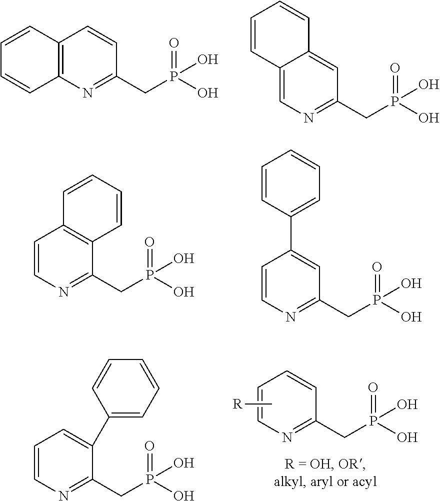

Activities of the first five compounds show which position are useful for introducing a hydrophobic group. Based on the last structure, one can make several series of compounds, with an alkyl, an alkoxyl, an aryl, or an acyl group, for example, to perform a detailed structure activity relationship study, from which the substituent of the pyridine ring may be optimized, in certain cases.

In general, methods for synthesizing this class of compounds are quite straightforward, involving attaching a phosphonate group to a pyridine ring. Two exemplary general methods to introduce the phosphonate group are shown below:

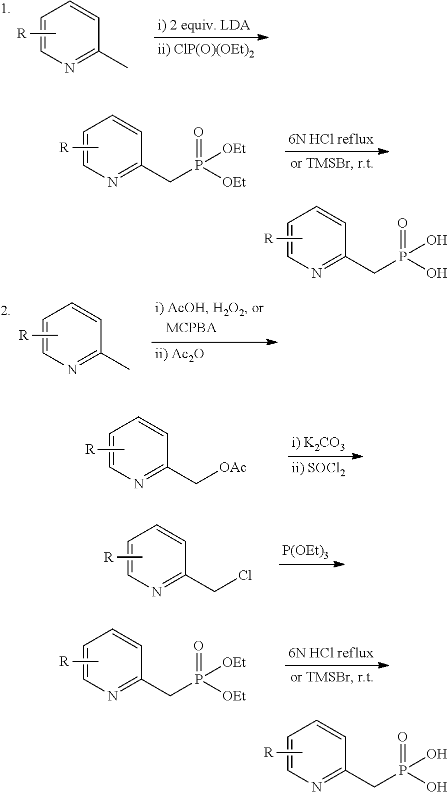

The first method uses lithium diisopropylamide (LDA) to generate an organolithium salt of picoline, which is then reacted with diethyl chlorophosphate to, after hydrolysis, give the target compound, phosphonomethylpyridine. For substituents that are sensitive to LDA (a very strong base), an alternative, milder method 2 are used. A picoline is treated with H₂O₂/AcOH or meta-chloroperbenzoic acid (MCPBA), followed by acetic anhydride, to give a picolinyl acetate. Upon hydrolysis and treatment with SOCl₂, the resulting picolinyl chloride is reacted with triethyl phosphite (Arbuzov reaction) to afford, after hydrolysis, the target compound.

Modification 2. This modification focuses on optimizing the substituent on the phenyl ring of compound 8 in order to obtain compounds with better activity. Basically, one can use the following reaction to generate a compound library:

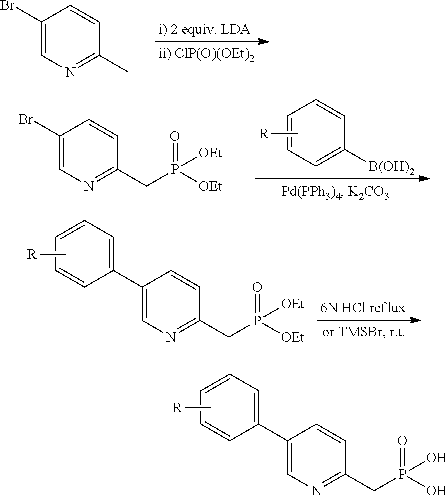

The main step for the introduction of a substituted phenyl to the pyridine is the well documented Suzuki coupling reaction, using a bromopyridine and a boronic acid (or ester) catalyzed by a palladium compound (e.g., Pd(PPh₃)₄). Since there are a large collection of commercially available boronic acids/esters, one can first select ˜30 to make a small compound library. Based on QSAR/SAR analysis of this type of compounds (described below) as well as others (e.g., compounds in Modifications 1 and 3), more compounds are designed and synthesized.

Modification 3. Although the exemplary lead inhibitor 8 has a greatly improved lipophilicity (compared to fosmidomycin), it still has a polar phosphonate group, which is negatively charged at the physiological pH. This feature may limit the oral and/or bio-availability, in at least some cases. This modification focuses on using other less polar, preferably neutral group to replace the phosphonate. In addition, an alternative way is a prodrug strategy, i.e., to use a neutral, hydrolysable group (esters or phosphamide) to mask the phosphonate. Upon entering into cells, the protection group is hydrolyzed chemically or by, e.g, an esterase or phosphamidase, to give the active DXR inhibitor.

Docking studies showed that the phosphonate group in 8 not only binds to the Mg²⁺, but also has two H-bonds with the protein (FIG. 4). One can make the following compounds and test their activity:

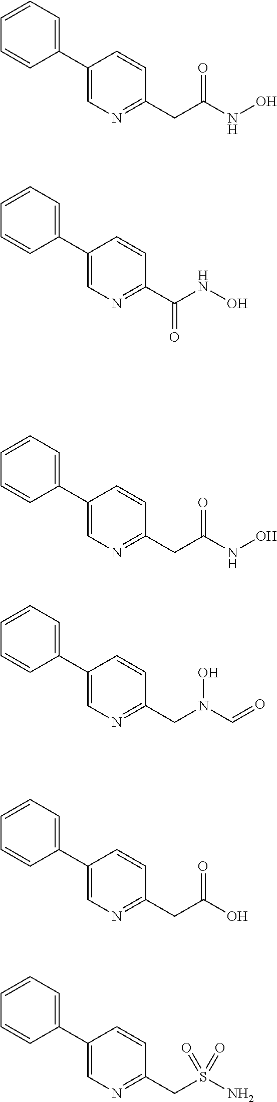 [SMILES: O=C(Cc1ccc(-c2ccccc2)cn1)NO ; O=C(NO)c1ccc(-c2ccccc2)cn1 ; O=C(Cc1ccc(-c2ccccc2)cn1)NO ; O=CN(O)Cc1ccc(-c2ccccc2)cn1 ; O=C(O)Cc1ccc(-c2ccccc2)cn1 ; NS(=O)(=O)Cc1ccc(-c2ccccc2)cn1]

Due to the potential dual actions of the phosphonate, a hydroxamate group in the first four compounds could be a good isostere, which can chelate the Mg²⁺ and is able to form hydrogen bonds with the protein. The first three compounds are all hydroxamates designed based on compounds 3 and 8. The fourth compound is a reserved hydroxamate, as found in fosmidomycin. The last two compounds contain a carboxylate and a sulfamide functional groups, which are also known to interact with metal ions. These phosphonate substitutions are less polar and most are actually neutral groups at the physiological pH. If any of them is identified to be a potent DXR inhibitor, one can make more analogs to further develop it. In addition, syntheses of these compounds are simple, mainly involving coupling of an acid with a hydroxylamine using a carbodiimide or a similar transformation, for example.

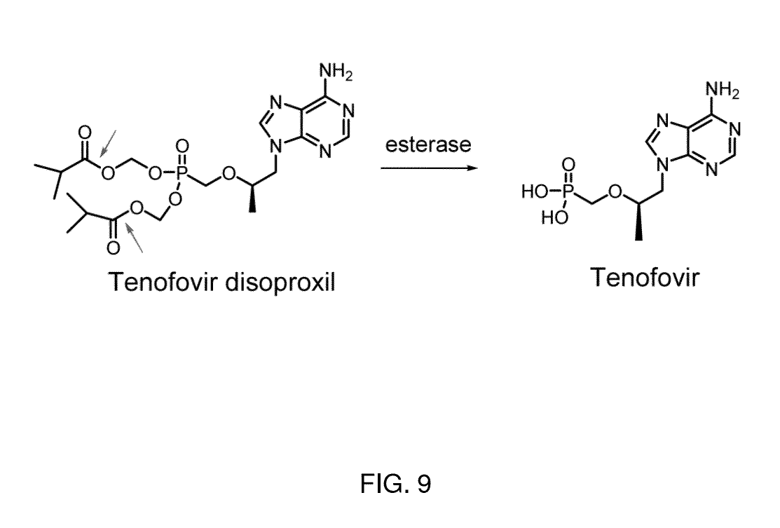

Next, one can use a prodrug strategy to mask the phosphonate group, which can make the inhibitors more lipophilic and enhance the oral availability. For example, this strategy has been successfully applied to develop nucleotide reserve transcriptase inhibitor antiviral drugs, such as tenofovir disoproxil, as shown in FIG. 9. There are a number of hydrolysable groups that have been used to mask phosphate/phosphonate group. One can first synthesize the following prodrugs of compound 3 or 8 and test their activity against the growth of bacteria and parasites. These compounds are tested in human cell growth assay to evaluate their potential toxicity. However, since these protecting groups, such as that found in tenofovir disoproxil, have already been on the market or tested in animals and/or humans, they are also not toxic in certain aspects.

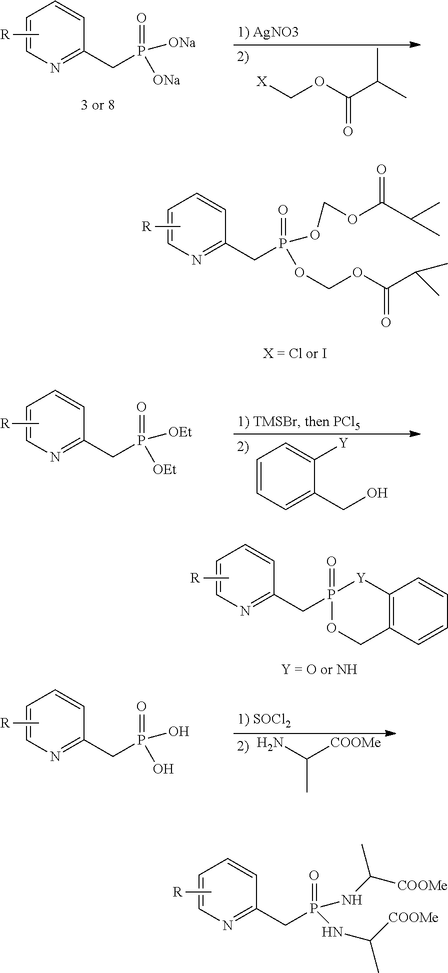

### QSAR and X-Ray Structural Determination

In order to rationally design compounds with improved activity against DXR as well as bacterium/parasite growths, in certain embodiments one can use two methods, i.e., QSAR and x-ray structural determination, to guide further drug design.

Quantitative structure activity relationship (QSAR) studies. After obtaining the enzyme and/or cell activity data of compounds synthesized as described elsewhere herein (with a total number of compounds >30 or more), one can carry out 3D-QSAR studies, using comparative molecular similarity index analysis (CoMSIA) (75) and comparative molecular field analysis (CoMFA) (Cramer et al., 1989) methods. Using these QSAR results, in which activity is related to 3D-structure in a quantitative manner, it is possible to optimize the electrostatic, steric, hydrophobic and H-donor/acceptor field requirements for DXR inhibition.

In some cases, a potent inhibitor (against an enzyme) does not always have good cell activity, as cell membrane permeability as well as other factors affect its effectiveness, for example. A typical example is recent phosphonosulfonate CrtM/staphyloxanthin biosynthesis inhibitors (Song et al., 2008). The inventors therefore first chose to use SlogP (the logarithm of the octanol/water partition coefficient) to describe this effect by using the following equation:

pIC₅₀(STX,cell)=a·pIC₅₀(CrtM)+b·S log P+c

where a, b and c are regression coefficients from a linear regression analysis. This yielded an R²=0.60 for the experimental-versus-predicted pIC₅₀ values or an R²=0.53 for a leave-two-out (L2O) prediction test set, which is a significant improvement. The inventors next used a three descriptor model:

pIC₅₀(STX,cell)=a·pIC₅₀(CrtM)+b·B+c·C+d

where B, C are all possible descriptor pairs available in a computational software MOE (MOE, 2006). The best model yielded R²=0.72, which is clearly an improvement over that obtained using solely CrtM or CrtM and SlogP results. The R² in this leave two out test set was R²=0.62, a major improvement over the R²=0.16 using solely enzyme inhibition data. Clearly, phosphonosulfonates are potent inhibitors of the CrtM enzyme, and their activity can be relatively well predicted by using the combinatorial descriptor search method, even when the cell/enzyme data is poorly correlated.

In certain embodiments of the invention, this method is employed to analyze the biological activity results, if enzyme activity is not well correlated with cellular activity (e.g., bacterial killing activity).

X-ray structural determination. Although the inventors have performed docking studies to predict how lead inhibitors (e.g., 8) bind to DXR, in certain cases an x-ray crystallographic study is carried out. Previous structural investigations revealed that DXR undergoes a series of conformational rearrangements (Mac Sweeney et al., 2005): apo-DXR has an “open” conformation; upon binding of NADPH, it shows a partially closed structure; when the substrate or an inhibitor is further complexed, it adopts a closed, tight binding mode. Moreover, a loop region consisting of residues 206-216 (including the fully reserved hydrophobic residue Trp211) acts as a flexible “lid” when the substrate or an inhibitor enters into the enzyme (Mac Sweeney et al., 2005; Yajima et al., 2002). The conformation of these residues has been found to be flexible. However, the docking results, using the crystal structure containing fosmidomycin as the ligand (Yajima et al., 2007), need further consideration. For example, the docking showed that there is a π-π interaction between the pyridine ring of 8 and the indole ring of Try211. However, previous x-ray structural study showed that the position of the Trp211 indole ring varies considerably in the presence or absence of fosmidomycin (Mac Sweeney et al., 2005). Since the structures of lead inhibitors are significantly different from that of fosmidomycin, crystal structures of DXR in complex with the compounds are useful.

### Biological Activity Testing of DXR Inhibitors.

In some embodiments of the invention, the biological activities of the DXR inhibitors synthesized are tested. For example, one can use a recombinant E. coli DXR enzyme as a primary screen. Active compounds are further tested on DXR enzymes from bacterium species M. tuberculosis and protozoan species P. falciparum and T. gondii. One can also test the activity of DXR inhibitors against the growth of a variety of microorganisms, in order to determine their in vitro anti-infective activity. Third, one can test the activity of DXR inhibitors against the growth of mammalian cells, in an effort to evaluate their potential toxicity.

### Enzyme Assays

One can use E. coli DXR as a primary screen. The protein expression and purification can be carried out as reported (Kuzuyama et al., 1998). E. coli M15 strain (Qiagen) is used as a host, in some cases. After transfection, the bacterium is cultured at 37° C. in LB medium containing kanamycin (25 μg/mL) and ampicillin (50 μg/mL). Upon reaching an optical density of 0.6 at 600 nm, DXR expression is induced with 0.2 mM isopropylthiogalactoside for 5 hours. Cells are harvested by centrifugation and resuspended in 100 mM Tris-HCl (pH 8.0). After brief sonication, the lysate is centrifuged at 10,000 g for 20 min and the supernatant was collected. A 50% slurry of Ni-NTA resin (Qiagen) is added into the supernatant and stirred on ice for 60 min. The resin is washed with 50 mM imidazole in 100 mM Tris-HCl (pH 8.0) and then the protein which binds to the Ni-NTA resin is eluted with 200 mM imidazole in 100 mM Tris-HCl (pH 8.0). The protein is stored in small aliquots at −80° C.

An E. coli DXR enzyme assay is utilized. The exemplary methodology is based on an initial linear consumption of NADPH and can be monitored by UV absorbance at 340 nm, where NADPH has the maximum absorption. DXP and NADPH are commercially available from Echelon and Sigma, respectively. Fosmidomycin may be used as a positive control in the assay. The assay is carried out in a 96-well microplate using 2 nM enzyme, 2 mM MgCl₂, 100 μM DXP (same as its Kₘ of ˜100 μM (Koppisch et al., 2002; Kuzuyama et al., 2000)), 50 μM NADPH in 50 mM HEPES buffer (pH=7.5) containing 0.1 mM DTT and 25 μg/mL BSA. For inhibition assays, compounds are pre-incubated with the enzyme for 20 min at 37° C., before initiation of the reaction by adding the substrate DXP. The decreasing absorbance at 340 nm of each well is monitored using a Beckman DTX-880 microplate reader. The initial velocities of wells containing increasing concentrations of an inhibitor are calculated and input into Prism (version 4.0). The IC₅₀ values as well as Kᵢs will be calculated by using standard dose response curve fitting in the software.

It should be noted that previous studies demonstrated that fosmidomycin can show either a competitive mode of inhibition (without pre-incubation) or a non-competitive inhibition (with pre-incubation) (Koppisch et al., 2002). The Kᵢs for these two modes were reported to vary significantly: Kᵢ (competitive)=215 nM versus Kᵢ (non-competitive)=21 nM. This is because fosmidomycin is a slow, tight-binding inhibitor, which binds and leaves the enzyme slowly. One can carry out a detailed enzyme kinetics assay to determine the inhibition modes of compounds 8 and 3, using variable concentrations of substrate DXP, NADPH and inhibitors, for example.

One can test the inhibitors against DXRs from P. falciparum, T. gondii and M. tuberculosis, for example. The enzyme activities of the inhibitors against DXRs from these pathogenic species is determined and useful.

Cell based assays. Next, one can test antibacterial activity of the DXR inhibitors against a panel of selected bacteria. Five bacterium species are chosen to be tested, which are E. coli (ATCC 25922), P. aeruginosa (ATCC 27853), Haemophilus influenzae (ATCC 10211), Bacillus cereus (ATCC 10987) and Bacillus subtilis (ATCC 82). They are all wild-type bacteria and can be purchased from American Type Culture Collection. The first three species are Gram negative and the last two are Gram positive bacteria, all of which only use the non-mevalonate pathway to produce essential IPP and DMAPP. Fosmidomycin was reported to have potent activity against the first three Gram negative bacteria (Mine et al., 1980; Neu and Kamimura, 1981), but is not active against Gram positive bacteria.

The minimum inhibition concentrations (MIC) of the inhibitors against these bacteria is determined, using a standard NCCLS (National Committee for Clinical Laboratory Standards) protocol. Bacterial inoculums re added to each well of a 96-well microplate containing 200 μL of serial inhibitor dilutions in LB or Mueller-Hinton broth. After incubation for 18-24 h at 35° C. with shaking, MIC is determined with a Beckman DTX-880 microplate reader at 540 nm as the lowest concentration of compound whose absorbance was comparable to the negative control wells. Fosmidomycin as well as three types of antibiotics, e.g., ampicillin, kanamycin and ciprofloxacin, are used as positive controls.

One can test the DXR inhibitors in this work against the in vitro growth of P. falciparum, T. gondii and M. tuberculosis. These species use the non-mevalonate pathway and their growth is inhibited by the compounds of the invention, in certain aspects of the invention.

Counterscreen using mammalian cells. In order to assess potential toxicity of the DXR inhibitors, one can test the activity of the inhibitors against the growth of mammalian cells (e.g., 3T3). 5,000 cells are inoculated into each well of a 96-well plate and cultured in RPMI-1640 medium supplemented with 10% fetal bovine serum at 37° C. in a 5% CO₂ atmosphere with 100% humidity overnight for cell attachment. After addition of compounds to each well, plates were then incubated for 2 days after which a standard MTT ((3-(4,5-dimethylthiazole-2-yl)-2,5-diphenyltetrazolium bromide) cell proliferation assay is used to obtain dose response curves. IC₅₀s of each compound are then calculated.

### Example 2

### A Coordination Chemistry Based Approach to Lipophilic Inhibitors of 1-deoxy-D-xylulose-5-phosphate reductoisomerase

Despite great success in the development of antibiotics, bacterial infections are still the number one cause of human mortality in the world, killing ˜6 million people each year. Furthermore, drug resistant bacteria have reached epidemic levels during the past few decades (Leeb, 2004). Even in developed countries, bacterial infections have now become a serious threat to public health, mainly because of rising drug resistance. On the other hand, production of new antibiotics by the pharmaceutical industry has decreased considerably since 1980 (Nathan, 2004). There is, therefore, a pressing need to find new drugs to combat bacterial infections that are resistant to current therapies.

1-Deoxy-D-xylulose-5-phosphate reductoisomerase (DXR) in the non-mevalonate isoprene biosynthesis pathway used by most bacteria as well as malaria parasites is an attractive target for developing novel anti-infective drugs (Hunter, 2007; Rodriguez-Concepcion, 2004; Testa and Brown, 2003), since humans do not have the enzyme. Fosmidomycin (1, Chart 1), a naturally occurring antibiotic (Mine et al., 1980; Neu and Kawimura, 1981) has been found to be a very potent DXR inhibitor (Kuzuyama et al., 1998).

Chart 1: Structures of 1-5, together with their IC₅₀ values (or % inhibition at 100 μM) against E. coli DXR in parenthesis.

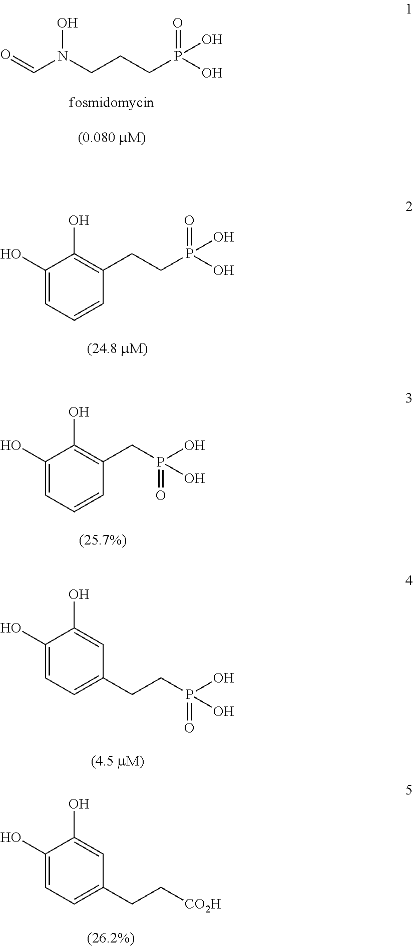 [SMILES: O=CN(O)CCCP(=O)(O)O ; O=P(O)(O)CCc1cccc(O)c1O ; O=P(O)(O)Cc1cccc(O)c1O ; O=P(O)(O)CCc1ccc(O)c(O)c1 ; O=C(O)CCc1ccc(O)c(O)c1]

It has also shown antimalarial activity in recent clinical trials (Borrmann et al., 2004; Borrmann et al., 2006). However, 1 has limited cellular uptake for many bacteria (Dhiman et al., 2005; Brown and Parish, 2008) as well as a short half-life (˜1 h) in plasma (Borrmann et al., 2004; Borrmann et al., 2006), due to its high hydrophilicity. Much interest has therefore been generated to develop potent DXR inhibitors and it has turned out to be a great challenge (Shtannikov et al., 2007; Kuntz et al., 2005; Merckle et al., 2005; Munos et al., 2008; Ortmann et al., 2007; Silber et al., 2005; Woo et al., 2006; Yajima et al., 2004; Gottlin et al., 2003). In this example, the discovery of novel, lipophilic DXR inhibitors with good antibacterial activity is discussed, using a coordination chemistry and structure based approach.

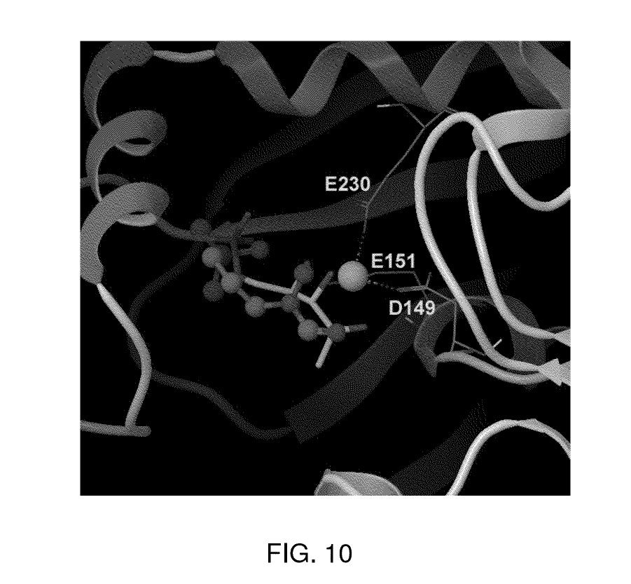

DXR is a Mg²⁺-dependent enzyme, catalyzing the isomerization and reduction of 1-deoxy-D-xylulose-5-phosphate (DXP) to 2-C-methyl-D-erythritol-4-phosphate with NADPH as a hydride donor. The crystal structure of the DXR-1 complex (Yajima et al., 2007) shows that the hydroxamate group of 1 chelates the central Mg²⁺ ion, which is anchored to the protein by coordination to the sidechains of the residues Asp149, Glu151 and 230 (FIG. 10). The substrate DXP binds to DXR in a similar manner (FIG. 10) (Mac Sweeney et al., 2005). These results suggested that coordination chemistry could be used to design a strong Mg²⁺-chelating group as a viable approach to DXR inhibition.

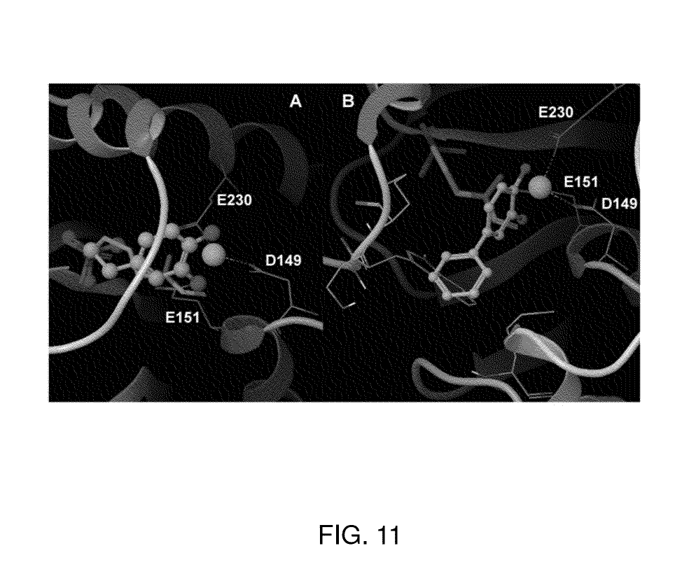

Mg²⁺ is a hard metal ion due to its small ionic radius, high electronegativity and low polarizability. It therefore only forms stable complexes with di-oxygen based, hard ligands, such as catechol and hydroxamate. In addition, the stability constant (k₁) of the Mg²⁺-catechol complex was found to be 1.7×10⁵ (Athavale et al., 1966), which is ˜3× as stable as Mg²⁺-hydroxamic acid complex (kᵢ=5.2×10⁴) (Agrawal and Roshania, 1982), suggesting catechol compounds, such as 2-4 that mimic the structure of 1, could be DXR inhibitors. Indeed, 2 was found to be an inhibitor of E. coli DXR with an IC₅₀ value of 24.8 μM. Although 3 had only 25.7% inhibitory activity at 100 μM against the enzyme, compound 4 was found to be a good DXR inhibitor with an IC₅₀ of 4.5 μM. A docking study using Glide (in Schrödinger 2008) showed that it could have a similar binding mode as 1, with catechol chelating the Mg²⁺ and the phosphonate located very close to that of 1, as shown in FIG. 11. In line with the result, the analogous compound 5, having a carboxylate group with less electrostatic interactions (compared to a phosphonate), exhibited only weak inhibition against the enzyme.

When 4 was tested against the growth of four bacteria that use the non-mevalonate pathway, i.e., E. coli, Pseudomonas aeruginosa, Bacillus anthracis and Micrococcus luteus, it exhibited, however, only weak antibacterial activity, with minimal inhibition concentrations (MIC, the lowest concentration of an agent that can inhibit visible bacterial growth upon 24 h incubation at 37° C.) being ≧1000 μM (Table 1). The negatively charged phosphonate group (at physiological pH) might limit the cell membrane permeability of 4, resulting in high MIC values.

TABLE 1
Minimal inhibition concentrations (μM) against 4 bacteria.

| | Gram-negative | | Gram-positive | |
|---|---|---|---|---|
| | bacteria | | bacteria | |
| compound | E. coli | P. aeruginosa | B. anthracis | M. luteus |
| 4 | >1000 | 1000 | 1000 | >1000 |
| 6 | 500 | 200 | 200 | 200 |
| 7 | 1000 | 200 | 200 | 200 |
| 8 | 100 | 50 | 20 | 100 |
| 1 | 10 | 20 | 100 | >1000 |
| ampicillin | 5 | 500 | 0.1 | 1 |
| kanamycin | 10 | 200 | 5 | 20 |

To design more lipophilic inhibitors that could have better cell permeability, we further looked into the DXR structure and found, beside the fosmidomycin/DXP and NADPH binding sites, there is a mostly hydrophobic pocket that can be exploited. Compounds 6 and 7 (Chart 2), which contain a catechol as a Mg²⁺ chelator and a simple lipophilic group (i.e., phenyl or benzyl) that could occupy the hydrophobic pocket, as suggested by the docking studies (FIG. 11B), were designed and synthesized. Both compounds were found to be modest DXR inhibitors with IC₅₀s of 44.7 and 22.4 μM. However, compared to 4, they exhibited better antibacterial activity (Table 1) with MICs of 200-1000 μM (or 37-190 μg/mL). Compounds 8-15, each of which has a different di-oxygen containing ligand as well as a phenyl/benzyl group, were made in an effort to identify a better Mg²⁺ chelating group for DXR inhibition. Although compounds 9-15 had no or weak activity, compound 8 containing a 1-hydroxypyridin-2-one group as a Mg²⁺ chelator was found to be a potent DXR inhibitor with an IC₅₀ value of 1.4 μM.

Chart 2. Structures of 6-18, together with their IC₅₀ values (or % inhibition at 100 μM) against E. coli DXR in parenthesis.

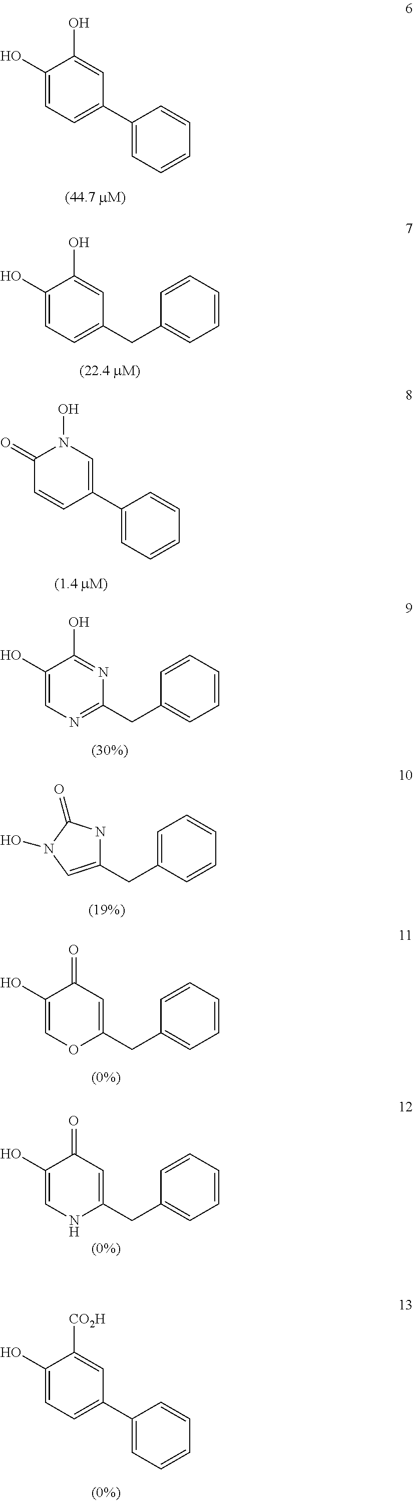

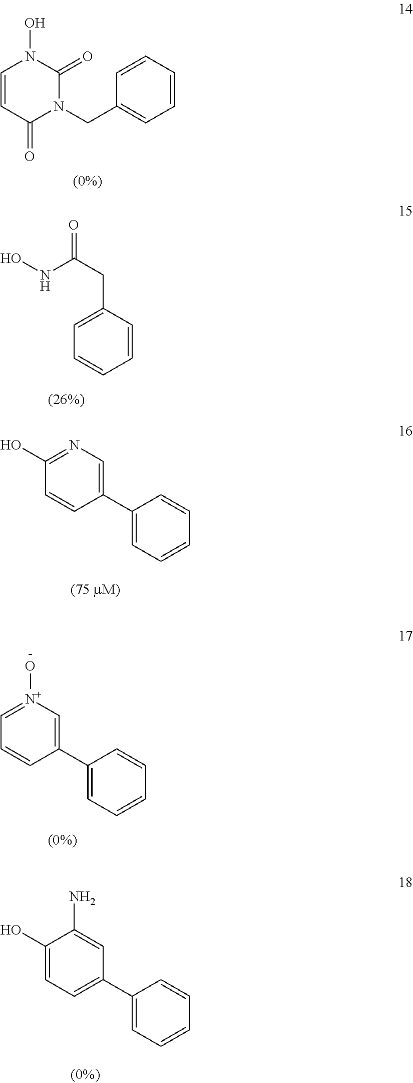 [SMILES: O=c1ccn(O)c(=O)n1Cc1ccccc1 ; O=C(Cc1ccccc1)NO ; Oc1ccc(-c2ccccc2)cn1 ; O[n+]1cccc(-c2ccccc2)c1 ; Nc1cc(-c2ccccc2)ccc1O]

Compound 8 exhibited good activity against all of the four bacterial species, with MICs of 20-100 μM (or 3.7-19 μg/mL) (Table 1). Three known antibiotics, i.e., 1, ampicillin and kanamycin, were included in the test as positive controls. Consistent with previous results,4 1 had potent activity against Gram-negative bacteria, but had weak or no activity against Gram-positive bacteria due to limited cellular uptake (Dhiman et al., 2005; Brown and Parish, 2008; Sakamoto et al., 2003). In addition, although 1 (IC₅₀=80 nM) is ˜16× more active than 8 against the E. coli DXR, 8 is more active against the growth of Gram-positive bacteria. This could be due to (1) increased lipophilicity and/or bioavailability of 8, (2) improved activity of 8 against DXRs from other bacteria, or both. It is also remarkable that 8 exhibited impressive antibacterial activity (MIC=50 μM or 9.4 μg/mL) against a clinical isolate of P. aeruginosa, which is a major hospital-acquired pathogen notorious for its significant intrinsic and acquired antibiotic resistance. This P. aeruginosa strain is highly resistant to two common antibiotics, ampicillin and kanamycin (MICs: 500 and 200 μM). However, it had a similar susceptibility to at least the novel DXR inhibitor 8, making it useful, including for further drug development.

To test if both —OHs (or tautomeric carbonyl) in 8 and 6 are involved in chelating the Mg²⁺, compounds 16-18 were prepared. Compared to 8, compounds 16 and 17 exhibited >50× weaker inhibition against DXR. In addition, the lack of activity of 18 also shows that —NH₂, a softer ligand, even with an adjacent, hard —OH, is not able to bind the Mg²⁺ strongly. These results clearly indicate that chelating the Mg²⁺ is key to DXR inhibition.

In summary, a coordination chemistry and structure based approach was used to find a novel, lipophilic DXR inhibitor with a broad spectrum of antibacterial activity. Moreover, it is noteworthy that the hydroxamate group seems to be considered as the ligand of choice for many metalloenzymes and it has been widely used to design their inhibitors. The weak activity of 15 shows this is not always the case. Using coordination chemistry to design a wide range of ligands targeting the central metal ion should be a viable means to find novel metalloenzyme inhibitors. However, this approach has not been widely explored (Jacobsen et al., 2007; Jacobsen et al., 2006). In addition, given the generally poor pharmacokinetics of hydroxamates (Borrmann et al., 2004; Borrmann et al., 2006; Wada et al., 2004; Sanderson et al., 2004) this approach is of particular importance with respect to drug discovery and development.

The following text provides exemplary methods employed in this example.

### Synthesis

Compounds 2-5 of this Example can be readily prepared according to Scheme S1:

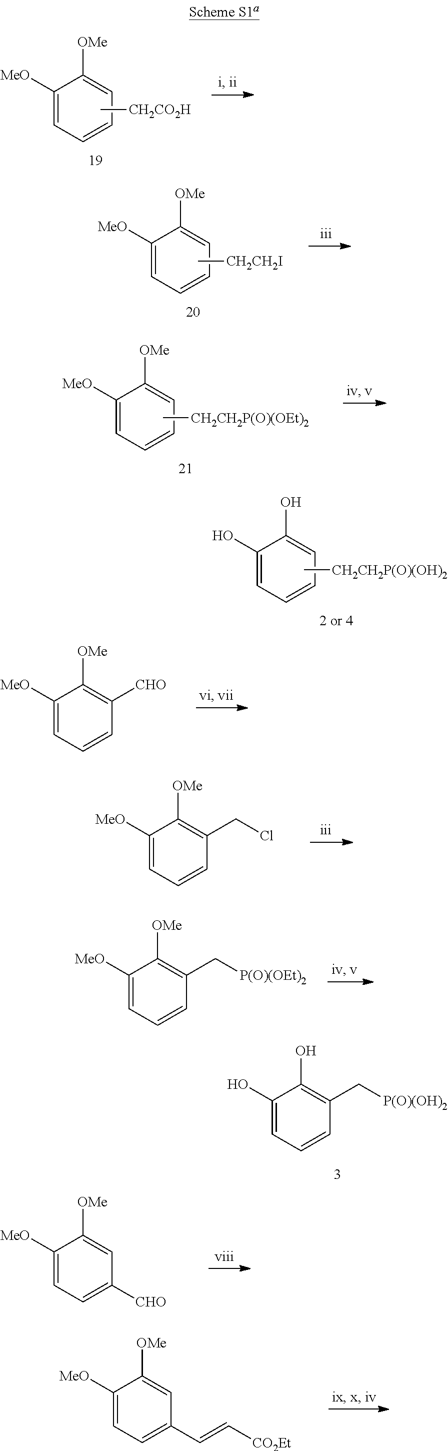

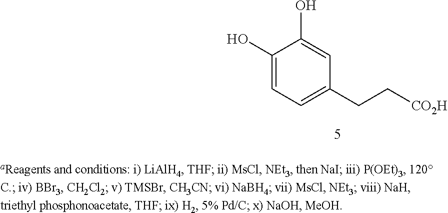 [SMILES: O=C(O)CCc1ccc(O)c(O)c1]

Dimethoxyphenylacetic acid 19 was reduced to the alcohol with LiAlH₄, which was converted to the corresponding iodide 20 by treatment with methanesulfonyl chloride followed by NaI. Arbuzov reaction using 20 and triethyl phosphite afforded phosphonate 21, which was then deprotected by successive treatments with BBr₃ and bromotrimethylsilane to give compound 2 or 4. Compound 3 was prepared similarly from 2,3-dimethoxybenzaldehyde. A Horner-Wadsworth-Emmons reaction using 3,4-dimethoxybenzaldehyde and sodium salt of triethyl phosphonoacetate, followed by hydrogenation and deprotections, gave compound 5.

Most compounds in Chart 2 were synthesized with a Suzuki coupling as the key step, according to Scheme S2:

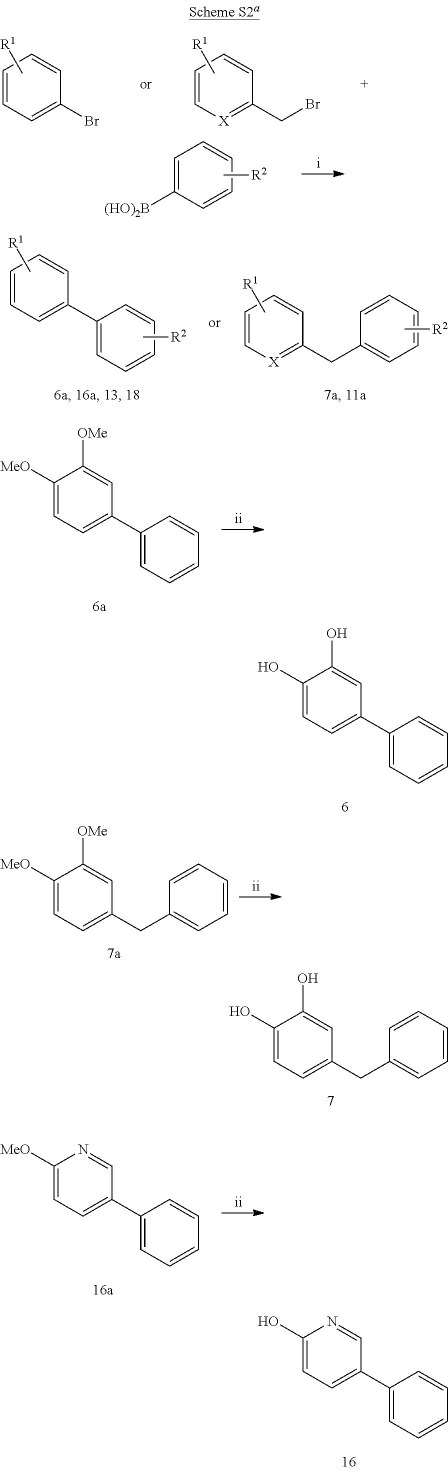

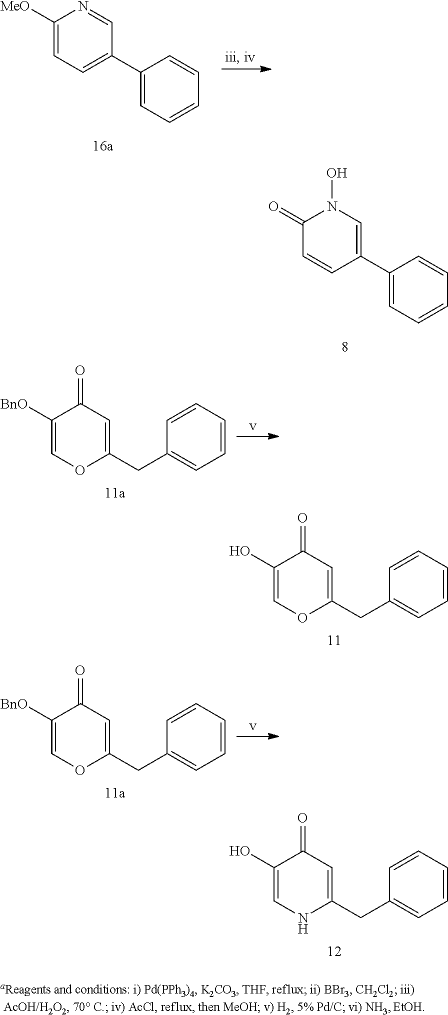 [SMILES: COc1ccc(-c2ccccc2)cn1 ; O=c1ccc(-c2ccccc2)cn1O ; O=c1cc(Cc2ccccc2)occ1OCc1ccccc1 ; O=c1cc(Cc2ccccc2)occ1O ; O=c1cc(Cc2ccccc2)occ1OCc1ccccc1 ; O=c1cc(Cc2ccccc2)[nH]cc1O]

Suzuki coupling using an appropriate bromide and a boronic acid gave, (if necessary) after a simple deprotection, compounds 6, 7, 11, 13, 16 and 18. Compound 8 was made from the intermediate 16a, upon an AcOH/H₂O₂ mediated oxidation and subsequent demethylation. Treatment of the intermediate 11a with ammonia in ethanol afforded, after hydrogenation, compound 12.

Compounds 9, 10, 14, 15 and 17 were made according to Scheme S3:

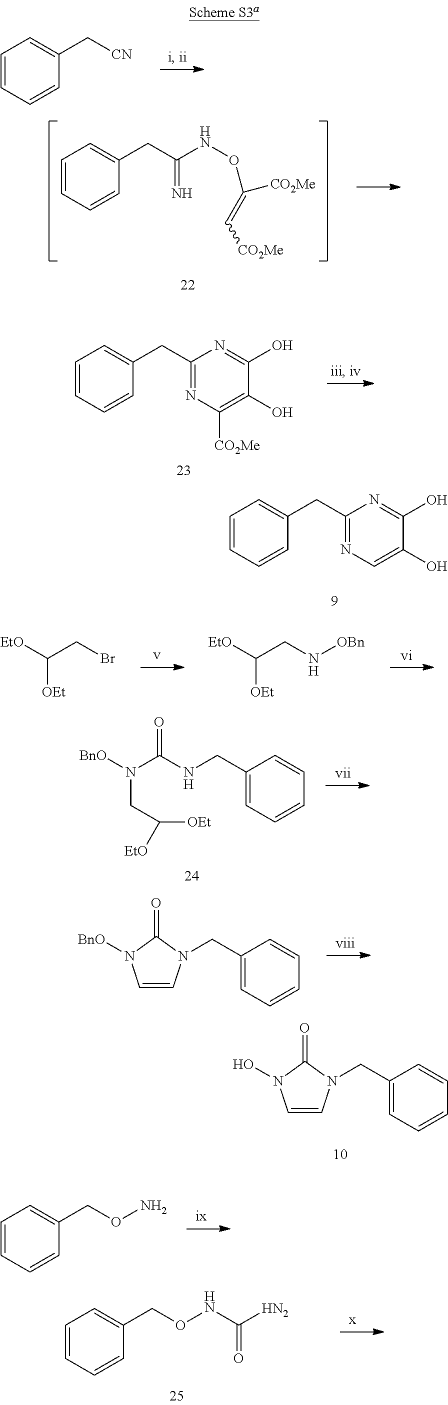

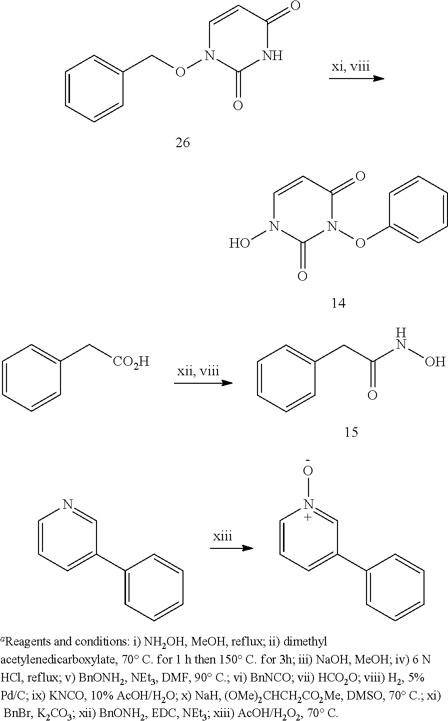

Compounds 9, 10 and 14 were prepared using published methods (Pace et al., 2007; Bock et al., 1987; Nguyen-Ba et al., 2000). In brief, benzyl cyanide was reacted with hydroxylamine and the resulting phenylacetyl amidoxime treated with dimethyl acetylenedicarboxylate to give compound 23. It was then hydrolyzed and decarboxylated to afford compound 9. O-Benzyl hydroxylamine was alkylated with 2-bromoacetaldehyde diethyl acetal, followed by treatment with benzylisocyanate, to produce urea 24. It was cyclized in the presence of 98% formic acid to give, after hydrogenation, compound 10. Urea 25, obtained from O-benzyl hydroxylamine and potassium cyanate, was reacted with methyl 3-dimethoxypropionate and NaH, affording compound 26, which was selectively alkylated and hydrogenated to give compound 14. Synthesis of compounds 15 and 17 were readily achieved according to standard protocols.

### Experimental Section

All reagents were purchased from Alfa Aesar (Ward Hill, Mass.) or Aldrich (Milwaukee, Wis.). The purities of all compounds were routinely monitored by using ¹H (at 400 MHz) and ³¹P NMR spectroscopy on a Varian (Palo Alto, Calif.) 400-MR spectrometer and, in some instance, absolute spin-count NMR quantitative analyses. High resolution mass spectra of new compounds with important biological activity were measure on a ThermoFisher LTQ-Orbitrap mass spectrometer.

General Method A: To a solution of a methoxybenzene (1 mmol) in dry CH₂Cl₂ (5 mL) was slowly added BBr3 (1.2 mmol per methoxy) at −20° C. Upon warming to room temperature, the reaction mixture was stirred overnight. 1N HCl (2 mL) was added to quench the reaction at 0° C. The product was extracted with ethyl acetate (3×10 mL). The combined organic phases were dried over NaSO₄, filtered and concentrated under reduced pressure. The product may be purified with a silica gel column chromatography.

General Method B: To a solution of a diethyl ester of phosphonic acid (1 mmol) in dry CH₃CN (3 mL) was added slowly bromotrimethylsilane (3 mmol) at 0° C. After stiffing overnight at room temperature, the solution was evaporated to dryness and MeOH (5 mL) was added to the residue. The solvent was removed under reduced pressure again and the residue redissolved in MeOH (5 mL). Neutralization with 2 M NaOH to pH=8 gave a sodium salt of a phosphonic acid as a white powder, which may be recrystallized in H₂O/acetone if needed. The purity can be calculated by using ¹H absolute spin-count quantitative analysis.

General Method C: A mixture of a bromide (1 mmol), a boronic acid (1.2 mmol), Na₂CO₃ (318 mg, 3 mmol) and Pd(Ph₃P)₄ (58 mg, 5 mmol %) in toluene/H₂O (v/v 5:3, 8 mL) or dry THF (if benzylic bromide is used) was heated to 90° C. under N₂ overnight. The product was extracted with EtOAc (3×10 mL) and purified with a silica gel column chromatography.

3-(2-Phosphonoethyl)catechol disodium salt (2). To 2,3-dimethoxyphenylacetic acid (364 mg, 2 mmol) in THF (10 mL) was slowly added LiAlH₄ (114 mg, 3 mmol) at 0° C. After stiffing for 2 h at room temperature, a few drops of water was used to quench the reaction and the reaction mixture filtered and washed with EtOAc. The filtrate was dried over Na₂SO₄, filtered and evaporated to dryness to give the alcohol, which was dissolved in CH₂Cl₂ (10 mL) and NEt₃ (0.3 mL, 2.4 mmol). Methanesulfonyl chloride (150 μL, 2 mmol) was added slowly at 0° C. After 1 h stiffing at room temperature, the reaction mixture was treated with water and extracted with CH₂Cl₂ (3×10 mL). The combined organic layers were washed with 1 N HCl and saturated aqueous NaHCO₃, dried, filtered and evaporated. The resulting residue was treated with NaI (0.9 g, 6 mmol) in acetone (7 mL) at 60° C. for 3 h. The solvent was removed, and then ethyl acetate (50 mL) and water (10 mL) were added. The organic layer was separated and washed with 5% Na₂S₂O₃, dried, filtered and evaporated to dryness to give the iodide 20. The crude iodide was heated with triethyl phosphite (1.04 mL, 6 mmol) at 120° C. under N₂ overnight. Upon removal of excess phosphite under reduced pressure, the residue was purified with column chromatography (silica gel, ethyl acetate) to give phosphonate 21, which was deprotected, following general methods A and B, to give compound 2 as an off-white powder (241 mg, 46% overall yield). ¹H NMR (400 MHz, D₂O): δ 6.68-6.62 (m, 3H), 2.66 (td, J=4.4, 8 Hz, 2H), 1.68-1.61 (m, 2H). ³¹P NMR (162 MHz, D₂O): δ 24.99.

3-(Phosphonomethyl)catechol disodium salt (3). To a solution of 2,3-dimethoxyphenylaldehyde (1.46 g, 10 mmol) in methanol (20 mL) was slowly added NaBH₄ (0.55 g, 15 mmol) at 0° C. After 1 h at room temperature, the solvent was removed under reduced pressure and the reaction was quenched with water and extracted with EtOAc (3×10 mL). The combined organic phases were dried over NaSO₄, filtered and concentrated under reduced pressure. The resulting crude alcohol was dissolved in CH₂Cl₂ (20 mL) and NEt₃ (2.8 mL, 20 mmol). Methanesulfonyl chloride (0.93 mL, 12 mmol) was added slowly at 0° C. After 1 h stiffing at room temperature, the reaction mixture was treated with water and extracted with CH₂Cl₂ (3×15 mL). The combined organic layers were washed with 1 N HCl and saturated aqueous NaHCO₃, dried, filtered and evaporated. The crude chloride (211 mg) was heated with triethyl phosphite (520 μL, 3 mmol) at 120° C. under N₂ overnight. Upon removal excess phosphite under reduced pressure, the residue was purified with column chromatography (silica gel, ethyl acetate) to give the corresponding phosphonate, which was deprotected, following general methods A and B, to give compound 3 as an off-white powder (155 mg, 68% overall yield). ¹H NMR (400 MHz, D₂O): δ 6.65-6.57 (m, 3H), 2.99 (d, J=21.2 Hz, 2H). ³¹P NMR (162 MHz, D₂O): δ 26.74.

4-(2-Phosphonoethyl)catechol disodium salt (4). Compound 4 was similarly prepared as described for compound 2 from 3,4-dimethoxyphenylacetic acid as an off-white powder (267 mg, 51% overall yield). ¹H NMR (400 MHz, D₂O): δ 6.68 (d, J=8.0 Hz, 1H), 6.66 (s, 1H), 6.57 (d, J=8.0 Hz, 1H), 2.58-2.449 (m, 2H), 1.64-1.558 (m, 2H). ³¹P NMR (162 MHz, D₂O): δ 25.12.

3-(3,4-Dihydroxyphenyl)propionic acid (5). To a solution of triethyl phosphonoacetate (1.1 mL, 5.5 mmol) in THF (17 mL) was added NaH (242 mg, 6.1 mmol, 60% in oil) at 0° C. 3,4-dimethoxybenzaldehyde (730 mg, 5 mmol) was added to the resulting solution. After being stirred for 2 h at room temperature, the reaction mixture was quenched with saturated NH₄Cl and extracted with EtOAc (3×15 mL). The combined organic phases were dried over NaSO₄, filtered and concentrated under reduced pressure. The crude product was hydrogenated with 5% Pd/C in methanol (15 mL) for 2 h to afford the desired saturated ester, which was dissolved in MeOH/H₂O (v/v 5:1, 12 mL). KOH (1.12 g, 20 mmol) was added in portion and then the reaction was stirred for 3 h. The solvent was removed under reduced pressure, and the resulting solid was dissolved in water, acidified with 6 M HCl, and extracted with EtOAc (4×10 mL). The combined organic phases were dried over NaSO₄, filtered and concentrated under reduced pressure. The residue was purified with column chromatography (silica gel, ethyl acetate/hexanes=1:1) to give the corresponding acid as a white solid, which was deprotected, following general method A, to give compound 5 as a white powder (770 mg, 81% overall yield). ¹H NMR (400 MHz, acetone-d6): δ 6.67-6.65 (m, 2H), 6.50 (dd, J=3.2, 5.6 Hz, 1H), 2.70 (t, J=8.0 Hz, 2H), 2.47 (t, J=8.0 Hz, 2H).

4-Phenylcatechol (6). General method C using 3,4-dimethoxyphenylbromide (217 mg, 1 mmol) and benzeneboronic acid (147 mg, 1.2 mmol) to give, after deprotection following general method A, compound 6 as a white powder (154 mg, 83%). ¹H NMR (400 MHz, CDCl₃): δ 7.53-7.50 (m, 2H), 7.40 (t, J=7.2 Hz, 2H), 7.32-7.27 (m, 1H), 7.12 (d, J=2.0 Hz, 1H), 7.06 (dd, J=2.0, 8.0 Hz, 1H), 6.94-9.92 (m, 1H), 5.13 (s, br, 1H), 5.09 (s, br, 1H).

4-Benzylcatechol (7). General method C using 3,4-dimethoxybenzyl chloride (135 mg, 0.81 mmol) and benzeneboronic acid (119 mg, 0.97 mmol) to give, after deprotection following general method A, compound 6 as a white powder (154 mg, 83%). 1H NMR (400 MHz, CDCl₃): δ 7.30-7.25 (m, 2H), 7.22-7.16 (m, 3H), 6.76 (d, J=8.0 Hz, 1H), 6.67-6.62 (m, 2H), 5.41 (s, br, 1H), 5.38 (s, br, 1H), 3.85 (s, 2H).

1-Hydroxy-5-phenylpyridin-2-one (8). General method C using 5-bromo-2-methoxypryridine (752 mg, 4 mmol) and benzeneboronic acid (586 mg, 4.8 mmol) to give compound 16a. It was dissolved in 8 mL acetic acid and hydrogen peroxide (1.45 mL, 30 mmol, 35% w/w) was then added. After refluxing for 18 h, the reaction mixture was allowed to cool, concentrated in vacuo, and extracted with ethyl acetate. The organic layer was dried over sodium sulfate and evaporated in vacuo. The residue was purified by a column chromatography (silica gel, 5% methanol in ethyl acetate) to get a white solid. It was then refluxed in acetyl chloride (5 mL) for 1 h, after which excess acetyl chloride was removed in vacuo. The residue was dissolved in methanol and stirred overnight at room temperature. Upon removal of solvent, the residue was washed with diethyl ether to get the pure product as an off-white powder (470 mg, 59% overall yield). ¹H NMR (400 MHz, CDCl₃): δ 8.02 (s, 1H), 7.67 (d, J=9.2 Hz, 1H) 7.46-7.43 (m, 5H), 6.82 (d, J=9.6 Hz, 1H); ¹³C NMR (100 MHz, CDCl₃): δ 156.9, 137.4, 135.5, 129.2, 129.0, 127.8, 126.1, 121.0, 117.5; HRMS (ESI) [M+H]⁺ Calcd for C₁₁H₁₀NO₂⁺: 188.0706. Found: 188.0710.

4,5-Dihydroxy-2-benzylpyrimidine (9) (Pace et al., 2007). A mixture of benzyl cyanide (585 mg, 5 mmol) and hydroxylamine (198 mg, 6 mmol) in MeOH (15 mL) was refluxed for 24 h. The solvent was removed under reduced pressure and the residue triturated with diethyl ether to give a white powder, which was reacted with equal molar amount of dimethyl acetylenedicarboxylate in CHCl₃ for 1 h at 70° C. After removal of the solvent, the residue was refluxed in xylene (10 mL) for additional 3 h. Upon cooling to room temperature, the precipitate was collected and washed with diethyl ether to give compound 23. It was dissolved in MeOH (30 mL) and treated with 3 equiv. NaOH (2 M in H₂O) overnight. The off-white precipitate was collected by filtration and washed with diethyl ether, which was then refluxed in 6N HCl (5 mL) for 1 h. Upon removal of the solvent, compound 9 was obtained by recrysallization from EtOH/ether as a white powder (246 mg, 26% overall yield). ¹H NMR (400 MHz, D₂O): δ 7.33 (s, 1H), 7.28-7.17 (m, 5H), 4.07 (s, 2H).

1-Hydroxy-3-benzylimidazol-2-one (10) (Bock et al., 1987). A mixture of O-benzyl hydroxylamine (370 mg, 3 mmol), 2-bromoacetaldehyde diethyl acetal (590 mg, 3 mmol), NEt₃ (420 μL, 3 mmol) in DMF (5 mL) was stirred at 90° C. for 24 h. Water (20 mL) and ethyl acetate (50 mL) were added and the organic layer separated, dried, evaporated and purified with a column chromatography (silica gel, ethyl acetate/hexane:1/5) to give a oil, which was reacted with equal amount of benzyl isocyanate in CH₂Cl₂ to give compound 24 quantitatively. It was treated with formic acid (5 mL) at room temperature overnight to give, after a column chromatography purification (silica gel, ethyl acetate/hexane:1/1) and hydrogenation (5% Pd/C, MeOH, 1 h), compound 10 as a white powder (305 mg, 53% overall yield). ¹H NMR (400 MHz, CDCl₃): δ 7.30-7.17 (m, 5H), 6.54 (d, J=3.2 Hz, 1H), 6.01 (d, J=3.2 Hz, 1H), 4.78 (s, 2H).

2-Benzyl-5-hydroxy-4H-pyran-4-one (11). General method C using 2-bromomethyl-5-benzyloxy-4H-pyran-4-one (Yamamoto et al., 1989) (885 mg, 3 mmol) and benzeneboronic acid (440 mg, 3.6 mmol) to give compound 11a, which was purified with a column chromatography (silica gel, ethyl acetate/hexane:1/1) and hydrogenated (5% Pd/C, MeOH, 1 h) to give compound 11 as a white powder (205 mg, 40% overall yield). ¹H NMR (400 MHz, CDCl₃): δ 7.77 (s, 1H), 7.35-7.25 (m, 3H), 7.22-7.20 (m, 2H), 6.24 (s, 1H), 3.84 (s, 2H).

2-Benzyl-5-hydroxypyridin-4(1H)-one (12). To a solution of 11a (292 mg, μmol) in ethanol/water (1/1, 6 mL) was added ammonium hydroxide (0.67 mL, 10 mmol). The mixture was refluxed at 70° C. for 15 h. Upon removal of the solvents, the residue was recrystallized with cold ethanol/water mixture to give, after hydrogenation (5% Pd/C, MeOH, 1 h), compound 12 as a white solid (115 mg, 60% yield). ¹H NMR (400 MHz, CDCl₃): δ 7.99 (s, 1H), 7.34-7.29 (m, 3H), 7.25-7.20 (m, 2H), 6.48 (s, 1H), 3.91 (s, 2H).

2-Hydroxy-5-phenylbenzoic acid (13). General method C using 5-bromo-2-hydroxylbenzonic acid (868 mg, 4 mmol) and benzeneboronic acid (586 mg, 4.8 mmol) to give compound 13 as a white powder (702 mg, 82%). ¹H NMR (400 MHz, D₂O): δ 10.48 (s, br, 1H), 8.13 (d, J=2.4 Hz, 1H), 7.76 (dd, J=2.4, 8.8 Hz, 1H), 7.56 (s, 1H), 7.55 (s, 1H), 7.43 (t, J=7.2 Hz, 2H), 7.36-7.31 (m, 1H), 7.08 (d, J=8.8 Hz, 1H).

1-Hydroxy-3-benzylpyrimidin-2,4-dione (14) (Nguyen-Ba et al., 2000). To a solution of benzyloxy urea (Nguyen-Ba et al., 2000) (332 mg, 2 mmol) in DMSO (4 mL) was added sodium hydride (120 mg, 3 mmol, 60% w/w) followed by ethyl 3,3-diethoxy-2-methylpropionate (0.279 mL, 2 mmol). The reaction mixture was stirred at 65° C. under nitrogen for 24 h after which the solvent was evaporated and the residue was purified with a column chromatography (silica gel, ethyl acetate) to give 26 as a white solid. It was then treated with benzyl bromide (1 equiv.) and potassium carbonate (345 mg, 2.5 mmol) in DMF (5 mL) for 18 h at room temperature. Water (20 mL) and ethyl acetate (50 mL) were added and the organic layer was separated, dried, evaporated, and purified by a column chromatography (silica gel, ethyl acetate:hexane 1:3). The product was hydrogenated (5% Pd/C, MeOH, 1 h) to give compound 14 as a white solid (181 mg, 60% overall yield). ¹H NMR (400 MHz, CDCl₃): δ 9.20 (br s, 1H), 7.46 (d, J=8.0 Hz, 1H,), 7.45-7.35 (m, 2H), 7.25-7.23 (m, 3H), 5.72 (d, J=8.0 Hz, 1H,), 5.06 (s, 2H).

Phenylacetohydroxamic acid (15). To a mixture of phenylacetic acid (136 mg, 1 mmol), BnONH2 (185 mg, 1.5 mmol) and NEt₃ (140 μL, μmol) in CH₂Cl₂ (8 mL) was added N-ethyl-N′-(3-dimethylaminopropyl) carbodiimide hydrochloride (286 mg, 1.5 mmol). After being stirred for 4 h, the reaction was quenched with 2 M HCl and extracted with CH₂Cl₂ (3×10 mL). The combined organic phases were washed with 1 M NaOH, dried over NaSO₄, filtered and concentrated under reduced pressure. The residue was purified with column chromatography (silica gel, ethyl acetate/hexanes=1:2) to give a colorless oil, which was hydrogenated with 5% Pd/C in methanol (5 mL) for 2 h to afford compound 15 as a white solid (135 mg, 90%). ¹H NMR (400 MHz, D₂O): δ 7.29-7.18 (m, 5H), 3.28 (s, 2H).

2-Hydroxy-5-phenylpyridine (16). General method C using 5-bromo-2-methoxypryridine (752 mg, 4 mmol) and benzeneboronic acid (586 mg, 4.8 mmol) to give, after deprotection following general methods A, compound 16 as a white powder (466 mg, 80%). ¹H NMR (400 MHz, CDCl₃): δ 7.80 (bs, 1H), 7.63 (bs, 1H), 7.46-7.38 (m, 5H), 7.34 (bs, 1H), 6.70 (bs, 1H).

3-Phenyl-pyridine-N-oxide (17). To the solution of 3-phenyl pyridine (210 mg, 2 mmol) in acetic acid (6 mL) was added hydrogen peroxide (0.97 mL, 20 mmol). After refluxing for 18 h, the reaction mixture was allowed to cool, concentrated, and extracted with ethyl acetate. The organic layer was dried, evaporated, and purified with a column chromatography (silica gel, 5% methanol in ethyl acetate) to give compound 17 as a white solid (190 mg, 82% yield). ¹H NMR (400 MHz, CDCl₃): δ 8.49 (s, 1H), 8.22 (d, J=6.0 Hz, 1H,), 7.55-7.44 (m, 6H), 7.36 (t, J=6.8 Hz, 2H).

2-Amino-4-phenylphenol (18). General method C using 2-amino-4-bromophenol (376 mg, 2 mmol) and benzeneboronic acid (366 mg, 3 mmol) to give product as a light brown solid (220 mg, 59%). ¹H NMR (400 MHz, CHCl₃): δ 8.26-8.22 (m, 4H), 7.63-7.57 (m, 2H), 7.54-7.48 (m, 4H).

DXR inhibition assay. E. coli DXR expression and purification were carried out as reported.5 In brief, E. coli M15 strain (Qiagen) was used as a host. After transformation, the bacterium was cultured at 37° C. in LB medium containing kanamycin (25 μg/mL) and ampicillin (50 μg/mL). Upon reaching an optical density of 0.6 at 600 nm, DXR expression was induced with 0.2 mM isopropylthiogalactoside for 5 hours. Cells were harvested by centrifugation and resuspended in 100 mM Tris-HCl (pH 8.0). After brief sonication, the lysate was centrifuged at 10,000 g for 20 min and the supernatant was collected and subjected to an affinity column chromatography using Ni-NTA resin (Qiagen). The resin was washed with 50 mM imidazole in 100 mM Tris-HCl (pH 8.0) and then the protein was eluted with 200 mM imidazole in 100 mM Tris-HCl (pH 8.0). The protein was stored in small aliquots at −80° C.

DXP (Meyer et al., 2004) and fosmidomycin (Kurz et al., 2006) were made according to published methods and NADPH was purchased from EMD (Gibbstown, N.J.). The enzyme assay was carried out in a 96-well microplate using 2 nM enzyme, 2 mM MgCl₂, 100 [μM DXP, 50 μM NADPH in 50 mM HEPES buffer (pH=7.5) containing 25 μg/mL BSA. For inhibition assay, compounds were pre-incubated with the enzyme for 20 min at 37° C., before initiation of the reaction by adding DXP. The decreasing absorbance at 340 nm of each well was monitored using a Beckman DTX-880 microplate reader. The initial velocities of wells containing increasing concentrations of an inhibitor were calculated and input into Prism (version 3.0, GraphPad Software, Inc., La Jolla, Calif.). The IC₅₀ values were calculated by using a standard dose response curve fitting in the software. The reported IC₅₀s were the mean values of at least two independent experiments.

Antibacterial activity assay. E. coli (B/r strain), P. aeruginosa (H45006 strain), B. anthracis (Sterne strain) and M. luteus (WT strain) were obtained from Dr. Adam Kuspa (Baylor College of Medicine), among which the P. aeruginosa strain was isolated from a patient's sputum (by Dr. James Versalovic, Baylor College of Medicine and Texas Children Hospital). The minimum inhibition concentrations (MIC) of each compound against these bacteria were determined, using a standard NCCLS (National Committee for Clinical Laboratory Standards) protocol. An overnight culture of each bacterium was diluted 50-fold into LB broth medium and incubated to an OD of 0.4 at 600 nm. The culture was then diluted 10.000-fold into LB broth medium and 10 μL of the inoculum was added into each well of a 96-well flat bottom microplate containing 190 μL of serial compound dilutions (0.1 μM ˜1000 μM) in LB broth. Upon incubation for 24 h at 37° C. with shaking, MIC was determined with a Beckman DTX-880 microplate reader at 600 nm as the lowest concentration of a compound whose absorbance was comparable to the negative control wells. The reported MICs were the mean values of at least two independent experiments. Ampicillin and kanamycin purchased from Sigma (St. Louis, Mo.) were used as positive controls.

### Example 3

### Crystallographic Studies of 1-deoxy-D-xylulose-5-phosphate Reductoisomerase Using Lipophilic Phosphonate Probes

### Role of TRP211 in Inhibitor Recognition

In the present Example, pyridine/quinoline containing phosphonates are identified to be DXR inhibitors with IC₅₀ values being as low as 840 nM. Three DXR:inhibitor structures are also provided, revealing a novel binding mode. The indole group of Trp211 is found to move ˜4.6 Å to open up a hydrophobic pocket, where the pyridine/quinoline rings of the inhibitors are located and have strong π-π stacking/charge-transfer interactions with the indole ring. Docking studies demonstrate the structures are useful to predict the binding modes of other lipophilic DXR inhibitors, which can be supported by their structure activity relationships. Overall, this work shows an important role of Trp211 in inhibitor recognition and provides a structural basis for drug design and development.

In certain embodiments of the invention, there is provided potent, more lipophilic inhibitors that might possess improved pharmacokinetic properties. Medicinal chemistry based on the structure of 1 has been investigated for the past decade (Haemers et al., 2006; Devrueux et al., 2007; Shtannikov et al., 2007; Kuntz et al., 2005; Merckle et al., 2005; Munos et al., 2008; Ortmann et al., 2007; Silber et al., 2005; Woo et al., 2006; Kurz et al., 2006). Although most of these modifications yielded compounds with considerably reduced activity, derivatives of 1 and 2 bearing an α-substituent, such as compounds 3-8 shown in Chart 1, are able to largely maintain the inhibitory activity (Haemers et al., 2006). The best in this series is compound 8 with a 3,4-dichlorophenyl group, which showed similar enzyme activity as 1. However, 8 demonstrated >10× increased activity against P. falciparum (EC₅₀ of 90 nM vs. 1.1 μM for 1), presumably due to enhanced cellular uptake resulted from its lipophilicity. Other lipophilic DXR inhibitors 3-7 also showed better antimalaria activity (EC₅₀s of 0.25-0.95 μM) than 1, despite their weaker enzyme activities. It is of interest that structure activity relationship (SAR) analysis of this series of compounds revealed that DXR inhibition appears to be correlated to the increased electron-deficiency of the phenyl ring (from 3 to 7,4-OMe<4-Me<H<4-Cl≈3,4-diCl). However, the underlying mechanism for the phenomenon remains unknown. In addition, DXR inhibitors that are structurally distinct from 1 are scarce. Two bisphosphonates 9 and 10 with a hydrophobic pyridine and isoquinoline sidechain were found to be relatively weak inhibitors (IC₅₀=4 and 7 μM) (yajima et al., 2004). A coordination chemistry based design was employed to find a lipophilic, drug-like DXR inhibitor 11 (IC₅₀=1.4 μM), which exhibits good activity against a broad spectrum of pathogenic bacteria (Deng et al., 2009).

Chart 1. Structures of known DXR inhibitors 1-11, together with their IC₅₀ values against E. coli DXR in parenthesis. (IC₅₀ values are from the original refs 11, 21 and 22)

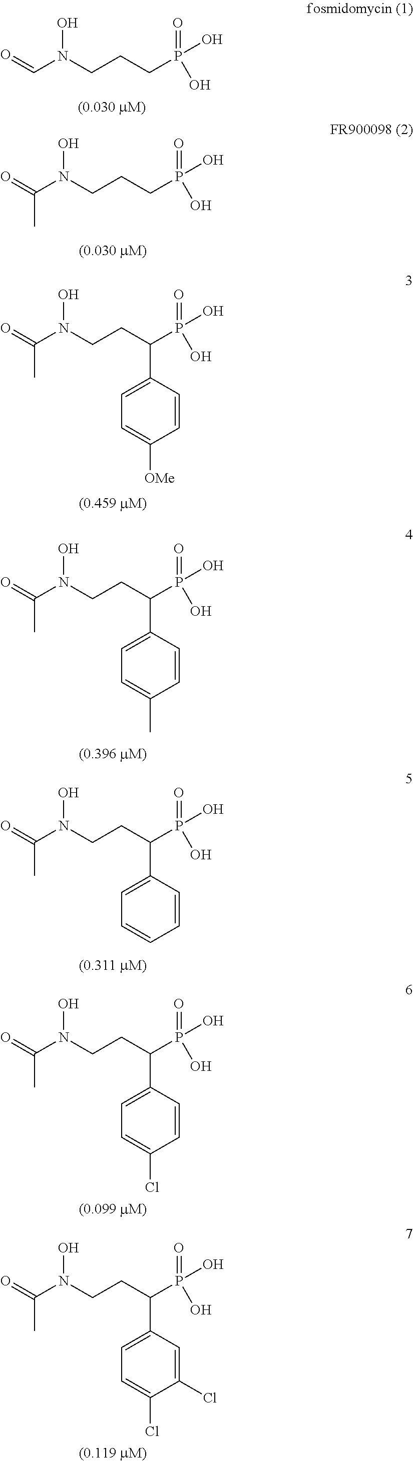

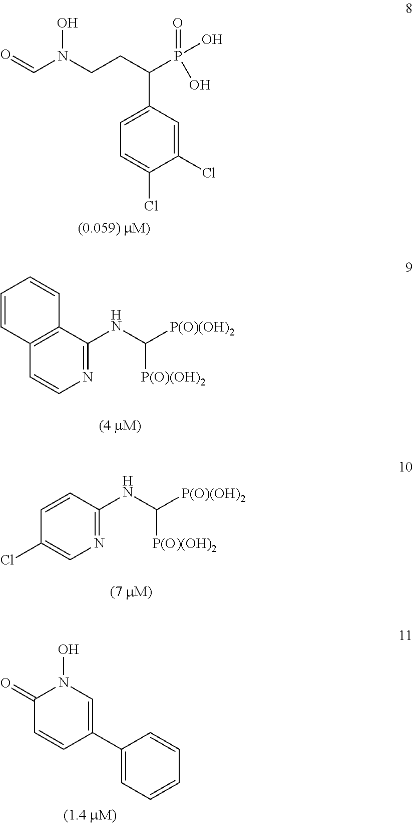

Since several lipophilic DXR inhibitors, particularly compound 8, possess potent enzyme activity as well as superior anti-infective activity, it is of interest to investigate how they bind to the protein, especially with respect to the binding site of their hydrophobic groups. The structural probing should facilitate further inhibitor design and development for antibacterial and antimalaria purposes, since these widespread, drug-resistant pathogens kill millions of people each year worldwide. However, x-ray structure of DXR in complex with 8 (or its analogs) is not available. In fact, to date, only two types of DXR:inhibitor structures have been reported, i.e., DXR with 123-26 and bisphosphonates 9 and 10 (Yajima et al., 2004).

Chart 2. Structures of 12-19, together with their IC₅₀ values against E. coli DXR in parenthesis.

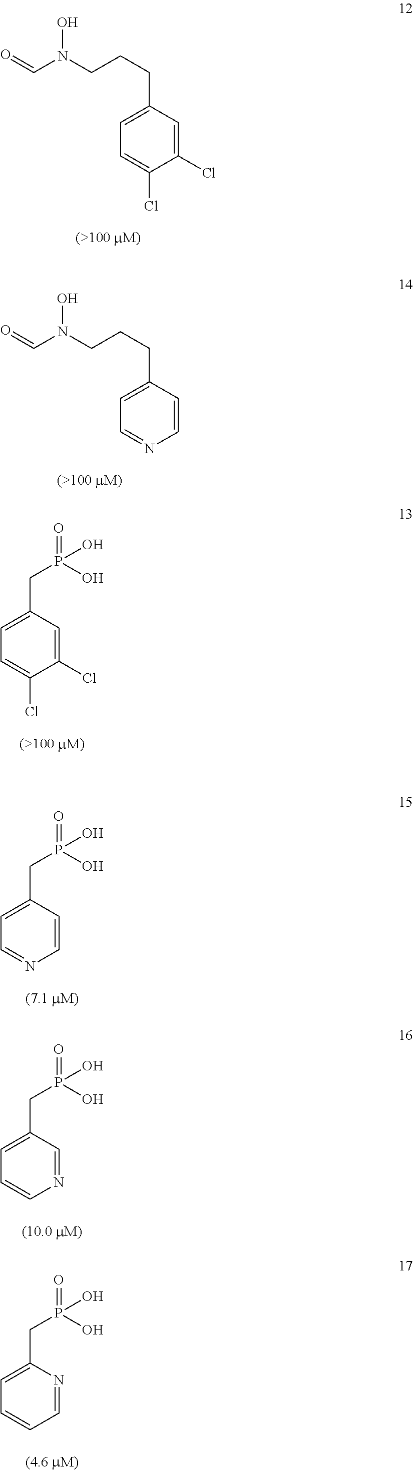 [SMILES: O=CN(O)CCCc1ccc(Cl)c(Cl)c1 ; O=CN(O)CCCc1ccncc1 ; O=P(O)(O)Cc1ccc(Cl)c(Cl)c1 ; O=P(O)(O)Cc1ccncc1 ; O=P(O)(O)Cc1cccnc1 ; O=P(O)(O)Cc1ccccn1]

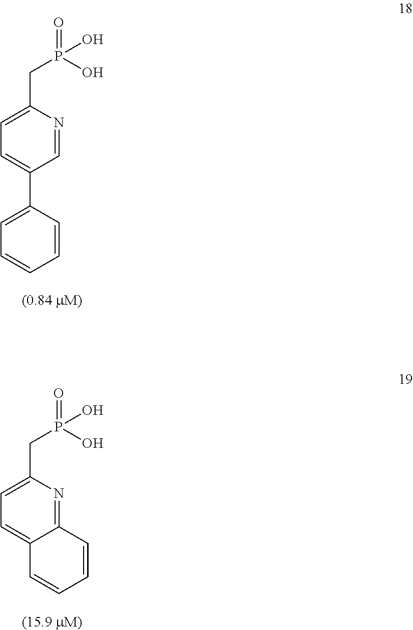 [SMILES: O=P(O)(O)Cc1ccc(-c2ccccc2)cn1 ; O=P(O)(O)Cc1ccc2ccccc2n1]

To find lipophilic analogs of 8 that are active against DXR, the inventors synthesized compounds 12 and 13 (Chart 2). These two compounds were designed by simply removing either the phosphonate group (for 12) or the hydroxamate moiety (for 13) from 8. However, when tested against recombinant E. coli DXR, both compounds were found to have essentially no inhibitory activity at 100 μM. Since SAR suggested a more electron-deficient ring could be favored,11 14 and 15 with a pyridin-4-yl group were prepared. Although hydroxamate 14 is also not active, compound 15, pyridin-4-ylmethylphosphonic acid, was found to be a DXR inhibitor with an IC₅₀ value of 7.1 μM. Analogous 16 and 17 with a pyridin-3-yl and -2-yl sidechain were subsequently made and found to be also active, with 17 having an IC₅₀ value of 4.6 μM. To further probe the hydrophobic binding site of DXR, more lipophilic compounds 18 and 19 with two rings were synthesized. 18, 5-phenylpyridin-2-ylmethylphosphonic acid, exhibits the strongest DXR inhibition in this series with an IC₅₀ value of 0.84 μM. Phosphonate 19 having a quinolin-2-yl group is weaker than 17 (IC₅₀=15.9 μM). Compounds 15-19 represent a new class of lipophilic DXR inhibitors that are structurally distinct from 1, with 18 being among the very few DXR inhibitors possessing submicromolar activity. These pyridine/quinoline phosphonates should be a new scaffold for further inhibitor development. Moreover, since these compounds have a big lipophilic group, they can be exploited to probe the elusive hydrophobic pocket(s) of DXR.

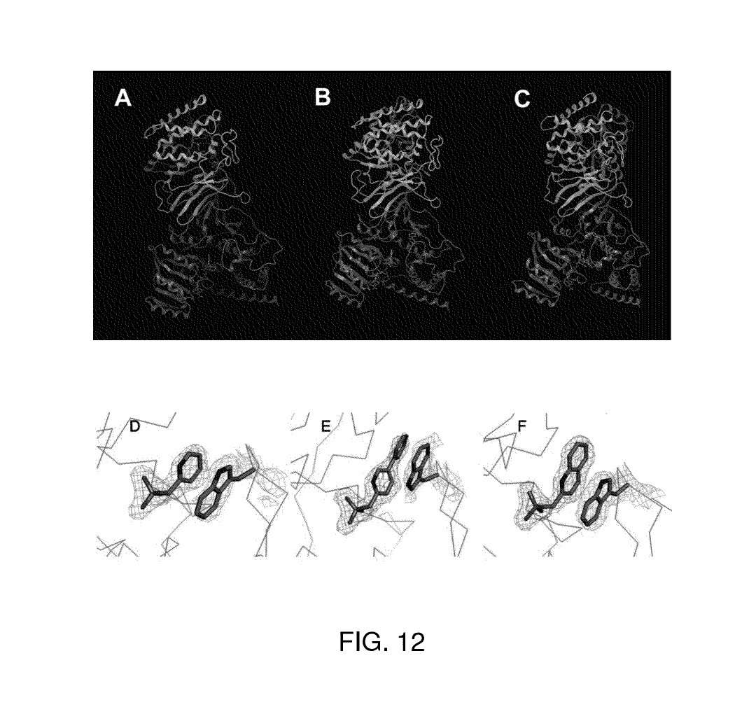

The inventors performed x-ray crystallographic studies of these lipophilic phosphonate inhibitors and obtained the crystal structures of compounds 17-19 complexed with E. coli DXR. The structures were refined at 2.1 Å (for DXR:17) or 2.0 Å (for DXR:18 and DXR:19) and the electron density of these inhibitors able to be obtained clearly. Full crystallographic details are shown in Table 2 and the overall structures of the DXR:17, DXR:18 and DXR:19 complexes in FIG. 12. All of the three protein complexes crystallize as homodimers, with each subunit containing one inhibitor and one NADPH molecule. In all cases, the overall 3-D protein structure is very similar to those previously reported (yajima et al., 2004; Yajima et al., 2002; Steinbacher et al., 2003; Mac Sweeney et al., 2005; Yajima et al., 2007) 17-19 bind to the same site as 1 and DXP, showing a competitive mode of action. The binding pose of NADPH is almost identical to those found in other DXR structures.

TABLE 2
Data collection and refinement statistics

| Crystal | DXR: 17 | | DXR: 18 | | DXR: 19 | |
|---|---|---|---|---|---|---|
| Data collection | | | | | | |
| Beamline | PF-NW12A | | | | | |
| Wavelength (Å) | 1.000 | | | | | |
| Resolution range (Å) | 50.0-2.1 | (2.14-2.1) | 50.0-2.0 | (2.03-2.0) | 50.0-2.0 | (2.03-2.0) |
| Space group | P2₁2₁2 | | | | | |
| Unit cell a/b/c (Å) | 181.7/59.3/87.2 | | 182.3/59.2/87.0 | | 182.6/59.3/87.2 | |
| Completeness (%) | 99.5 | (99.3) | 99.4 | (98.5) | 99.0 | (81.0) |
| I/□ | 21.8 | (5.0) | 25.4 | (4.4) | 29.0 | (4.8) |
| Redundancy | 3.5 | (3.4) | 3.7 | (3.6) | 3.7 | (3.5) |
| R_{merge}^{a} | 0.081 | (0.362) | 0.063 | (0.364) | 0.050 | (0.297) |
| Number of unique | 55758 | (2739) | 64256 | (3161) | 65518 | (2628) |
| reflections | | | | | | |
| Refinement | | | | | | |
| Resolution range (Å) | 50.0-2.1 | (2.15-2.1) | 50.0-2.0 | (2.052-2.0) | 50.0-2.0 | (2.052-2.0) |
| Number of reflection | 52715 | (2822) | 60935 | (3235) | 61556 | (3290) |

| | | | |
|---|---|---|---|
| R/R_{free}^{b} (%) | 18.4/22.5 | 19.2/21.7 | 18.4/21.9 |
| Number of atoms | | | |
| Protein | 6063 | 6063 | 6063 |
| Solvent | 413 | 381 | 411 |
| RMSD from ideality | | | |
| Bond length (Å) | 0.018 | 0.017 | 0.016 |
| Bond angle (°) | 1.804 | 1.621 | 1.725 |
| Average B factor (Å²) | | | |
| Protein | 22.3 | 23.1 | 24.1 |
| Solvent | 30.0 | 30.5 | 31.3 |
| Inhibitor | 17.2 | 14.7 | 18.5 |

^{a}R_{merge} = ΣₕΣᵢ|Iᵢ(h) − <I(h)>|ΣₕΣᵢIᵢ(h), where Iᵢ(h) is the ith measurement.
^{b}A subset of the data (5%) was excluded from the refinement and used to calculate R_{free}.

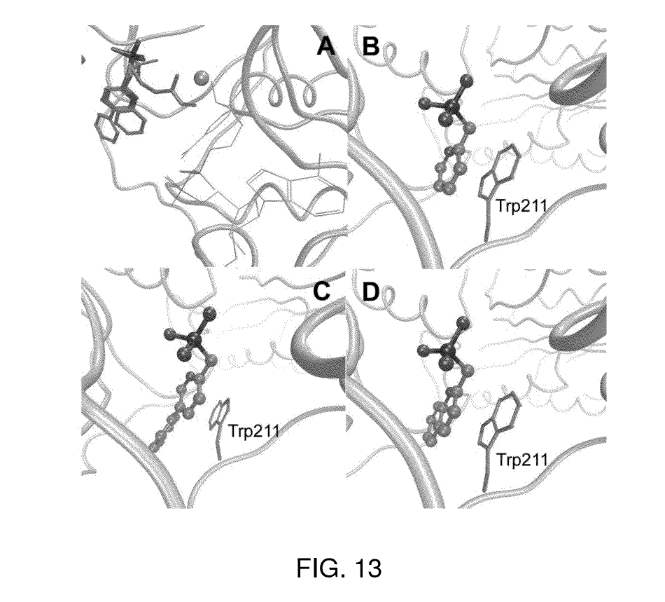

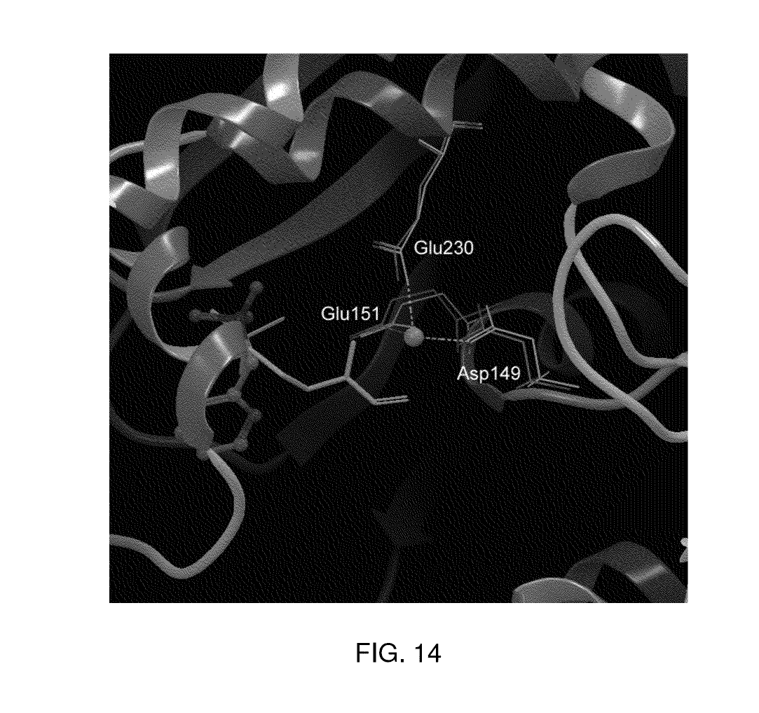

FIG. 13 shows the close-up views of the three DXR complexes. The phosphonate groups of the inhibitors are located in the phosphonate binding site of 1, having H-bonds and electrostatic interactions with the residues Lys227, Ser185 and Ser221. The three phosphonate groups almost overlap with each other (FIG. 13a). The distances between the P atoms of 17-19 and that of 1 are ˜1.3 Å. In addition, the α-C atoms of the phosphonates are even closer to that of 1, being ˜0.8 Å. The pyridine or quinoline group of these compounds is located in a big pocket defined by residues Met213, Trp211, Pro273, His208, His256, Asn210, Ser253, Ser150 and Glu151. Although the cavity has a mixed hydrophobic and polar feature, the ligands interact mainly with the hydrophobic side (i.e., Trp211 and Pro273). Each of the electron-deficient heterocyclic rings of 17-19 has a π-π stacking interaction with the electron-rich indole ring of Trp211, with the distance between the two parallel rings being ˜3.5 Å (FIG. 13b-d). Moreover, possible charge-transfer between the two electrostatically opposite rings makes the interaction particularly strong, which could elucidate why pyridine or quinoline phosphonates 15-19 have good inhibitory activity, while 13 is inactive. In all three DXR:phosphonate complexes, the hydroxamate binding site of 11s unoccupied. Moreover, there is no Mg²⁺ observed in these structures, despite 5 mM of MgCl₂ was present in the crystallization buffer. This could be due to the acidic crystallization condition (pH=5.6) used for the studies (Mac Sweeney et al., 2005) It is remarkable that in the absence of Mg²⁺, three carboxylates of metal binding residues Asp149, Glu151 and Glu230 are still positioned very close to those with a bound Mg²⁺ in the DXR:1 complex (FIG. 14).

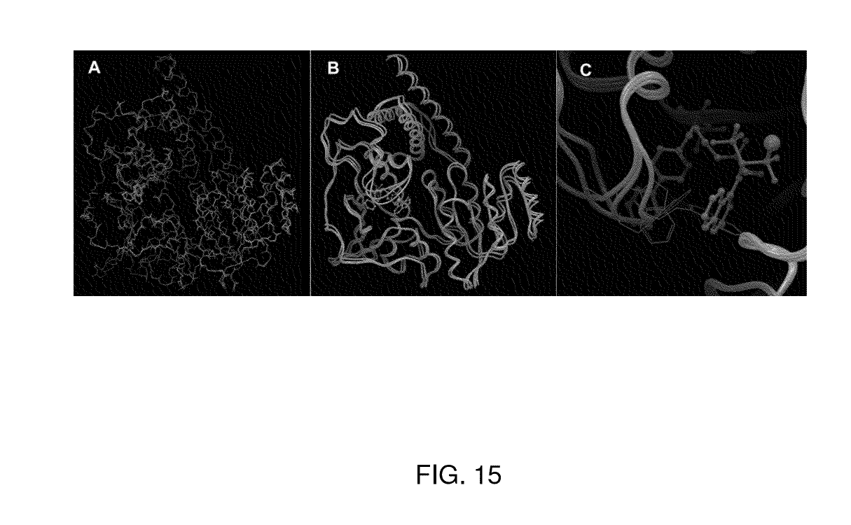

The overall 3-D structures of the DXR:17-19 complexes are almost identical (FIG. 15a), which is likely due to the structural similarity of the bound ligands. However, careful analysis of these three structures revealed that the conformation of the Trp211 sidechain in the DXR:18 complex is different from those of the DXR:17 and :19 complexes, with the indole ring flipped almost 180° (FIGS. 13b-d). This orientation could allow the 5-phenylpyridine group of 18 to have more π-π stacking as well as hydrophobic interactions with the indole ring, which might account for the enhanced inhibitory activity of 18. This finding as well as the π-π stacking/charge-transfer interactions discussed above indicate an important role of Trp211 in recognizing these lipophilic phosphonate inhibitors.

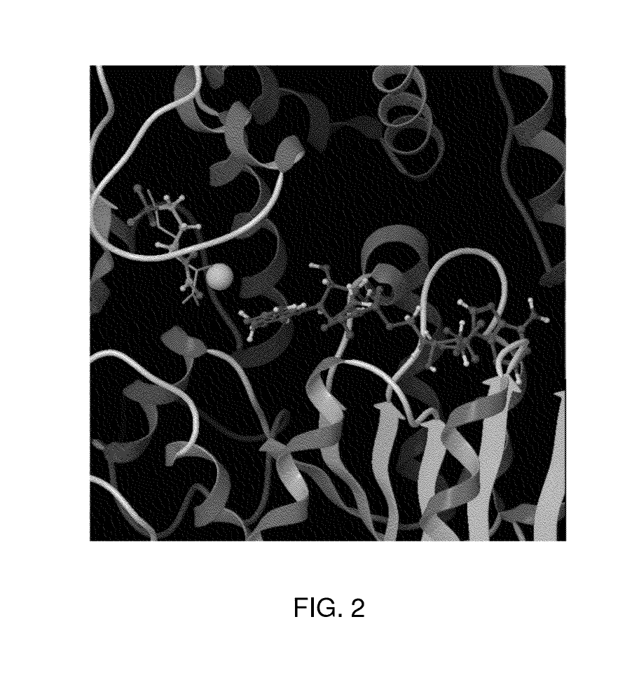

The inventors next examined functions of Trp211 in other available DXR:inhibitor complexes. As shown in FIG. 15b, superposition of the DXR:1 (yajima et al., 2007), DXR:921 and DXR:18 structures shows major conformational changes for a flexible loop containing residues 205-215 in the active site, while only subtle deviations are observed for the rest part of the protein backbone. Even more noticeable is the conformational change of the Trp211 indole ring, as shown in FIG. 2c. In the DXR:1 structure (in gray), since 1 has no hydrophobic group, the indole ring moves towards the center of the active site and covers on top of the carbon skeleton of 1, which is relatively hydrophobic. This allows the hydrophobic indole group to separate 1 from the solvent and largely close the active site, which might account for its potent inhibition. The crystal structures of DXR complexed with 18 and 9 demonstrate the flexible loop has great plasticity such that the Trp211 indole ring is able to move considerably in order to have favorable interactions with the lipophilic groups of these inhibitors (FIG. 15c). For instance, the indole N atoms of the DXR:18 and DXR:9 complexes are 4.6 Å and 4.4 Å away from that of the DXR:1 complex, respectively. In addition, each of the three planar indole rings adopts a different orientation, with one being almost vertical to the other two. It is also remarkable that in the DXR:bisphosphonate complexes, the Trp211 indole ring might also possess π-π stacking/charge transfer interactions with the electron-deficient isoquinoline (for 9) or pyridine ring (for 10), although there is a small angle of ˜15° between the two corresponding rings.

It is clear that Trp211 plays an important role in recognizing all of the DXR inhibitors with known crystal structures. This also provides an implication for future rational design of DXR inhibitors that an electron-deficient, hydrophobic group that has strong interactions with Trp211 could be favored. Moreover, Trp211 is conserved across the species using the MEP pathway. FIG. 6 shows a ClustalX27 alignment of DXR proteins from four representative species, i.e., E. coli, P. aeruginosa, Mycobacterium tuberculosis and P. falciparum, which are important human pathogens. Therefore, it is likely that inhibitors having strong interactions with Trp211 could exhibit a broad spectrum of activity.

The crystal structures of DXR:17-19 complexes reveal two mainly hydrophobic pockets, which are separated by the Trp211 indole ring. The conformational change of Trp211 opens up a large, mainly hydrophobic pocket (Pocket A), surrounded by Trp211, Pro273, His208, His256, Asn210, Ser253 and Ser150. In the previously reported structures, the corresponding pocket A is largely compressed due to the orientation of the Trp211 sidechain and thus poorly defined. This cavity could be of importance with respect to lipophilic inhibitor design, since it is right adjacent to the substrate binding site of DXR. Pocket B is surrounded by the residues Trp211, Met 213, Met275 and Ser150, which is not occupied by 17-19. Rather, this pocket is the binding site of the hydrophobic groups of 9 and 10.

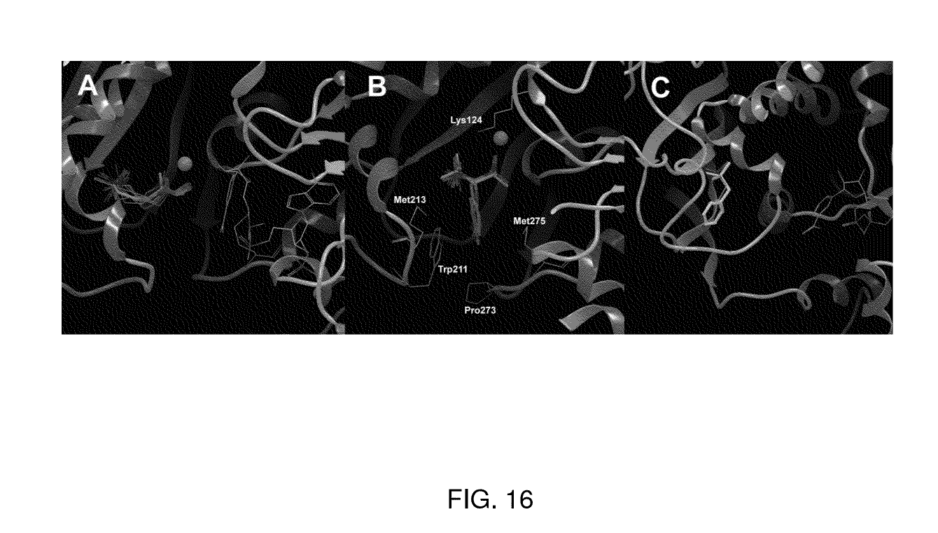

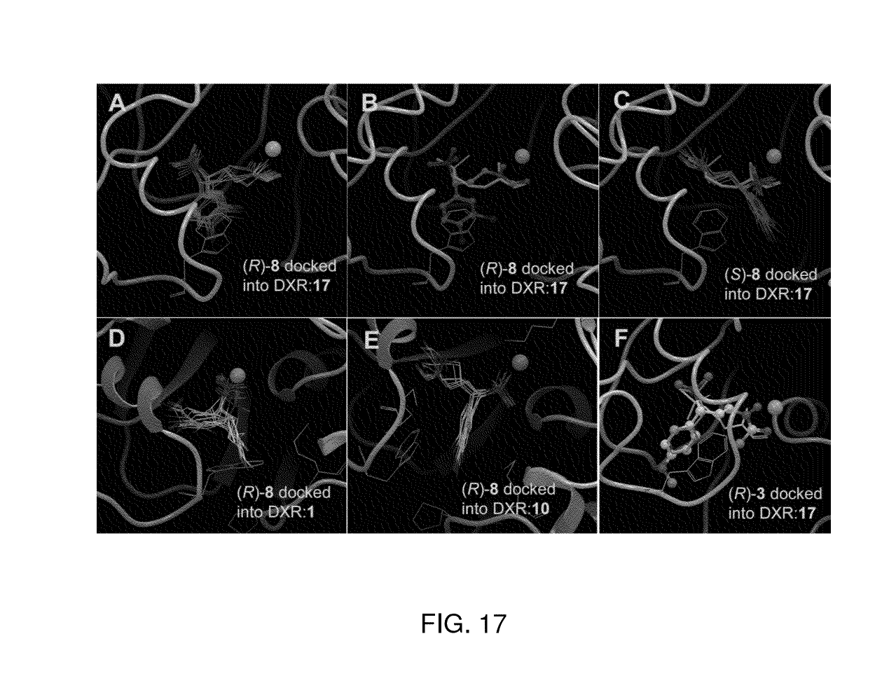

Due to the structural similarity of 17-19 to compound 8, our structures could be useful templates for modeling the binding structures of 8 and other lipophilic inhibitors to DXR. Glide in Schrödinger Suite (version: 2009) (Glide, version 5.5, Schrödinger, LLC) was used to perform the docking studies. First, Glide well predicted the binding modes of 1, 10 and 17, as shown in FIG. 16. For each of these compounds, the 10 docking structures with lowest energies are tightly clustered to each other with rms (root mean square) deviations ranging from 0.37 to 1.9 Å. They also show the same key interactions with the enzyme as the crystal structures. For example, all of the hydroxamate groups of 1 chelate the Mg²⁺, which is the “gold” standard for docking a hydroxamate ligand into a metalloenzyme. Upon validating Glide, we next used the DXR:17 structure to model the binding mode of compound 8. Since a Mg²⁺ is needed for successful docking of 8 having a hydroxamate group, we extracted the Mg²⁺ from the DXR:1 structure, added it into the DXR:17 structure, and the new structure was energy-minimized. This is reasonable since the conformations of Mg²⁺-binding residues Asp149, Glu151 and Glu230 do not change significantly (FIG. 14). As 8 contains an asymmetric α-carbon atom, the structures of both enantiomers, (R)- and (S)-8, were docked into the DXR:17 structure with the Mg²⁺ and the results are shown in FIG. 17a-c. (R)-8 can be docked very favorably, with 10 docking structures tightly clustered with each other and mimic the binding mode of 1 (FIG. 17a,b). All of the phosphonate groups are located in the phosphonate binding site of 1 and each of the hydroxamate moieties chelates the Mg²⁺. In addition, all of the 3,4-dichlorophenyl groups occupy pocket A and have π-π stacking interactions with the indole ring of Trp211. However, docking (S)-8 yielded a relatively poor result (FIG. 17c). All of the 10 docking structures with lowest energies show a “reversed” binding mode with the phosphonate binding to Mg²⁺ and the hydroxamate located in the phosphonate binding site of 1. In addition, their 3,4-dichlorophenyl groups are in pocket B without interactions with Trp211.

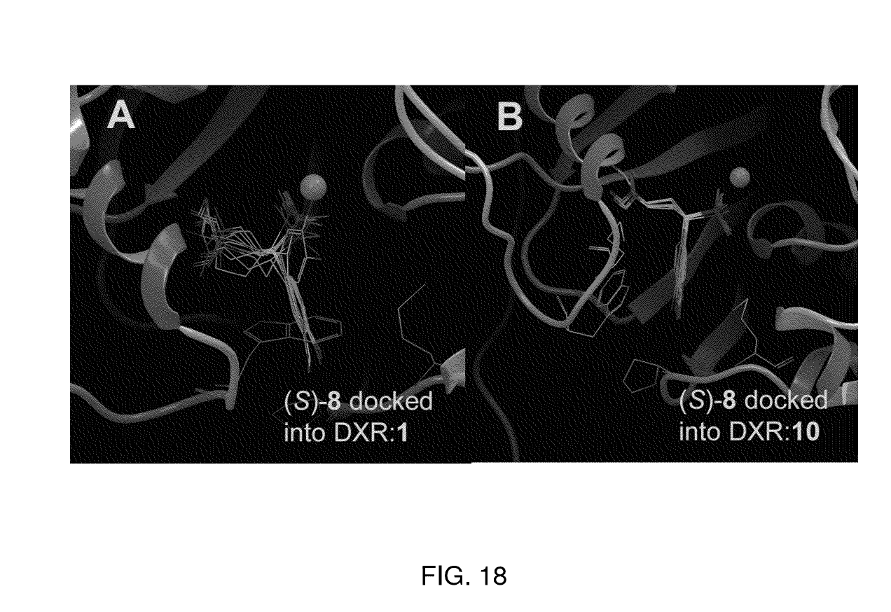

Docking studies of 8 using the DXR:1 and DXR:10 structures were also performed as a comparison. In both cases, (R)- and (S)-8 are predicted to adopt a “reversed” binding style (FIG. 17d,e and FIG. 18), with all of the phosphonate groups binding to Mg²⁺. The hydroxamate groups are mostly located in the phosphonate binding site of 1 and the lipophilic 3,4-dichlorophenyl group found in Pocket B. The docking results of 8 indicate our phosphonate:DXR structures are a good platform for modeling the binding structure of 8. Interestingly, these results also indicate that (R)-8 might have better inhibitory activity than its (S)-enantiomer, which is also implied by superimposing the DXR:17 to the DXR:1 structure (FIG. 13a). Should the pyridin-2-yl group have been attached to the α-position of 1, the virtually generated molecule would have a R-configuration.

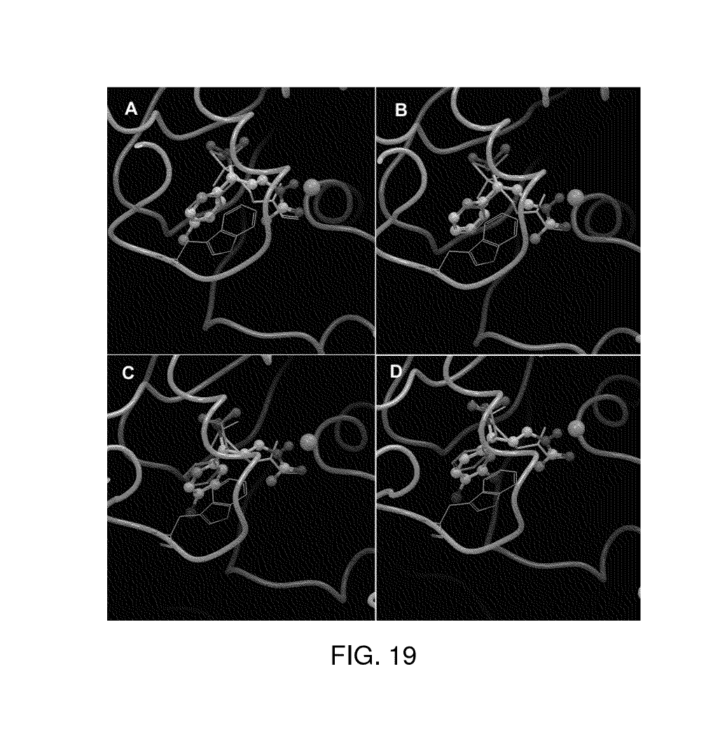

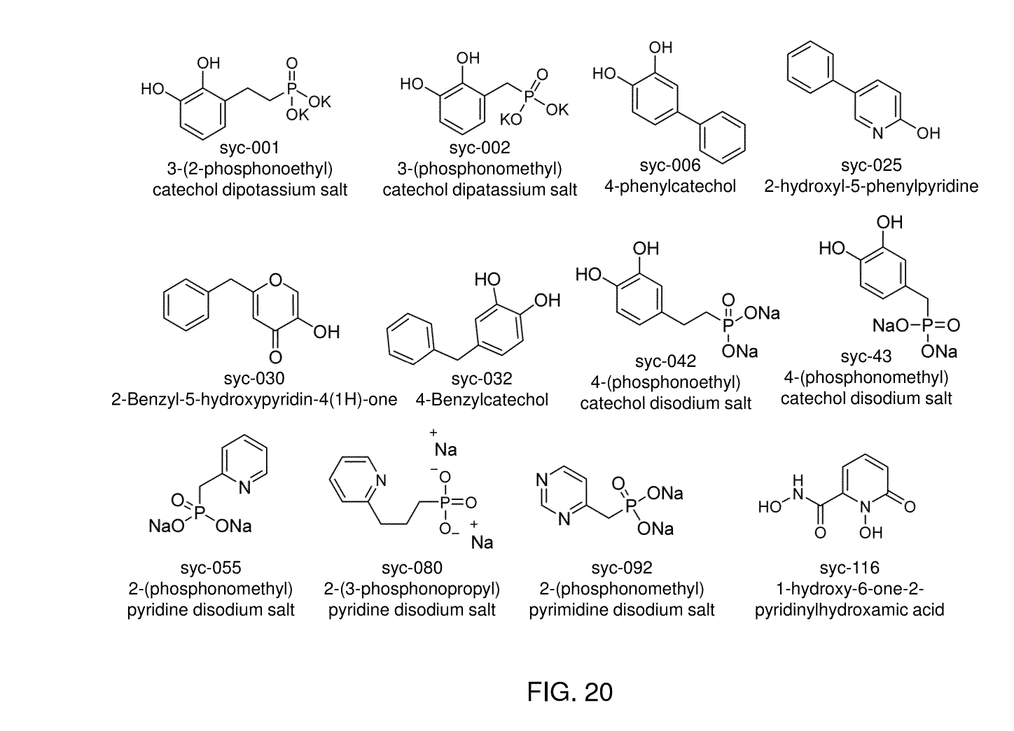

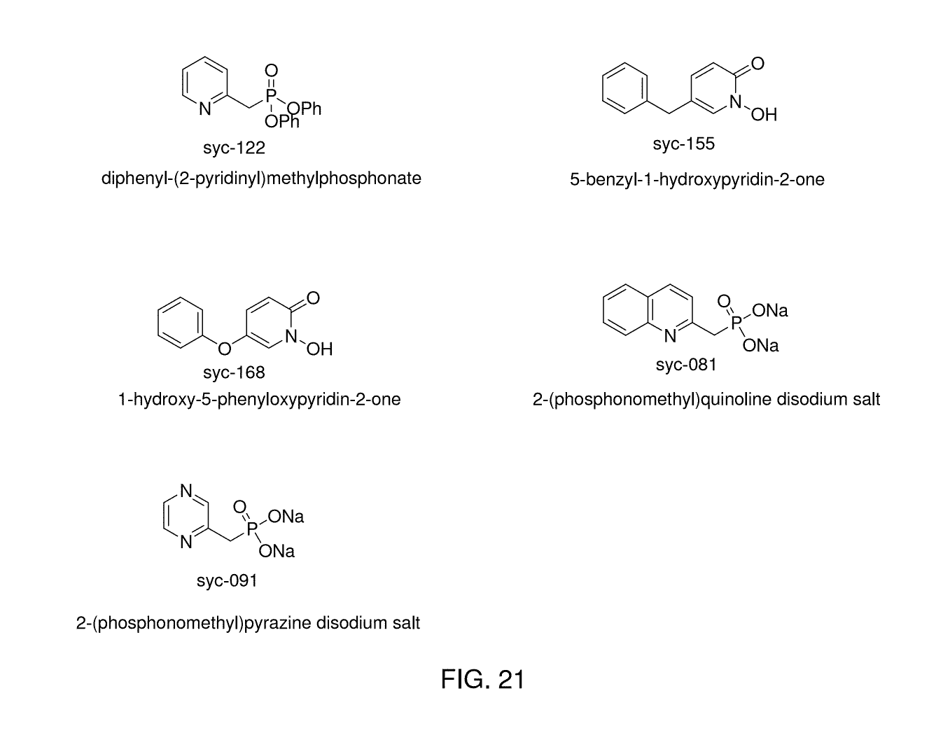

Moreover, docking studies of compounds 3-7 yielded similar outcomes. The (R)-enantiomers of these compounds can be docked favorably into the DXR structures, as shown in FIGS. 17f and 19. The moieties of 2 of these compounds are predicted to adopt the binding mode of 1, with the hydroxamate groups chelating the Mg²⁺. Their hydrophobic α-substituents are positioned in pocket A and have favorable interactions with Trp211. It is noteworthy that the docking results are in agreement with previously reported SAR of 3-7.11 The generally increasing activities from 3 to 7 (4-OMe-Ph<4-Me-Ph<H-Ph<4-Cl-Ph≈3,4-diCl-Ph) are correlated to the interactions of these aromatic sidechain with the electron-rich Trp211 indole ring. The crystallographic and modeling studies now provide a structural basis to elucidate these experimental data.

Finally, the inventors describe the synthesis of 12-19 with the general methods shown in Scheme 1. Pyridine-4-carbaldehyde was treated with the sodium salt of triethyl phosphonoacetate to give an α,β-unsaturated ester. It was hydrogenated, reduced to its corresponding alcohol with LiAlH4, and then oxidized to an aldehyde, which subjected to reductive amination with hydroxylamine and NaBH₃CN to afford an N-substituted hydroxylamine. It was formylated using formyl acetyl mixed anhydride to give the product 14. Compound 12 can be made in the same manner from 3,4-dichlorobenzaldehyde. A pyridine aldehyde was reduced and converted to the corresponding pyridinylmethyl chloride, which was then heated with triethyl phosphite at 120° C. overnight to give phosphonate diethyl ester. It was then treated with bromotrimethylsilane to afford compounds 15-19. Compound 13 was similarly prepared from 3,4-dichlorobenzaldehyde.

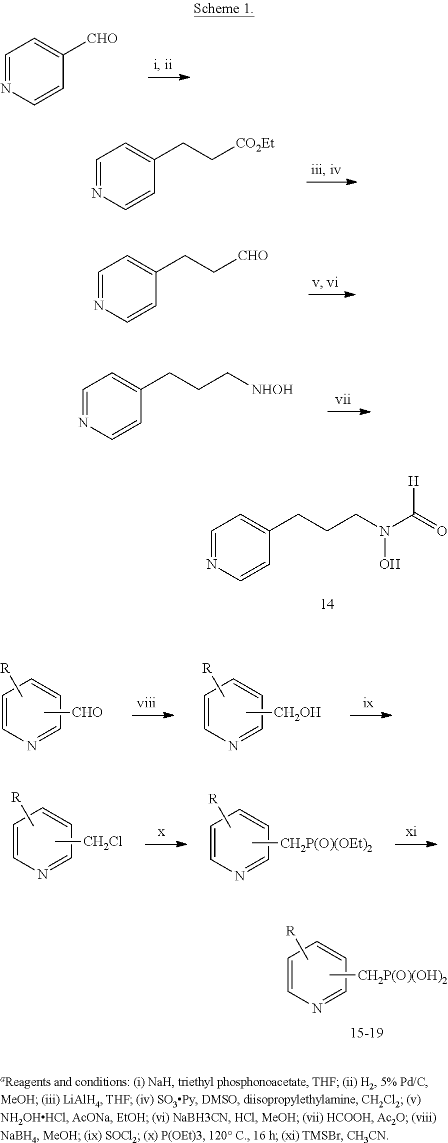

In summary, potent lipophilic DXR inhibitors are needed for their potential to become new drugs to treat drug-resistant bacterial and malarial infections. However, structural insight into the hydrophobic nature of DXR active site remains unclear. We have found pyridine/quinoline containing phosphonates 15-19 to be a new class of DXR inhibitors (IC₅₀s being as low as 0.84 μM), which can be used as lipophilic probes for such structural studies. Second, we solved the crystal structures of three DXR:phosphonate complexes that reveal a novel inhibitor binding mode. Their phosphonate groups occupy the phosphonate binding site of 1. Of particular interest is that the conformation of the DXR flexible loop is changed considerably, with the indole ring of Trp211 moving ˜4.6 Å to open up a large, mainly hydrophobic pocket. The electron-deficient pyridine/quinoline rings of these inhibitors are located in this newly formed cavity and have strong π-π stacking/charge-transfer interactions with the electron-rich indole ring. Analysis of all DXR:inhibitor structures uncovers the critical role of Trp211 in inhibitor recognition and binding. Third, docking studies show our crystal structures could be useful for predicting the binding modes of compound 8 as well as its analogs 3-7. The modeling shows that their backbones adopt the binding style of 1 and their hydrophobic sidechains have favorable interactions with Trp211. Moreover, this model is further supported by the structure activity relationship of these inhibitors. Overall, this work reveals an important role of Trp211 in inhibitor recognition and provides a structural basis for future design and development of lipophilic DXR inhibitors.

### Experimental Section

All reagents were purchased from Alfa Aesar (Ward Hill, Mass.) or Aldrich (Milwaukee, Wis.). All compounds were characterized by ¹H, ¹³C and ³¹P NMR on a Varian (Palo Alto, Calif.) 400-MR spectrometer and the purities monitored by using ¹H (at 400 MHz) absolute spin-count quantitative NMR analysis with imidazole as an internal standard. The purities of compounds 12-19 were found to be >95%. High resolution mass spectra of compounds with important biological activity were measure on a ThermoFisher LTQ-Orbitrap mass spectrometer.

General method for synthesis of compounds 12 and 14. To a solution of triethyl phosphonoacetate (4.4 mL, 22 mmol) in THF (50 mL) was added NaH (968 mg, 24.4 mmol, 60% in oil) at 0° C. Aldehyde (20 mmol) was added to the resulting solution. After being stirred for 2 h at room temperature, the reaction mixture was quenched with saturated NH₄Cl and extracted with Et2O (3×25 mL). The combined organic phases were dried over Na₂SO₄, filtered and concentrated under reduced pressure. The crude product was hydrogenated with 5% Pd/C in methanol (40 mL) for 2 h to afford the desired saturated ester, which was then reduced with LiAlH₄ (30 mmol) in THF (60 mL) at room temperature to yield the primary alcohol. The resulting alcohol (3 mmol) was dissolved in a mixture of CH₂Cl₂ (10 mL), DMSO (2.14 mL, 15 mmol) and diisopropylethylamine (2.57 mL, 30 mmol). SO₃.Py (1.46 g, 9 mmol) was added in one portion at 0° C. and the reaction mixture was stirred for 5 h at room temperature. The reaction was quenched with cold water (10 mL) and extracted with CH₂Cl₂ (3×15 mL). The combined organic layers were dried over NaSO₄, filtered and concentrated under vacuum. The residue was purified by flash column chromatography (silica gel) to give an aldehyde. To a solution of the aldehyde thus obtained (2 mmol) in EtOH (6 mL) were added AcONa (492 mg, 6 mmol) and NH₂OH—HCl (153 mg, 2.2 mmol) successively. The reaction mixture was stirred for 3 h at room temperature and then concentrated. The crude oil was treated with water and extracted with EtOAc (3×8 mL). The combined organic layers were dried over NaSO₄, filtered and concentrated under reduced pressure to give the corresponding oxime, which can be used for the next step without further purification. It was dissolved in anhydrous MeOH (5 mL) and NaBH₃CN (138.2 mg, 2.2 mmol) then added, followed by addition of 4 M HCl in dioxane until pH=3. The reaction mixture was stirred overnight and the solvent removed under vacuum. EtOAc (35 mL) was added to the residue and the solution neutralized with 2 M NaOH to pH ˜8. Upon removal of the solvent under vacuum, the residue was purified by flash column chromatography (silica gel) to give a hydroxylamine. It (1 mmol) was added to a formic acid (1 mL) solution containing acetyl anhydride (2 mmol) at room temperature and stirred overnight. The reaction mixture was concentrated and purified with flash column chromatography (silica gel) to give the final products.

N-[3-(3,4-Dichlorophenyl)propyl]-N-hydroxyformamide (12): It was prepared following above general method as a colorless oil in an overall 41% yield from 3,4-dichlorobenzaldehyde. NMR showed it exists as a mixture of two rotamers with ˜6:4 ratio. ¹H NMR (400 MHz, CDCl₃): δ 8.16 (brs. 0.6H), 8.03 (brs. 0.4H), 7.35-7.32 (m, 2H), 7.0 (d, J=6.4 Hz, 1H), 3.74-3.69 (m, 2H), 2.64-2.59 (m, 2H), 1.95-1.85 (m, 2H); ¹³C NMR (100 MHz, CDCl₃): δ 162.2 (minor), 159.0 (major), 141.2, 132.3, 130.5, 130.4, 130.3, 127.9, 48.6 (major), 46.5 (minor), 31.8 (major), 31.4 (minor), 21.1 (minor), 20.7 (major); HRMS (ESI) calcd for C₁₀H₁₂NCl₂O₂ [M+H]⁺: 248.0245. found: 248.0238.

N-Hydroxyl-N-[3-(pyridin-4-yl)propyl]formamide (14): It was prepared following above general method as a colorless oil in an overall 18% yield from pyridine-4-carbaldehyde. NMR showed it exists as a mixture of two rotamers with ˜5:2 ratio. ¹H NMR (400 MHz, CDCl₃): δ 8.46 (d, J=6.4 Hz, 1.44H), 8.37 (d, J=6.4 Hz, 0.56H), 7.86 (s, 1H), 7.15 (d, J=6.4 Hz, 1.44H), 7.11 (d, J=6.4 Hz, 0.56H), 3.65 (t, J=6.8 Hz, 0.56H), 3.53 (t, J=6.8 Hz, 1.44H), 2.69-2.63 (m, 2H), 2.10-2.05 (m, 1.55H), 2.0-1.93 (m, 0.55H); ¹³C NMR (100 MHz, CDCl₃): δ 162.8 (minor), 156.3 (major), 149.2 (major), 148.5 (minor), 124.3 (minor), 124.1 (major), 48.8 (major), 45.9 (minor), 32.0 (minor), 31.7 (major), 27.3 (major), 26.9 (minor); HRMS (ESI) calcd for C₉H₁₃N₂O₂ [M+H]⁺: 181.0977. found: 181.0969.

General method for synthesis of phosphonic acids 13, 15-19. To a solution of an aldehyde (6 mmol) in methanol (12 mL) was slowly added NaBH₄ (342 mg, 9 mmol) at 0° C. After 1 h at room temperature, the solvent was removed under reduced pressure and the reaction was quenched with water and extracted with EtOAc (3×20 mL). The combined organic phases were dried over NaSO₄, filtered and concentrated under reduced pressure. The resulting crude alcohol was dissolved in CH₂Cl₂ (15 mL) and a solution of SOCl2 (1.3 mL, 18 mmol) in CH₂Cl₂ (5 mL) was then added slowly at 0° C. After stirring 3 h at room temperature, the solvent was removed under vacuum. The residue was treated with saturated Na₂CO₃ (10 mL) and extracted with CH₂Cl₂ (3×10 mL). The combined organic layers were dried, filtered and evaporated. The crude chloride was heated with triethyl phosphite (18 mmol) at 120° C. under N₂ overnight. Upon removal of excess triethyl phosphite under reduced pressure, the residue was purified with flash column chromatography (silica gel) to give the corresponding phosphonic acid diethyl ester. The phosphonate thus obtained (1.83 mmol) was treated with TMSBr (1.2 mL, 9.2 mmol) in anhydrous CH₃CN (8 mL) at room temperature for 8 h. The solution was evaporated to dryness and MeOH (5 mL) added to the residue. The solvent was then removed under reduced pressure again and the residue redissolved in MeOH (5 mL). Neutralization with 2 M NaOH to pH ˜9 gave the disodium salt of a phosphonic acid as a white powder, which may be recrystallized in H₂O/acetone if necessary.

(3,4-Dichlorophenyl)methylphosphonic acid (13): It was prepared following above general method as a white powder in an overall 79% yield from 3,4-dichlorobenzaldehyde. ¹H NMR (400 MHz, D₂O): δ 7.33-7.31 (m, 2H), 7.05 (d, J=8.4 Hz, 1H), 2.91 (d, J=20.8 Hz, 2H); ¹³C NMR (100 MHz, D₂O): δ 141.7 (d, J=7.7 Hz), 133.5, 133.2, 132.1, 131.8, 130.4, 39.0 (d, J=121.3 Hz); ³¹P NMR (162 MHz, D₂O): δ 21.1.

Pyridin-4-ylmethylphosphonic acid disodium salt (15): It was prepared following above general method as a white powder in an overall 56% yield from pyridine-4-carbaldehyde. ¹H NMR (400 MHz, D₂O): δ 8.21 (d, J=4.8 Hz, 2H), 7.23 (d, J=4.8 Hz, 2H), 2.76 (d, J=20 Hz, 2H); ¹³C NMR (100 MHz, D₂O): δ 147.8, 125.3, 125.4, 37.3 (d, J=116.1 Hz); ³¹P NMR (162 MHz, D₂O): δ 17.9; HRMS (ESI) calcd for C₆H₉NO₃P [M+H]⁺: 174.0320. found: 174.0308.

Pyridin-3-ylmethylphosphonic acid disodium salt (16): It was prepared following above general method as a white powder in an overall 62% yield from pyridine-3-carbaldehyde. ¹H NMR (400 MHz, D₂O): δ 8.23 (d, J=4.0 Hz, 1H), 7.62 (t, J=8.0 Hz, 1H), 7.36 (d, J=8.0 Hz, 1H), 7.10 (t, J=6.0 Hz, 1H), 2.93 (d, J=19.6 Hz, 2H); ¹³C NMR (100 MHz, D₂O): δ 160.3, 150.1, 139.8, 126.8, 123.5, 42.1 (d, J=117.6 Hz); ³¹P NMR (162 MHz, D₂O): δ 18.5; HRMS (ESI) calcd for C₆H₉NO₃P [M+H]⁺: 174.0323. found: 174.0310.

Pyridin-2-ylmethylphosphonic acid disodium salt (17): It was prepared following above general method as a white powder in an overall 53% yield from pyridine-2-carbaldehyde. ¹H NMR (400 MHz, D₂O): δ 8.30 (d, J=4.4 Hz, 1H), 7.86 (m, 1H), 7.46 (m, 1H), 7.33 (d, J=6.0 Hz, 1H), 3.06 (d, J=19.2 Hz, 2H); ¹³C NMR (100 MHz, D₂O): δ 157.8, 147.5, 143.5, 128.4, 124.9, 40.3 (d, J=114.6 Hz); ³¹P NMR (162 MHz, D₂O): δ 17.5; HRMS (ESI) calcd for C₆H₉NO₃P [M+H]⁺: 174.0318. found: 174.0310.

(5-Phenylpyridin-2-yl)methylphosphonic acid disodium salt (18): It was prepared following above general method as a white powder in an overall 67% yield from 5-phenylpyridine-2-carbaldehyde. ¹H NMR (400 MHz, D₂O): δ 8.51 (s, 1H), 7.84 (d, J=4.8 Hz, 1H), 7.59 (s, 1H), 7.57 (s, 1H), 7.45-7.31 (m, 4H), 2.99 (d, J=20 Hz, 2H); ¹³C NMR (100 MHz, D₂O): δ 156.9, 145.7, 137.3, 135.4, 133.3, 133.2, 129.1, 127.9, 126.7, 124.2, 124.1, 40.1 (d, J=115.6 Hz); ³¹P NMR (162 MHz, D₂O): δ 15.6; HRMS (ESI) calcd for Cl₂H₁₃O₃NP [M+Na]⁺: 272.0453. found: 272.0440.

Quinolin-2-ylmethylphosphonic acid disodium salt (19): It was prepared following above general method as an off-white powder in an overall 58% yield from quinoline-2-carbaldehyde. ¹H NMR (400 MHz, D₂O): δ 8.16 (d, J=8.4 Hz, 1H), 7.84-7.80 (m, 2H), 7.62 (t, J=7.2 Hz, 1H), 7.52 (d, J=8.4 Hz, 1H), 7.46 (t, J=7.2 Hz, 1H), 3.17 (d, J=20 Hz, 2H); ¹³C NMR (100 MHz, D₂O): δ 162.1, 148.7, 139.3, 132.2, 130.4, 128.9, 128.8, 128.3, 125.3, 43.4 (d, J=115.3 Hz); ³¹P NMR (162 MHz, D₂O): δ 14.8; HRMS (ESI) calcd for C₁₀H₁₁NO₃P [M+Na]: 246.0296. found: 246.0286.

Crystallization. The crystallization of E. coli DXR was performed as described previously (Yajima et al., 2004). Purified His×6-tagged protein was crystallized under the condition of 0.1 M sodium citrate buffer pH 5.6, 0.8 M sodium malonate, and 0.3 M potassium sodium (+)-tartrate with 3 mM NADPH and 5 mM MgCl₂. The crystals were soaked for 1 min with inhibitors (0.2 M) dissolved in the crystallization buffer containing 28% glycerol as a cryoprotectant. Data were collected at 2.1 Å (for 17) and 2.0 Å-resolution (for 18, 19) at NW12A, Photon Factory, which were then processed using the program HKL2000 (Borek et al., 2003 and the initial structures obtained by the program Molrep (Vagin and Teplyakov, 1997) using the coordinates of 2EGH as a target. The refinement was carried out with the program CNS (Brunger et al., 1998; Brunger, 2007) once at first, then by the program Refmac5 (Murshudov et al., 1997) iteratively. The final refinement statistics were summarized in Table 2 and the coordinates were deposited into Protein Data Bank as 3ANL, 3ANM, and 3ANN for DXR-17, -18 and -19 complexes, respectively.

Molecular modeling. Molecular modeling and docking studies were performed using Schrödinger suite 2009 (Schrödinger Suite 2009), which includes all of the programs described below. Crystal structures of E. coli DXR were prepared using the module “protein preparation wizard” in Maestro 9.0 with default protein parameters: water molecules (>3.0 Å away from a ligand) were removed, hydrogen atoms added, inhibitors (e.g., fosmidomycin) extracted, and the Mg²⁺ ion and NADPH (if present) remained in the protein structure for docking. H-bonds were then optimized and the protein was energy-minimized using OPLS-2005 force field. A receptor grid, which is large enough to contain the whole active site, was generated using Glide without any constraints. Compounds were built, minimized using OPLS-2005 force field in Maestro, and further minimized using Juguar (with a basis set of 6-31G**) to obtain more accurate partial charge of each atom. The molecules were then docked into the prepared protein structure using Glide (docking parameters: standard-precision and dock flexibly).

DXR inhibition assay. E. coli DXR expression and purification were carried out as previously reported (Deng et al., 2009). In brief, E. coli M15 strain (Qiagen) was transformed and cultured at 37° C. in LB medium containing kanamycin (25 μg/mL) and ampicillin (50 μg/mL). Upon reaching an optical density of ˜0.6 at 600 nm, DXR expression was induced for 5 hours by adding 0.2 mM isopropylthiogalactoside. Cells were then harvested and resuspended in 100 mM Tris-HCl (pH 8.0). After sonication, the lysate was centrifuged at 15,000 g for 20 min and the supernatant was collected and subjected to an affinity column chromatography using Ni-NTA resin (GE Healthcare). The resin was washed with 50 mM imidazole in 100 mM Tris-HCl (pH 8.0) and then the protein was eluted with 200 mM imidazole in 100 mM Tris-HCl (pH 8.0). After desalting (using HiTrap, GE Healthcare) to remove excess imidazole, the protein was concentrated and stored in small aliquots at −80° C.

The enzyme assay was also performed as previously described (Deng et al., 2009) in a 96-well microplate using 2 nM enzyme, 2 mM MgCl₂, 100 μM DXP, 50 μM NADPH in 50 mM HEPES buffer (pH=7.5) containing 25 μg/mL BSA. For inhibition assay, compounds were pre-incubated with DXR for 20 min at 30° C., before adding DXP to initiate the reaction. The process was monitored at 340 nm with a Beckman DTX-880 microplate reader. The initial velocities of wells containing increasing concentrations of an inhibitor were calculated and input into Prism (version 3.0, GraphPad Software, Inc., La Jolla, Calif.). The IC₅₀ values were then calculated by using a standard dose response curve fitting in the software. The reported IC₅₀s were the mean values of at least two independent experiments.

### REFERENCES

All patents and publications mentioned in the specifications are indicative of the levels of those skilled in the art to which the invention pertains. All patents and publications are herein incorporated by reference to the same extent as if each individual publication was specifically and individually indicated to be incorporated by reference.
A call to arms. Nat Rev Drug Discov 2007, 6, 8-12
Agrawal, Y. K.; Roshania, R. D. Z. Phys. Chemie, Leipzig 1982, 263, 822-826.
Altincicek, B.; Hintz, M.; Sanderbrand, S.; Wiesner, J.; Beck, E.; Jomaa, H. Tools for discovery of inhibitors of the 1-deoxy-D-xylulose 5-phosphate (DXP) synthase and DXP reductoisomerase: an approach with enzymes from the pathogenic bacterium Pseudomonas aeruginosa. FEMS Microbiol Lett 2000, 190, 329-33
Athavale, V. T.; Prabhu, L. H.; Vartak, D. G. J. Inorg. Nucl. Chem. 1966, 28, 1237-1249.
Bock, V.; Klotzer, W.; Singewald, N.; Strieder, G. Synthesis 1987, 1058-60.
Borek, D.; Minor, W.; Otwinowski, Z. Measurement errors and their consequences in protein crystallography. Acta Crystallogr. D Biol. Crystallogr. 2003, 59, 2031-2038
Borrmann, S.; Adegnika, A. A.; Matsiegui, P. B.; Issifou, S.; Schindler, A.; Mawili-Mboumba, D. P.; Baranek, T.; Wiesner, J.; Jomaa, H.; Kremsner, P. G. Fosmidomycin-clindamycin for Plasmodium falciparum Infections in African children. J Infect Dis 2004, 189, 901-8
Borrmann, S.; Adegnika, A. A.; Moussavou, F.; Oyakhirome, S.; Esser, G.; Matsiegui, P. B.; Ramharter, M.; Lundgren, I.; Kombila, M.; Issifou, S.; Hutchinson, D.; Wiesner, J.; Jomaa, H.; Kremsner, P. G. Short-course regimens of artesunate-fosmidomycin in treatment of uncomplicated Plasmodium falciparum malaria. Antimicrob Agents Chemother 2005, 49, 3749-54
Borrmann, S.; Lundgren, I.; Oyakhirome, S.; Impouma, B.; Matsiegui, P. B.; Adegnika, A. A.; Issifou, S.; Kun, J. F.; Hutchinson, D.; Wiesner, J.; Jomaa, H.; Kremsner, P. G. Fosmidomycin plus clindamycin for treatment of pediatric patients aged 1 to 14 years with Plasmodium falciparum malaria. Antimicrob Agents Chemother 2006, 50, 2713-8
Brown, A. C.; Parish, T. BMC Microbiol. 2008, 8, 78; (c) Sakamoto, Y.; Furukawa, S.; Ogihara, H.; Yamasaki, M. Biosci. Biotechnol. Biochem. 2003, 67, 2030-3
Brown, A. C.; Parish, T. DXR is essential in Mycobacterium tuberculosis and fosmidomycin resistance is due to a lack of uptake. BMC Microbiol. 2008, 8, 78-86.
Brunger, A. T.; Adams, P. D.; Clore, G. M.; DeLano, W. L.; Gros, P.; Grosse-Kunstleve, R. W.; Jiang, J. S.; Kuszewski, J.; Nilges, M.; Pannu, N. S.; Read, R. J.; Rice, L. M.; Simonson, T.; Warren, G. L. Crystallography & NMR system: A new software suite for macromolecular structure determination. Acta Crystallogr. D Biol. Crystallogr. 1998, 54, 905-921; (b) Brunger, A. T. Version 1.2 of the Crystallography and NMR system. Nat. Protoc. 2007, 2, 2728-2733
Carretero-Paulet, L.; Ahumada, I.; Cunillera, N.; Rodriguez-Concepcion, M.; Ferrer, A.; Boronat, A.; Campos, N. Expression and molecular analysis of the Arabidopsis DXR gene encoding 1-deoxy-D-xylulose 5-phosphate reductoisomerase, the first committed enzyme of the 2-C-methyl-D-erythritol 4-phosphate pathway. Plant Physiol 2002, 129, 1581-91
Chen, C. K. M.; Hudock, M. P.; Zhang, Y.; Guo, R. T.; Cao, R.; No, J. H.; Liang, P. H.; Ko, T. P.; Chang, T. H.; Chang, S. C.; Song, Y.; Axelson, J.; Kumar, A.; Wang, A. H.; Oldfield, E. Inhibition of geranylgeranyl diphosphate synthase by bisphosphonates: a crystallographic and computational investigation. J Med Chem 2008, 51, 5594-607
Christoffersen, R. E. Antibiotics—an investment worth making? Nat Biotechnol 2006, 24, 1512-4
Cramer, R. D., 3rd; Patterson, D. E.; Bunce, J. D. Recent advances in comparative molecular field analysis (CoMFA). Prog Clin Biol Res 1989, 291, 161-5
Deng, L.; Sundriyal, S.; Rubio, V.; Shi, Z.; Song, Y. Coordination Chemistry Based Approach to Lipophilic Inhibitors of 1-Deoxy-D-xylulose-5-phosphate Reductoisomerase. J. Med. Chem., 2009, 52, 6539-6542
Devreux, V.; Wiesner, J.; Jomaa, H.; Rozenski, J.; Van der Eycken, J.; Van Calenbergh, S. Divergent strategy for the synthesis of α-aryl-substituted fosmidomycin analogues. J. Org. Chem. 2007, 72, 3783-3789
Dhiman, R. K.; Schaeffer, M. L.; Bailey, A. M.; Testa, C. A.; Scherman, H.; Crick, D. C. 1-Deoxy-D-xylulose 5-phosphate reductoisomerase (IspC) from Mycobacterium tuberculosis: towards understanding mycobacterial resistance to fosmidomycin. J. Bacteriol. 2005, 187, 8395-8402.
Driscoll, J. A.; Brody, S. L.; Kollef, M. H. The epidemiology, pathogenesis and treatment of Pseudomonas aeruginosa infections. Drugs 2007, 67, 351-68
Englebienne, P.; Fiaux, H.; Kuntz, D. A.; Corbeil, C. R.; Gerber-Lemaire, S.; Rose, D. R.; Moitessier, N. Evaluation of docking programs for predicting binding of Golgi alpha-mannosidase II inhibitors: a comparison with crystallography. Proteins 2007, 69, 160-76
Friesner, R. A.; Banks, J. L.; Murphy, R. B.; Halgren, T. A.; Klicic, J. J.; Mainz, D. T.; Repasky, M. P.; Knoll, E. H.; Shelley, M.; Perry, J. K.; Shaw, D. E.; Francis, P.; Shenkin, P. S. Glide: a new approach for rapid, accurate docking and scoring. 1. Method and assessment of docking accuracy. J Med Chem 2004, 47, 1739-49
Friesner, R. A.; Murphy, R. B.; Repasky, M. P.; Frye, L. L.; Greenwood, J. R.; Halgren, T. A.; Sanschagrin, P. C.; Mainz, D. T. Extra precision glide: docking and scoring incorporating a model of hydrophobic enclosure for protein-ligand complexes. J Med Chem 2006, 49, 6177-96
Giessmann, D.; Heidler, P.; Haemers, T.; Van Calenbergh, S.; Reichenberg, A.; Jomaa, H.; Weidemeyer, C.; Sanderbrand, S.; Wiesner, J.; Link, A. Towards new antimalarial drugs: synthesis of non-hydrolyzable phosphate mimics as feed for a predictive QSAR study on 1-deoxy-D-xylulose-5-phosphate reductoisomerase inhibitors. Chem Biodivers 2008, 5, 643-56
Glide, version 5.5, Schrödinger, LLC, New York, N.Y., 2009; Schrödinger Suite 2009, Schrödinger, LLC, New York, N.Y., 2009
Gottlin, E. B.; Benson, R. E.; Conary, S.; Antonio, B.; Duke, K.; Payne, E. S.; Ashraf, S. S.; Christensen, D. J. High-throughput screen for inhibitors of 1-deoxy-d-xylulose 5-phosphate reductoisomerase by surrogate ligand competition. J Biomol Screen 2003, 8, 332-9
Grolle, S.; Bringer-Meyer, S.; Sahm, H. Isolation of the dxr gene of Zymomonas mobilis and characterization of the 1-deoxy-D-xylulose 5-phosphate reductoisomerase. FEMS Microbiol Lett 2000, 191, 131-7
Guo, R. T.; Cao, R.; Liang, P. H.; Ko, T. P.; Chang, T. H.; Hudock, M. P.;
Jeng, W. Y.; Chen, C. K.; Zhang, Y.; Song, Y.; Kuo, C. J.; Yin, F.; Oldfield, E.; Wang, A. H. Bisphosphonates target multiple sites in both cis- and trans-prenyltransferases. Proc Natl Acad Sci USA 2007, 104, 10022-7
Haemers, T.; Wiesner, J.; Van Poecke, S.; Goeman, J.; Henschker, D.; Beck, E.; Jomaa, H.; Van Calenbergh S. Synthesis of α-substituted fosmidomycin analogues as highly potent Plasmodium falciparum growth inhibitors. Bioorg. Med. Chem. Lett. 2006, 16, 1888-1891.
Henriksson, L. M.; Unge, T.; Carlsson, J.; Aqvist, J.; Mowbray, S. L.; Jones, T. A. Structures of Mycobacterium tuberculosis 1-deoxy-D-xylulose-5-phosphate reductoisomerase provide new insights into catalysis. J Biol Chem 2007, 282, 19905-16
http://162.129.70.33/images/Pseudomonas_Aeruginosa_in_burn_{—}1_{—}060529.jpg.
http://de.wikipedia.org/wiki/Bild:Pseudomonas.jpg.
http://www.hoinews.com/uploadedImages/weyi/News/Stories/Tuberculosis-x-ray-1.jpg.
http://www.schrodinger.com/
http://www.sumivector.com/images/stories/medium/plasmodium-falciparum_med.jpg
http://www.who.int/research/en/.
http://www.who.int/tb/publications/2008/drs_report4_{—}26feb08.pdf
Hudock, M. P.; Sanz-Rodriguez, C. E.; Song, Y.; Chan, J. M.; Zhang, Y.; Odeh, S.; Kosztowski, T.; Leon-Rossell, A.; Concepcion, J. L.; Yardley, V.; Croft, S. L.; Urbina, J. A.; Oldfield, E. Inhibition of Trypanosoma cruzi hexokinase by bisphosphonates. J Med Chem 2006, 49, 215-23
Hunter, W. N.; The non-mevalonate pathway of isoprenoid precursor biosynthesis. J. Biol. Chem. 2007, 282, 21573-21577.
Jacobsen, F. E.; Lewis, J. A.; Cohen, S. M. J. Am. Chem. Soc. 2006, 128, 3156-7
Jacobsen, F. E.; Lewis, J. A.; Cohen, S. M. ChemMedChem 2007, 2, 152-171
Jomaa, H.; Wiesner, J.; Sanderbrand, S.; Altincicek, B.; Weidemeyer, C.; Hintz, M.; Turbachova, I.; Eberl, M.; Zeidler, J.; Lichtenthaler, H. K.; Soldati, D.; Beck, E., Inhibitors of the nonmevalonate pathway of isoprenoid biosynthesis as antimalarial drugs. Science 1999, 285, 1573-1576.
Jones, J. L.; Kruszon-Moran, D.; Sanders-Lewis, K.; Wilson, M. Toxoplasma gondii infection in the United States, 1999 2004, decline from the prior decade. Am J Trop Med Hyg 2007, 77, 405-10
Klebe, G.; Abraham, U.; Mietzner, T. Molecular similarity indices in a comparative analysis (CoMSIA) of drug molecules to correlate and predict their biological activity. J Med Chem 1994, 37, 4130-46
Kontoyianni, M.; McClellan, L. M.; Sokol, G. S. Evaluation of docking performance: comparative data on docking algorithms. J Med Chem 2004, 47, 558-65
Koppisch, A. T.; Fox, D. T.; Blagg, B. S.; Poulter, C. D. E. coli MEP synthase: steady-state kinetic analysis and substrate binding. Biochemistry 2002, 41, 236-43
Kotsikorou, E.; Song, Y.; Chan, J. M.; Faelens, S.; Tovian, Z.; Broderick, E.; Bakalara, N.; Docampo, R.; Oldfield, E. Bisphosphonate inhibition of the exopolyphosphatase activity of the Trypanosoma brucei soluble vacuolar pyrophosphatase. J Med Chem 2005, 48, 6128-39
Kuntz, L.; Tritsch, D.; Grosdemange-Billiard, C.; Hemmerlin, A.; Willem, A.; Bach, T. J.; Rohmer, M. Isoprenoid biosynthesis as a target for antibacterial and antiparasitic drugs: phosphonohydroxamic acids as inhibitors of deoxyxylulose phosphate reducto-isomerase. Biochem. J. 2005, 386, 127-135.
Kurz, T.; Schluter, K.; Kaula, U.; Bergmann, B.; Walter, R. D.; Geffken D. Synthesis and antimalarial activity of chain substituted pivaloyloxymethyl ester analogues of Fosmidomycin and FR900098. Bioorg. Med. Chem. 2006, 14, 5121-5135.
Kuzuyama, T.; Shimizu, T.; Takahashi, S.; Seto, H. Fosmidomycin, a specific inhibitor of 1-deoxy-xylulose 5-phosphate reductoisomerase in the nonmevalonate pathway for terpenoid biosynthesis. Tetrahedron Letters 1998, 39, 7913-7916
Kuzuyama, T.; Takahashi, S.; Takagi, M.; Seto, H. Characterization of 1-deoxy-D-xylulose 5-phosphate reductoisomerase, an enzyme involved in isopentenyl diphosphate biosynthesis, and identification of its catalytic amino acid residues. J Biol Chem 2000, 275, 19928-32
Leeb, M. Nature 2004, 431, 892-3.
Liu, C. I.; Liu, G. Y.; Song, Y.; Yin, F.; Hensler, M. E.; Jeng, W. Y.; Nizet, V.; Wang, A. H.; Oldfield, E. A cholesterol biosynthesis inhibitor blocks Staphylococcus aureus virulence. Science 2008, 319, 1391-4
Mac Sweeney, A.; Lange, R.; Fernandes, R. P.; Schulz, H.; Dale, G. E.; Douangamath, A.; Proteau, P. J.; Oefner, C. The crystal structure of E. coli 1-deoxy-D-xylulose-5-phosphate reductoisomerase in a ternary complex with the antimalarial compound fosmidomycin and NADPH reveals a tight-binding closed enzyme conformation. J. Mol. Biol. 2005, 345, 115-127
Médecins Sans Frontières Access to Essential Medicines Campaign and the Drugs for Neglected Diseases Working Group, S., Fatal Imbalance: The crisis in research and development for drugs for neglected diseases; www.msf.org.
Merckle, L.; de Andres-Gomez, A.; Dick, B.; Cox, R. J.; Godfrey, C. R. A fragment-based approach to understanding inhibition of 1-deoxy-D-xylulose-5-phosphate reductoisomerase. Chembiochem 2005, 6, 1866-1874.
Meyer, O.; Hoeffler, J.-F.; Grosdemange-Billiard C.; Rohmer, M. Tetrahedron 2004, 60, 12153-62.
Mine, Y.; Kamimura, T.; Nonoyama, S.; Nishida, M.; Goto, S.; Kuwahara, S. In vitro and in vivo antibacterial activities of FR-31564, a new phosphonic acid antibiotic. J. Antibiot. (Tokyo) 1980, 33, 36-43.
Missinou, M. A.; Borrmann, S.; Schindler, A.; Issifou, S.; Adegnika, A. A.; Matsiegui, P. B.; Binder, R.; Lell, B.; Wiesner, J.; Baranek, T.; Jomaa, H.; Kremsner, P. G. Fosmidomycin for malaria. Lancet 2002, 360, 1941-1942.
Molecular Operating Environment (MOE), Montreal, Quebec, 2006
Montoya, J. G.; Liesenfeld, O. Toxoplasmosis. Lancet 2004, 363, 1965-76
Mueller, C.; Schwender, J.; Zeidler, J.; Lichtenthaler, H. K. Properties and inhibition of the first two enzymes of the non-mevalonate pathway of isoprenoid biosynthesis. Biochem Soc Trans 2000, 28, 792-3
Munos, J. W.; Pu, X.; Liu, H. W. Synthesis and analysis of a fluorinated product analogue as an inhibitor for 1-deoxy-D-xylulose 5-phosphate reductoisomerase. Bioorg. Med. Chem. Lett. 2008, 18, 3090-3094.
Murshudov, G. N.; Vagin, A. A.; Dodson, E. J. Refinement of macromolecular structures by the maximum-likelihood method. Acta Crystallogr. D Biol. Crystallogr. 1997, 53, 240-255
Nathan, C. Antibiotics at the crossroads. Nature 2004, 431, 899-902
Neu, H. C.; Kamimura, T. In vitro and in vivo antibacterial activity of FR-31564, a phosphonic acid antimicrobial agent. Antimicrob Agents Chemother 1981, 19, 1013-23
Nguyen-Ba, N.; Lee, N.; Chan, L.; Zacharie, B. Chem. Commun. 2000, 2311-2.
Ortmann, R.; Wiesner, J.; Silber, K.; Klebe, G.; Jomaa, H.; Schlitzer, M. Novel deoxyxylulosephosphate-reductoisomerase inhibitors: fosmidomycin derivatives with spacious acyl residues. Arch. Pharm. (Weinheim) 2007, 340, 483-490.
Oyakhirome, S.; Issifou, S.; Pongratz, P.; Barondi, F.; Ramharter, M.; Kun, J. F.; Missinou, M. A.; Lell, B.; Kremsner, P. G. Randomized controlled trial of fosmidomycin-clindamycin versus sulfadoxine-pyrimethamine in the treatment of Plasmodium falciparum malaria. Antimicrob Agents Chemother 2007, 51, 1869-71
Pace, P.; Di Francesco, M. E.; Gardelli, C.; Harper, S.; Muraglia, E.; Nizi, E.; Orvieto, F.; Petrocchi, A.; Poma, M.; Rowley, M.; Scarpelli, R.; Laufer, R.; González Paz, O.; Monteagudo, E.; Bonelli, F.; Hazuda, D.; Stillmock, K. A.; Summa, V. J Med Chem 2007, 50, 2225-39.
Paterson, D. L. The epidemiological profile of infections with multidrug-resistant Pseudomonas aeruginosa and Acinetobacter species. Clin Infect Dis 2006, 43 Suppl 2, S43-8
Pecoul, B.; Chirac, P.; Trouiller, P.; Pinel, J. Access to essential drugs in poor countries: a lost battle? Jama 1999, 281, 361-7
Perola, E.; Walters, W. P.; Charifson, P. S. A detailed comparison of current docking and scoring methods on systems of pharmaceutical relevance. Proteins 2004, 56, 235-49
Ricagno, S.; Grolle, S.; Bringer-Meyer, S.; Sahm, H.; Lindqvist, Y.; Schneider, G. Crystal structure of 1-deoxy-d-xylulose-5-phosphate reductoisomerase from Zymomonas mobilis at 1.9-A resolution. Biochim Biophys Acta 2004, 1698, 37-44
Rodriguez-Concepcion, M. The MEP pathway: a new target for the development of herbicides, antibiotics and antimalarial drugs. Curr. Pharm. Des. 2004, 10, 2391-2400.
Rohdich, F.; Lauw, S.; Kaiser, J.; Feicht, R.; Kohler, P.; Bacher, A.; Eisenreich, W. Isoprenoid biosynthesis in plants -2C-methyl-D-erythritol-4-phosphate synthase (IspC protein) of Arabidopsis thaliana. Febs J 2006, 273, 4446-58
Sakamoto, Y.; Furukawa, S.; Ogihara, H.; Yamasaki, M. Fosmidomycin resistance in adenylate cyclase deficient (cya) mutants of Escherichia coli. Biosci Biotechnol Biochem 2003, 67, 2030-3
Sanderson, L.; Taylor, G. W.; Aboagye, E. O.; Alao, J. P.; Latigo, J. R.; Coombes, R. C.; Vigushin, D. M. Drug Metab. Dispos. 2004, 32, 1132-8.
Schrödinger Suite 2009, Schrödinger, LLC, New York, N.Y., 2009; Glide, version 5.5, Schrödinger, LLC, New York, N.Y., 2009; Jaguar, version 7.6, Schrödinger, LLC, New York, N.Y., 2009
Schwender, J.; Muller, C.; Zeidler, J.; Lichtenthaler, H. K. Cloning and heterologous expression of a cDNA encoding 1-deoxy-D-xylulose-5-phosphate reductoisomerase of Arabidopsis thaliana. FEBS Lett 1999, 455, 140-4
Shtannikov, A. V.; Sergeeva, E. E.; Biketov, S. F.; Ostrovskii, D. N. Evaluation of in vitro antibacterial activity of fosmidomycin and its derivatives. Antibiot. Khimioter 2007, 52, 3-9.
Silber, K.; Heidler, P.; Kurz, T.; Klebe, G. AFMoC enhances predictivity of 3D QSAR: a case study with DOXP-reductoisomerase. J. Med. Chem. 2005, 48, 3547-3563.
Singh, N.; Cheve, G.; Avery, M. A.; McCurdy, C. R. Targeting the methyl erythritol phosphate (MEP) pathway for novel antimalarial, antibacterial and herbicidal drug discovery: inhibition of 1-deoxy-D-xylulose-5-phosphate reductoisomerase (DXR) enzyme. Curr. Pharm. Des. 2007, 13, 1161-1177.
Song, Y.; Chan, J. M.; Tovian, Z.; Secrest, A.; Nagy, E.; Krysiak, K.; Bergan, K.; Parniak, M. A.; Oldfield, E. Bisphosphonate inhibitors of ATP-mediated HIV-1 reverse transcriptase catalyzed excision of chain-terminating 3′-azido, 3′-deoxythymidine: a QSAR investigation. Bioorg Med Chem 2008, 16, 8959-67
Song, Y.; Lin, F. Y.; Yin, F.; Hensler, M.; Rodrígues-Poveda, C. A.; Mukkamala, D.; Cao, R.; Wang, H.; Morita, C. T.; González-Pacanowska, D.; Nizet, V.; Oldfield, E. Phosphonosulfonates Are Potent, Selective Inhibitors of Dehydrosqualene Synthase and Staphyloxanthin Biosynthesis in Staphylococcus aureus. J Med Chem 2008, 52, 976-88
Song, Y.; Liu, C. I.; Lin, F. Y.; No, J. H.; Hensler, M.; Liu, Y. L.; Jeng, W. Y.; Low, J.; Liu, G. Y.; Nizet, V.; Wang, A. H.; Oldfield, E. Inhibition of Staphyloxanthin Virulence Factor Biosynthesis in Staphylococcus aureus: In Vitro, in Vivo, and Crystallographic Results. J Med Chem 2009, in press
Steinbacher, S.; Kaiser, J.; Eisenreich, W.; Huber, R.; Bacher, A.; Rohdich, F. Structural basis of fosmidomycin action revealed by the complex with 2-C-methyl-D-erythritol 4-phosphate synthase (IspC). Implications for the catalytic mechanism and anti-malaria drug development. J. Biol. Chem. 2003, 278, 18401-18407
Takahashi, S.; Kuzuyama, T.; Watanabe, H.; Seto, H. A 1-deoxy-D-xylulose 5-phosphate reductoisomerase catalyzing the formation of 2-C-methyl-D-erythritol 4-phosphate in an alternative nonmevalonate pathway for terpenoid biosynthesis. Proc Natl Acad Sci USA 1998, 95, 9879-84
Testa, C. A.; Brown, M. J. The methylerythritol phosphate pathway and its significance as a novel drug target. Curr. Pharm. Biotechnol. 2003, 4, 248-259.
Thompson, J. D.; Gibson, T. J.; Plewniak, F.; Jeanmougin, F.; Higgins, D. G. The CLUSTAL_X windows interface: flexible strategies for multiple sequence alignment aided by quality analysis tools. Nucleic Acids Res. 1997, 25, 4876-4882
Trouiller, P.; Olliaro, P.; Torreele, E.; Orbinski, J.; Laing, R.; Ford, N. Drug development for neglected diseases: a deficient market and a public-health policy failure. Lancet 2002, 359, 2188-94
Vagin, A.; Teplyakov, A. MOLREP: an Automated Program for Molecular Replacement. J. Appl. Crystallogr. 1997, 30, 1022-1025
Wada, C. K.; Curr. Top. Med. Chem. 2004, 4, 1255-67
Walsh, C. Where will new antibiotics come from? Nat Rev Microbiol 2003, 1, 65-70
Walsh, T. R. The emergence and implications of metallo-beta-lactamases in Gram-negative bacteria. Clin Microbiol Infect 2005, 11 Suppl 6, 2-9
Walsh, T. R.; Toleman, M. A.; Poirel, L.; Nordmann, P. Metallo-beta-lactamases: the quiet before the storm? Clin Microbiol Rev 2005, 18, 306-25
Woo, Y. H.; Fernandes, R. P.; Proteau, P. J. Evaluation of fosmidomycin analogs as inhibitors of the Synechocystis sp. PCC6803 1-deoxy-D-xylulose 5-phosphate reductoisomerase. Bioorg. Med. Chem. 2006, 14, 2375-2385.
Yajima, S.; Hara, K.; lino, D.; Sasaki, Y.; Kuzuyama, T.; Ohsawa, K.; Seto, H. Structure of 1-deoxy-D-xylulose 5-phosphate reductoisomerase in a quaternary complex with a magnesium ion, NADPH and the antimalarial drug fosmidomycin. Acta Crystallogr. Sect. F Struct. Biol. Cryst. Commun. 2007, 63, 466-470
Yajima, S.; Hara, K.; Sanders, J. M.; Yin, F.; Ohsawa, K.; Wiesner, J.; Jomaa, H.; Oldfield, E. Crystallographic structures of two bisphosphonate:1-deoxyxylulose-5-phosphate reductoisomerase complexes. J. Am. Chem. Soc. 2004, 126, 10824-10825.
Yajima, S.; Nonaka, T.; Kuzuyama, T.; Seto, H.; Ohsawa, K. Crystal structure of 1-deoxy-D-xylulose 5-phosphate reductoisomerase complexed with cofactors: implications of a flexible loop movement upon substrate binding. J. Biochem. 2002, 131, 313-317.
Yamamoto, M.; Kakinuma, M.; Kohmoto, S.; Yamada, K. Bull. Chem. Soc. Japan 1989, 62, 958-60.
Zhang, Y.; Cao, R.; Yin, F.; Hudock, M. P.; Guo, R. T.; Krysiak, K.; Mukherjee, S.; Gao, Y. G.; Robinson, H.; Song, Y.; No, J. H.; Bergan, K.; Leon, A.; Cass, L.; Goddard, A.; Chang, T. K.; Lin, F. Y.; Van Beek, E.; Papapoulos, S.; Wang, A. H.; Kubo, T.; Ochi, M.; Mukkamala, D.; Oldfield, E. Lipophilic bisphosphonates as dual farnesyl/geranylgeranyl diphosphate synthase inhibitors: an X-ray and NMR investigation. J Am Chem Soc 2009, 131, 5153-62

One skilled in the art readily appreciates that the present invention is well adapted to carry out the objectives and obtain the ends and advantages mentioned as well as those inherent therein. Methods, procedures, techniques and kits described herein are presently representative of the preferred embodiments and are intended to be exemplary and are not intended as limitations of the scope. Changes therein and other uses will occur to those skilled in the art which are encompassed within the spirit of the invention or defined by the scope of the pending claims.

What is claimed is:

1. A composition having the general formula 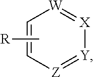
wherein W is independently selected from N or CRi;
X is independently selected from N or CR₂;
Y is independently selected from N or CR₃;
Z is independently selected from N or CR₄;
wherein
R is independently selected from —H, —Cl, —F, —Br, —NO₂, carboxylate, acyl, —CN, sulfone, sulfamide, 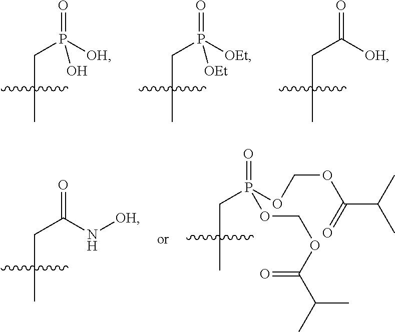 [SMILES: *CP(=O)(O)O ; *CP(=O)(OCC)OCC ; *CC(=O)O ; *CC(=O)NO ; *CP(=O)(OCOC(=O)C(C)C)OCOC(=O)C(C)C]
wherein when W is C, R₁ is independently selected from H, OH, alkyl, alkylaryl, aryl, acyl, 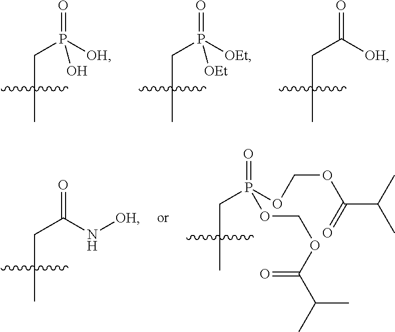 [SMILES: *CP(=O)(O)O ; *CP(=O)(OCC)OCC ; *CC(=O)O ; *CC(=O)NO ; *CP(=O)(OCOC(=O)C(C)C)OCOC(=O)C(C)C]
wherein when X is C, R₂ is independently selected from H, OH, alkyl, aryl, alkylaryl, acyl, 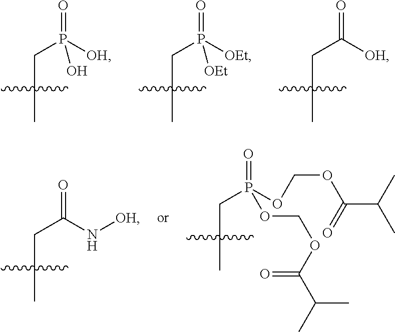 [SMILES: *CP(=O)(O)O ; *CP(=O)(OCC)OCC ; *CC(=O)O ; *CC(=O)NO ; *CP(=O)(OCOC(=O)C(C)C)OCOC(=O)C(C)C]
wherein when Y is C, R₃ is independently selected from H, OH, alkyl, aryl, alkylaryl, acyl 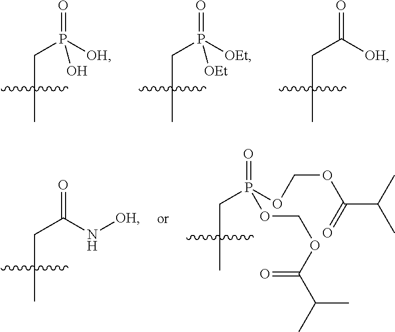 [SMILES: *CP(=O)(O)O ; *CP(=O)(OCC)OCC ; *CC(=O)O ; *CC(=O)NO ; *CP(=O)(OCOC(=O)C(C)C)OCOC(=O)C(C)C]
and
wherein when Z is C, R₄ is independently selected from H, OH, alkyl, aryl, alkylaryl, acyl,

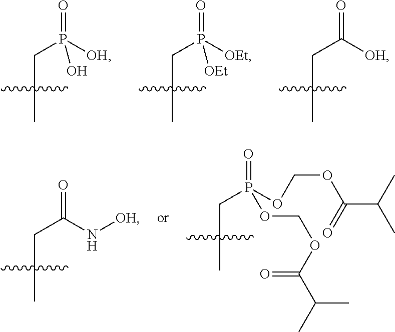 [SMILES: *CP(=O)(O)O ; *CP(=O)(OCC)OCC ; *CC(=O)O ; *CC(=O)NO ; *CP(=O)(OCOC(=O)C(C)C)OCOC(=O)C(C)C]

2. The composition of claim 1, wherein W is CR₁, X is CR₂, Y is CR₃, and Z is N, and
R₁ is independently selected from H, OH, alkyl, alkylaryl, aryl, acyl, 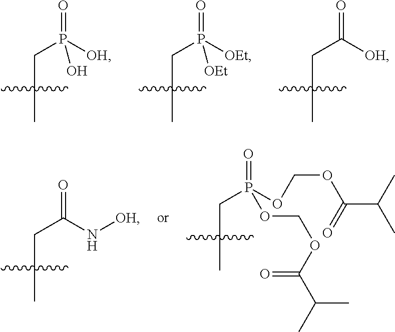 [SMILES: *CP(=O)(O)O ; *CP(=O)(OCC)OCC ; *CC(=O)O ; *CC(=O)NO ; *CP(=O)(OCOC(=O)C(C)C)OCOC(=O)C(C)C]
R₂ is independently selected from H, OH, alkyl, aryl, alkylaryl, or acyl;
R₃ is independently selected from H, OH, alkyl, aryl, alkylaryl, or acyl.

3. The composition of claim 1, wherein W is CR₁, X is CR₂, Y is N, and Z is CR₄, and
R₁ is independently selected from H, OH, alkyl, alkylaryl, aryl, acyl, 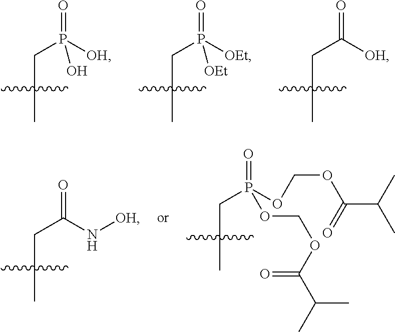 [SMILES: *CP(=O)(O)O ; *CP(=O)(OCC)OCC ; *CC(=O)O ; *CC(=O)NO ; *CP(=O)(OCOC(=O)C(C)C)OCOC(=O)C(C)C]
R₂ is independently selected from H, OH, alkyl, aryl, alkylaryl, or acyl;
R₄ is independently selected from H, OH, alkyl, aryl, alkylaryl, or acyl.

4. The composition of claim 1, wherein, W is CR₁, X is N, Y is CR₃, and Z is CR₄ and
R₁ is independently selected from H, OH, alkyl, alkylaryl, aryl, acyl, 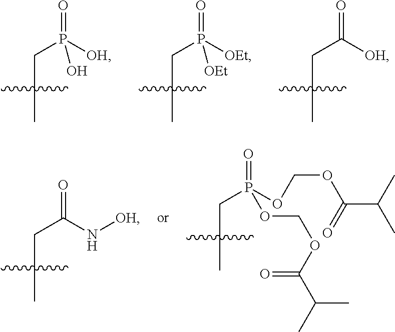 [SMILES: *CP(=O)(O)O ; *CP(=O)(OCC)OCC ; *CC(=O)O ; *CC(=O)NO ; *CP(=O)(OCOC(=O)C(C)C)OCOC(=O)C(C)C]
R₃ is independently selected from H, OH, alkyl, aryl, alkylaryl, or acyl;
R₄ is independently selected from H, OH, alkyl, aryl, alkylaryl, or acyl.

5. The composition of claim 1, wherein W, X, Y and Z form a heteroarenediyl selected from the group consisting of pyridine, quinoline, pyridinium, quinolinium, pyrimidine, purine, and pyrazine.

6. The composition of claim 1 having the structure:

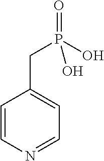 [SMILES: O=P(O)(O)Cc1ccncc1]

7. The composition of claim 1 having the structure:

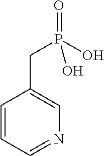 [SMILES: O=P(O)(O)Cc1cccnc1]

8. The composition of claim 1 having the structure:

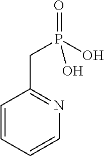 [SMILES: O=P(O)(O)Cc1ccccn1]

9. The composition of claim 1 having the structure:

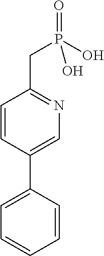 [SMILES: O=P(O)(O)Cc1ccc(-c2ccccc2)cn1]

10. The composition of claim 1 having the structure:

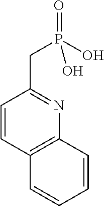 [SMILES: O=P(O)(O)Cc1ccc2ccccc2n1]

11. The composition of claim 1 having one the structure selected from the group consisting of:

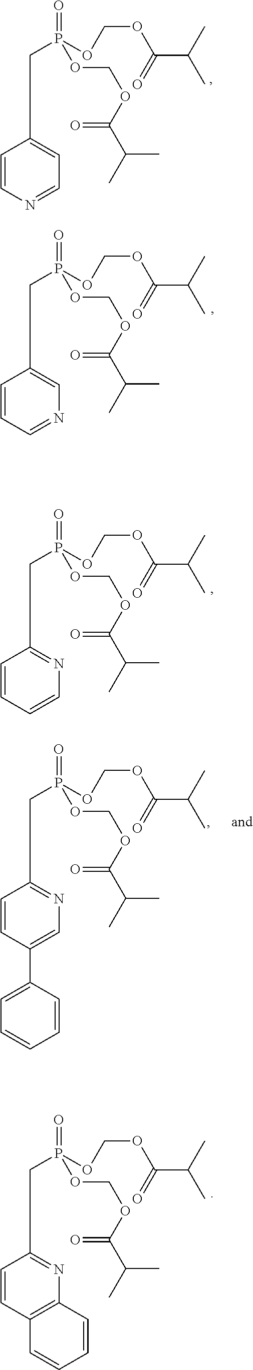

12. A method of treating and/or preventing a microbial infection in an individual, comprising the step of administering a therapeutically effective amount of a composition of claim 1 to the individual.

13. A kit comprising the composition of claim 1.
